SEQUENCE LISTING (35 sequences)

SEQ ID NO: 1 - DNA, 4754 bp, Homo sapiens
gaattctagaggcggcggagggtggcgaggagctctcgctttctctcgctccctccctctccgactccgtctctctctctctctctctctctcccctccctctctttccctctgttccattttttccccctctaaatcctccctgccctgcgcgcctggacacagatttaggaagcgaattcgctcacgttttaggacaaggaagagagagaggcacgggagaagagcccagcaagatttggattgaaaccgagacaccctccggaggctcggagcagaggaaggaggaggagggcggcgaacggaagccagtttgcaattcaagttttgatagcgctggtagaagggggtttaaatcagatttttttttttttaaaggagagagactttttccgctctctcgctccctgttaaagccgggtctagcacagctgcagacgccaccagcgagaaagagggagaggaagacagatagggggcgggggaagaagaaaaagaaaggtaaaaagtcttctaggagaacctttcacatttgcaacaaaagacctaggggctggagagagattcctgggacgcagggctggagtgtctatttcgagctcagcggcagggctcgggcgcgagtcgagaccctgctcgctcctctcgcttctgaaaccgacgttcaggagcggctttttaaaaacgcaaggcacaaggacggtcacccgcgcgactatgtttgctgatttttcgccttgccctctttaaaagcggcctcccattctccaaaagacacttcccctcctccctttgaagtgcattagttgtgatttctgcctccttttcttttttctttcttttttgttttgctttttccccccttttgaattatgtgctgctgttaaacaacaacaaaaaaacaacaaaacacagcagctgcggacttgtccccggctggagcccagcgccccgcctggagtggatgagcctctccatgagagatccggtcattcctgggacaagcatggcctaccatccgttcctacctcaccgggcgccggacttcgccatgagcgcggtgctgggtcaccagccgccgttcttccccgcgctgacgctgcctcccaacggcgcggcggcgctctcgctgccgggcgccctggccaagccgatcatggatcaattggtgggggcggccgagaccggcatcccgttctcctccctggggccccaggcgcatctgaggcctttgaagaccatggagcccgaagaagaggtggaggacgaccccaaggtgcacctggaggctaaagaactttgggatcagtttcacaagcggggcaccgagatggtcattaccaagtcgggaaggcgaatgtttcctccatttaaagtgagatgttctgggctggataaaaaagccaaatacattttattgatggacattatagctgctgatgactgtcgttataaatttcacaattctcggtggatggtggctggtaaggccgaccccgaaatgccaaagaggatgtacattcacccggacagccccgctactggggaacagtggatgtccaaagtcgtcactttccacaaactgaaactcaccaacaacatttcagacaaacatggatttactatattgaactccatgcacaaataccagccccggttccacattgtaagagccaatgacatcttgaaactcccttatagtacatttcggacatacttgttccccgaaactgaattcatcgctgtgactgcataccagaatgataagataacccagttaaaaatagacaacaacccttttgcaaaaggtttccgggacactggaaatggccgaagagaaaaaagaaaacagctcaccctgcagtccatgagggtgtttgatgaaagacacaaaaaggagaatgggacctctgatgagtcctccagtgaacaagcagctttcaactgcttcgcccaggcttcttctccagccgcctccactgtagggacatcgaacctcaaagatttatgtcccagcgagggtgagagcgacgccgaggccgagagcaaagaggagcatggccccgaggcctgcgacgcggccaagatctccaccaccacgtcggaggagccctgccgtgacaagggcagccccgcggtcaaggctcaccttttcgctgctgagcggccccgggacagcgggcggctggacaaagcgtcgcccgactcacgccatagccccgccaccatctcgtccagcactcgcggcctgggcgcggaggagcgcaggagcccggttcgcgagggcacagcgccggccaaggtggaagaggcgcgcgcgctcccgggcaaggaggccttcgcgccgctcacggtgcagacggacgcggccgccgcgcacctggcccagggccccctgcctggcctcggcttcgccccgggcctggcgggccaacagttcttcaacgggcacccgctcttcctgcaccccagccagtttgccatggggggcgccttctccagcatggcggccgctggcatgggtcccctcctggccacggtttctggggcctccaccggtgtctcgggcctggattccacggccatggcctctgccgctgcggcgcagggactgtccggggcgtccgcggccaccctgcccttccacctccagcagcacgtcctggcctctcagggcctggccatgtcccctttcggaagcctgttcccttacccctacacgtacatggccgcagcggcggccgcctcctctgcggcagcctccagctcggtgcaccgccaccccttcctcaatctgaacaccatgcgcccgcggctgcgctacagcccctactccatcccggtgccggtcccggacggcagcagtctgctcaccaccgccctgccctccatggcggcggccgcggggcccctggacggcaaagtcgccgccctggccgccagcccggcctcggtggcagtggactcgggctctgaactcaacagccgctcctccacgctctcctccagctccatgtccttgtcgcccaaactctgcgcggagaaagaggcggccaccagcgaactgcagagcatccagcggttggttagcggcttggaagccaagccggacaggtcccgcagcgcgtccccgtagacccgtcccagacacgtcttttcattccagtccagttcaggctgccgtgcactttgtcggatataaaataaaccacgggcccgccatggcgttagcccttccttttgcagttgcgtctgggaaggggccccggactccctcgagagaatgtgctagagacagcccctgtcttcttggcgtggtttatatgtccgggatctggatcagattctgggggctcagaaacgtcggttgcattgagctactgggggtaggagttccaacatttatgtccagagcaacttccagcaaggctggtctgggtctctgcccaccaggcggggaggtgttcaaagacatctccctcagtgcggatttatatatatatttttccttcactgtgtcaagtggaaacaaaaacaaaatctttcaaaaaaaaaatcgggacaagtgaacacattaacatgattctgtttgtgcagattaaaaactttatagggacttgcattatcggttctcaataaattactgagcagctttgtttggggagggaagtccctaccatccttgtttagtctatattaagaaaatctgtgtctttttaatattcttgtgatgttttcagagccgctgtaggtctcttcttgcatgtccacagtaatgtatttgtggtttttattttgaacgcttgcttttagagagaaaacaatatagccccctacccttttcccaatcctttgccctcaaatcagtgacccaagggagggggggatttaaagggaaggagtgggcaaaacacataaaatgaatttattatatctaagctctgtagcaggattcatgtcgttctttgacagttctttctctttcctgtatatgcaataacaaggttttaaaaaaataataaagaagtgagactattagacaaagtatttatgtaattatttgataactcttgtaaataggtggaatatgaatgcttggaaaattaaactttaatttattgacattgtacatagctctgtgtaaatagaattgcaactgtcaggttttgtgttcttgttttcctttagttgggtttatttccaggtcacagaattgctgttaacactagaaaacacacttcctgcaccaacaccaataccctttcaaaagagttgtctgcaacatttttgttttcttttttaatgtccaaaagtgggggaaagtgctatttcctattttcaccaaaattggggaaggagtgccactttccagctccacttcaaattccttaaaatataactgagattgctgtggggagggaggagggcagaggctgcggtttgactttttaatttttcttttgttatttgtatttgctagtctctgatttcctcaaaacgaagtggaatttactactgttgtcagtatcggtgttttgaattggtgcctgcctatagagatatattcacagttcaaaagtcaggtgctgagagatggtttaaagacaaattcatgaaggtatattttgtgttatagttgttgatgagttctttggttttctgtatttttccccctctctttaaaacatcactgaaatttcaataaatttttattgaaatgtctaaaaaaaaaaaaaaaaaaaaaaaaaaaaaaa

SEQ ID NO: 2 - protein, 723 aa, Homo sapiens
MSLSMRDPVIPGTSMAYHPFLPHRAPDFAMSAVLGHQPPFFPALTLPPNGAAALSLPGALAKPIMDQLVGAAETGIPFSSLGPQAHLRPLKTMEPEEEVEDDPKVHLEAKELWDQFHKRGTEMVITKSGRRMFPPFKVRCSGLDKKAKYILLMDIIAADDCRYKFHNSRWMVAGKADPEMPKRMYIHPDSPATGEQWMSKVVTFHKLKLTNNISDKHGFTILNSMHKYQPRFHIVRANDILKLPYSTFRTYLFPETEFIAVTAYQNDKITQLKIDNNPFAKGFRDTGNGRREKRKQLTLQSMRVFDERHKKENGTSDESSSEQAAFNCFAQASSPAASTVGTSNLKDLCPSEGESDAEAESKEEHGPEACDAAKISTTTSEEPCRDKGSPAVKAHLFAAERPRDSGRLDKASPDSRHSPATISSSTRGLGAEERRSPVREGTAPAKVEEARALPGKEAFAPLTVQTDAAAAHLAQGPLPGLGFAPGLAGQQFFNGHPLFLHPSQFAMGGAFSSMAAAGMGPLLATVSGASTGVSGLDSTAMASAAAAQGLSGASAATLPFHLQQHVLASQGLAMSPFGSLFPYPYTYMAAAAAASSAAASSSVHRHPFLNLNTMRPRLRYSPYSIPVPVPDGSSLLTTALPSMAAAAGPLDGKVAALAASPASVAVDSGSELNSRSSTLSSSSMSLSPKLCAEKEAATSELQSIQRLVSGLEAKPDRSRSASP

SEQ ID NO: 3 - DNA, 4814 bp, Homo sapiens
gaattctagaggcggcggagggtggcgaggagctctcgctttctctcgctccctccctctccgactccgtctctctctctctctctctctctcccctccctctctttccctctgttccattttttccccctctaaatcctccctgccctgcgcgcctggacacagatttaggaagcgaattcgctcacgttttaggacaaggaagagagagaggcacgggagaagagcccagcaagatttggattgaaaccgagacaccctccggaggctcggagcagaggaaggaggaggagggcggcgaacggaagccagtttgcaattcaagttttgatagcgctggtagaagggggtttaaatcagatttttttttttttaaaggagagagactttttccgctctctcgctccctgttaaagccgggtctagcacagctgcagacgccaccagcgagaaagagggagaggaagacagatagggggcgggggaagaagaaaaagaaaggtaaaaagtcttctaggagaacctttcacatttgcaacaaaagacctaggggctggagagagattcctgggacgcagggctggagtgtctatttcgagctcagcggcagggctcgggcgcgagtcgagaccctgctcgctcctctcgcttctgaaaccgacgttcaggagcggctttttaaaaacgcaaggcacaaggacggtcacccgcgcgactatgtttgctgatttttcgccttgccctctttaaaagcggcctcccattctccaaaagacacttcccctcctccctttgaagtgcattagttgtgatttctgcctccttttcttttttctttcttttttgttttgctttttccccccttttgaattatgtgctgctgttaaacaacaacaaaaaaacaacaaaacacagcagctgcggacttgtccccggctggagcccagcgccccgcctggagtggatgagcctctccatgagagatccggtcattcctgggacaagcatggcctaccatccgttcctacctcaccgggcgccggacttcgccatgagcgcggtgctgggtcaccagccgccgttcttccccgcgctgacgctgcctcccaacggcgcggcggcgctctcgctgccgggcgccctggccaagccgatcatggatcaattggtgggggcggccgagaccggcatcccgttctcctccctggggccccaggcgcatctgaggcctttgaagaccatggagcccgaagaagaggtggaggacgaccccaaggtgcacctggaggctaaagaactttgggatcagtttcacaagcggggcaccgagatggtcattaccaagtcgggaaggcgaatgtttcctccatttaaagtgagatgttctgggctggataaaaaagccaaatacattttattgatggacattatagctgctgatgactgtcgttataaatttcacaattctcggtggatggtggctggtaaggccgaccccgaaatgccaaagaggatgtacattcacccggacagccccgctactggggaacagtggatgtccaaagtcgtcactttccacaaactgaaactcaccaacaacatttcagacaaacatggatttactttggccttcccaagtgatcacgctacgtggcaggggaattatagttttggtactcagactatattgaactccatgcacaaataccagccccggttccacattgtaagagccaatgacatcttgaaactcccttatagtacatttcggacatacttgttccccgaaactgaattcatcgctgtgactgcataccagaatgataagataacccagttaaaaatagacaacaacccttttgcaaaaggtttccgggacactggaaatggccgaagagaaaaaagaaaacagctcaccctgcagtccatgagggtgtttgatgaaagacacaaaaaggagaatgggacctctgatgagtcctccagtgaacaagcagctttcaactgcttcgcccaggcttcttctccagccgcctccactgtagggacatcgaacctcaaagatttatgtcccagcgagggtgagagcgacgccgaggccgagagcaaagaggagcatggccccgaggcctgcgacgcggccaagatctccaccaccacgtcggaggagccctgccgtgacaagggcagccccgcggtcaaggctcaccttttcgctgctgagcggccccgggacagcgggcggctggacaaagcgtcgcccgactcacgccatagccccgccaccatctcgtccagcactcgcggcctgggcgcggaggagcgcaggagcccggttcgcgagggcacagcgccggccaaggtggaagaggcgcgcgcgctcccgggcaaggaggccttcgcgccgctcacggtgcagacggacgcggccgccgcgcacctggcccagggccccctgcctggcctcggcttcgccccgggcctggcgggccaacagttcttcaacgggcacccgctcttcctgcaccccagccagtttgccatggggggcgccttctccagcatggcggccgctggcatgggtcccctcctggccacggtttctggggcctccaccggtgtctcgggcctggattccacggccatggcctctgccgctgcggcgcagggactgtccggggcgtccgcggccaccctgcccttccacctccagcagcacgtcctggcctctcagggcctggccatgtcccctttcggaagcctgttcccttacccctacacgtacatggccgcagcggcggccgcctcctctgcggcagcctccagctcggtgcaccgccaccccttcctcaatctgaacaccatgcgcccgcggctgcgctacagcccctactccatcccggtgccggtcccggacggcagcagtctgctcaccaccgccctgccctccatggcggcggccgcggggcccctggacggcaaagtcgccgccctggccgccagcccggcctcggtggcagtggactcgggctctgaactcaacagccgctcctccacgctctcctccagctccatgtccttgtcgcccaaactctgcgcggagaaagaggcggccaccagcgaactgcagagcatccagcggttggttagcggcttggaagccaagccggacaggtcccgcagcgcgtccccgtagacccgtcccagacacgtcttttcattccagtccagttcaggctgccgtgcactttgtcggatataaaataaaccacgggcccgccatggcgttagcccttccttttgcagttgcgtctgggaaggggccccggactccctcgagagaatgtgctagagacagcccctgtcttcttggcgtggtttatatgtccgggatctggatcagattctgggggctcagaaacgtcggttgcattgagctactgggggtaggagttccaacatttatgtccagagcaacttccagcaaggctggtctgggtctctgcccaccaggcggggaggtgttcaaagacatctccctcagtgcggatttatatatatatttttccttcactgtgtcaagtggaaacaaaaacaaaatctttcaaaaaaaaaatcgggacaagtgaacacattaacatgattctgtttgtgcagattaaaaactttatagggacttgcattatcggttctcaataaattactgagcagctttgtttggggagggaagtccctaccatccttgtttagtctatattaagaaaatctgtgtctttttaatattcttgtgatgttttcagagccgctgtaggtctcttcttgcatgtccacagtaatgtatttgtggtttttattttgaacgcttgcttttagagagaaaacaatatagccccctacccttttcccaatcctttgccctcaaatcagtgacccaagggagggggggatttaaagggaaggagtgggcaaaacacataaaatgaatttattatatctaagctctgtagcaggattcatgtcgttctttgacagttctttctctttcctgtatatgcaataacaaggttttaaaaaaataataaagaagtgagactattagacaaagtatttatgtaattatttgataactcttgtaaataggtggaatatgaatgcttggaaaattaaactttaatttattgacattgtacatagctctgtgtaaatagaattgcaactgtcaggttttgtgttcttgttttcctttagttgggtttatttccaggtcacagaattgctgttaacactagaaaacacacttcctgcaccaacaccaataccctttcaaaagagttgtctgcaacatttttgttttcttttttaatgtccaaaagtgggggaaagtgctatttcctattttcaccaaaattggggaaggagtgccactttccagctccacttcaaattccttaaaatataactgagattgctgtggggagggaggagggcagaggctgcggtttgactttttaatttttcttttgttatttgtatttgctagtctctgatttcctcaaaacgaagtggaatttactactgttgtcagtatcggtgttttgaattggtgcctgcctatagagatatattcacagttcaaaagtcaggtgctgagagatggtttaaagacaaattcatgaaggtatattttgtgttatagttgttgatgagttctttggttttctgtatttttccccctctctttaaaacatcactgaaatttcaataaatttttattgaaatgtctaaaaaaaaaaaaaaaaaaaaaaaaaaaaaaa

SEQ ID NO: 4 - protein, 742 aa, Homo sapiens
MSLSMRDPVIPGTSMAYHPFLPHRAPDFAMSAVLGHQPPFFPALTLPPNGAAALSLPGALAKPIMDQLVGAAETGIPFSSLGPQAHLRPLKTMEPEEEVEDDPKVHLEAKELWDQFHKRGTEMVITKSGRRMFPPFKVRCSGLDKKAKYILLMDIIAADDCRYKFHNSRWMVAGKADPEMPKRMYIHPDSPATEQWMSKVVTFHKLKLTNNISDKHGFTLAFPSDHATWQGNYSFGTQTILNSMHKYQPRFHIVRANDILKLPYSTFRTYLFPETEFIAVTAYQNDKITQLKIDNNPFAKGFRDTGNGRREKRKQLTLQSMRVFDERHKKENGTSDESSSEQAAFNCFAQASSPAASTVGTSNLKDLCPSEGESDAEAESKEEHGPEACDAAKISTTTSEEPCRDKGSPAVKAHLFAAERPRDSGRLDKASPDSRHSPATISSSTRGLGAEERRSPVREGTAPAKVEEARALPGKEAFAPLTVQTDAAAAHLAQGPLPGLGFAPGLAGQQFFNGHPLFLHPSQFAMGGAFSSMAAAGMGPLLATVSGASTGVSGLDSTAMASAAAAQGLSGASAATLPFHLQQHVLASQGLAMSPFGSLFPYPYTYMAAAAAASSAAASSSVHRHPFLNLNTMRPRLRYSPYSIPVPVPDGSSLLTTALPSMAAAAGPLDGKVAALAASPASVAVDSGSELNSRSSTLSSSSMSLSPKLCAEKEAATSELQSIQRLVSGLEAKPDRSRSASP

SEQ ID NO: 5 - protein, 600 aa, Homo sapiens
MSLSMRDPVIPGTSMAYHPFLPHRAPDFAMSAVLGHQPPFFPALTLPPNGAAALSLPGALAKPIMDQLVGAAETGIPFSSLGPQAHLRPLKTMEPEEEVEDDPKVHLEAKELWDQFHKRGTEMVITKSGRRMFPPFKVRCSGLDKKAKYILLMDIIAADDCRYKFHNSRWMVAGKADPEMPKRMYIHPDSPATGEQWMSKVVTFHKLKLTNNISDKHGFTLAFPSDHATWQGNYSFGTQTILNSMHKYQPRFHIVRANDILKLPYSTFRTYLFPETEFIAVTAYQNDKITQLKIDNNPFAKGFRDTGNGRREKRKQLTLQSMRVFDERHKKENGTSDESSSEQAAFNCFAQASSPAASTVGTSNLKDLCPSEGESDAEAESKEEHGPEACDAAKISTTTSEEPCRDKGSPAVKAHLFAAERPRDSGRLDKASPDSRHSPATISSSTRGLGAEERRSPVREGTAPAKVEEARALPGKEAFAPLTVQTDAASAAASSSVHRHPFLNLNTMRPRLRYSPYSIPVPVPDGSSLLTTALAASPASVAVDSGSELNSRSSTLSSSSMSLSPKLCAEKEAATSELQSIQRLVSGLEAKPDRSRSASP

SEQ ID NO: 6 - DNA, 6969 bp, Homo sapiens
aatattttgtgtgagagcgagcggtgcatttgcatgttgcggagtgattagtgggtttgaaaagggaaccgtggctcggcctcatttcccgctctggttcaggcgcaggaggaagtgttttgctggaggatgatgacagaggtcaggcttcgctaatgggccagtgaggagcggtggaggcgaggccgggcgccggcacacacacattaacacacttgagccatcaccaatcagcataggaatctgagaattgctctcacacaccaacccagcaacatccgtggagaaaactctcaccagcaactcctttaaaacaccgtcatttcaaaccattgtggtcttcaagcaacaacagcagcacaaaaaaccccaaccaaacaaaactcttgacagaagctgtgacaaccagaaaggatgcctcataaagggggaagactttaactaggggcgcgcagatgtgtgaggccttttattgtgagagtggacagacatccgagatttcagagccccatattcgagccccgtggaatcccgcggcccccagccagagccagcatgcagaacagtcacagcggagtgaatcagctcggtggtgtctttgtcaacgggcggccactgccggactccacccggcagaagattgtagagctagctcacagcggggcccggccgtgcgacatttcccgaattctgcaggtgtccaacggatgtgtgagtaaaattctgggcaggtattacgagactggctccatcagacccagggcaatcggtggtagtaaaccgagagtagcgactccagaagttgtaagcaaaatagcccagtataagcgggagtgcccgtccatctttgcttgggaaatccgagacagattactgtccgagggggtctgtaccaacgataacataccaagcgtgtcatcaataaacagagttcttcgcaacctggctagcgaaaagcaacagatgggcgcagacggcatgtatgataaactaaggatgttgaacgggcagaccggaagctggggcacccgccctggttggtatccggggacttcggtgccagggcaacctacgcaagatggctgccagcaacaggaaggagggggagagaataccaactccatcagttccaacggagaagattcagatgaggctcaaatgcgacttcagctgaagcggaagctgcaaagaaatagaacatcctttacccaagagcaaattgaggccctggagaaagagtttgagagaacccattatccagatgtgtttgcccgagaaagactagcagccaaaatagatctacctgaagcaagaatacaggtatggttttctaatcgaagggccaaatggagaagagaagaaaaactgaggaatcagagaagacaggccagcaacacacctagtcatattcctatcagcagtagtttcagcaccagtgtctaccaaccaattccacaacccaccacaccggtttcctccttcacatctggctccatgttgggccgaacagacacagccctcacaaacacctacagcgctctgccgcctatgcccagcttcaccatggcaaataacctgcctatgcaacccccagtccccagccagacctcctcatactcctgcatgctgcccaccagcccttcggtgaatgggcggagttatgatacctacacccccccacatatgcagacacacatgaacagtcagccaatgggcacctcgggcaccacttcaacaggactcatttcccctggtgtgtcagttccagttcaagttcccggaagtgaacctgatatgtctcaatactggccaagattacagtaaaaaaaaaaaaaaaaaaaaaaaggaaaggaaatattgtgttaattcagtcagtgactatggggacacaacagttgagctttcaggaaagaaagaaaaatggctgttagagccgcttcagttctacaattgtgtcctgtattgtaccactggggaaggaatggacttgaaacaaggacctttgtatacagaaggcacgatatcagttggaacaaatcttcattttggtatccaaacttttattcattttggtgtattatttgtaaatgggcatttgtatgttataatgaaaaaaagaacaatgtagactggatggatgtttgatctgtgttggtcatgaagttgttttttttttttttaaaaagaaaaccatgatcaacaagctttgccacgaatttaagagttttatcaagatatatcgaatacttctacccatctgttcatagtttatggactgatgttccaagtttgtatcattcctttgcatataattaaacctggaacaacatgcactagatttatgtcagaaatatctgttggttttccaaaggttgttaacagatgaagtttatgtgcaaaaaagggtaagatataaattcaaggaagaaaaaaagttgatagctaaaaggtagagtgtgtcttcgatataatccaatttgttttatgtcaaaatgtaagtatttgtcttccctagaaatcctcagaatgatttctataataaagttaatttcatttatatttgacaagaatatagatgttttatacacattttcatgcaatcatacgtttcttttttggccagcaaaagttaattgttcttagatatagttgtattactgttcacggtccaatcattttgtgcatctagagttcattcctaatcaattaaaagtgcttgcaagagttttaaacttaagtgttttgaagttgttcacaactacatatcaaaattaaccattgttgattgtaaaaaaccatgccaaagcctttgtatttcctttattatacagttttctttttaaccttatagtgtggtgttacaaattttatttccatgttagatcaacattctaaaccaatggttactttcacacacactctgttttacatcctgatgatccttaaaaaataatccttatagataccataaatcaaaaacgtgttagaaaaaaattccacttacagcagggtgtagatctgtgcccatttatacccacaacatatatacaaaatggtaacatttcccagttagccatttaattctaaagctcaaagtctagaaataatttaaaaatgcaacaagcgattagctaggaattgttttttgaattaggactggcattttcaatctgggcagatttccattgtcagcctatttcaacaatgatttcactgaagtatattcaaaagtagatttcttaaaggagactttctgaaagctgttgcctttttcaaataggccctctcccttttctgtctccctcccctttgcacaagaggcatcatttcccattgaaccactacagctgttcccatttgaatcttgctttctgtgcggttgtggatggttggagggtggaggggggatgttgcatgtcaaggaataatgagcacagacacatcaacagacaacaacaaagcagactgtgactggccggtgggaattaaaggccttcagtcattggcagcttaagccaaacattcccaaatctatgaagcagggcccattgttggtcagttgttatttgcaatgaagcacagttctgatcatgtttaaagtggaggcacgcagggcaggagtgcttgagcccaagcaaaggatggaaaaaaataagcctttgttgggtaaaaaaggactgtctgagactttcatttgttctgtgcaacatataagtcaatacagataagtcttcctctgcaaacttcactaaaaagcctgggggttctggcagtctagattaaaatgcttgcacatgcagaaacctctggggacaaagacacacttccactgaattatactctgctttaaaaaaatccccaaaagcaaatgatcagaaatgtagaaattaatggaaggatttaaacatgaccttctcgttcaatatctactgttttttagttaaggaattacttgtgaacagataattgagattcattgctccggcatgaaatatactaataattttattccaccagagttgctgcacatttggagacaccttcctaagttgcagtttttgtatgtgtgcatgtagttttgttcagtgtcagcctgcactgcacagcagcacatttctgcaggggagtgagcacacatacgcactgttggtacaattgccggtgcagacatttctacctcctgacattttgcagcctacattccctgagggctgtgtgctgagggaactgtcagagaagggctatgtgggagtgcatgccacagctgctggctggcttacttcttccttctcgctggctgtaatttccaccacggtcaggcagccagttccggcccacggttctgttgtgtagacagcagagactttggagacccggatgtcgcacgccaggtgcaagaggtgggaatgggagaaaaggagtgacgtgggagcggagggtctgtatgtgtgcacttgggcacgtatatgtgtgctctgaaggtcaggattgccagggcaaagtagcacagtctggtatagtctgaagaagcggctgctcagctgcagaagccctctggtccggcaggatgggaacggctgccttgccttctgcccacaccctagggacatgagctgtccttccaaacagagctccaggcactctcttggggacagcatggcaggctctgtgtggtagcagtgcctgggagttggccttttactcattgttgaaataatttttgtttattatttatttaacgatacatatatttatatatttatcaatggggtatctgcagggatgttttgacaccatcttccaggatggagattatttgtgaagacttcagtagaatcccaggactaaacgtctaaattttttctccaaacttgactgacttgggaaaaccaggtgaatagaataagagctgaatgttttaagtaataaacgttcaaactgctctaagtaaaaaaatgcattttactgcaatgaatttctagaatatttttcccccaaagctatgcctcctaacccttaaatggtgaacaactggtttcttgctacagctcactgccatttcttcttactatcatcactaggtttcctaagattcactcatacagtattatttgaagattcagctttgttctgtgaatgtcatcttaggattgtgtctatattcttttgcttatttctttttactctgggcctctcatactagtaagattttaaaaagccttttcttctctgtatgtttggctcaccaaggcgaaatatatattcttctctttttcatttctcaagaataaacctcatctgcttttttgtttttctgtgttttggcttggtactgaatgactcaactgctcggttttaaagttcaaagtgtaagtacttagggttagtactgcttatttcaataatgttgacggtgactatctttggaaagcagtaacatgctgtcttagaaatgacattaataatgggcttaaacaaatgaataggggggtccccccactctccttttgtatgcctatgtgtgtctgatttgttaaaagatggacagggaattgattgcagagtgtcgcttccttctaaagtagttttattttgtctactgttagtatttaaagatcctggaggtggacataaggaataaatggaagagaaaagtagatattgtatggtggctactaaaaggaaattcaaaaagtcttagaacccgagcacctgagcaaactgcagtagtcaaaatatttatctcatgttaaagaaaggcaaatctagtgtaagaaatgagtaccatatagggttttgaagttcatatactagaaacacttaaaagatatcatttcagatattacgtttggcattgttcttaagtatttatatctttgagtcaagctgataattaaaaaaaatctgttaatggagtgtatatttcataatgtatcaaaatggtgtctatacctaaggtagcattattgaagagagatatgtttatgtagtaagttattaacataatgagtaacaaataatgtttccagaagaaaggaaaacacattttcagagtgcgtttttatcagaggaagacaaaaatacacacccctctccagtagcttatttttacaaagccggcccagtgaattagaaaaacaaagcacttggatatgatttttggaaagcccaggtacacttattattcaaaatgcacttttactgagtttgaaaagtttcttttatatttaaaataagggttcaaatatgcatattcaatttttatagtagttatctatttgcaaagcatatattaactagtaattggctgttaattttatagacatggtagccagggaagtatatcaatgacctattaagtattttgacaagcaatttacatatctgatgacctcgtatctctttttcagcaagtcaaatgctatgtaattgttccattgtgtgttgtataaaatgaatcaacacggtaagaaaaaggttagagttattaaaataataaactgactaaaatactcatttgaatttattcagaatgttcataatgctttcaaaggacatagcagagcttttgtggagtatccgcacaacattatttattatctatggactaaatcaattttttgaagttgctttaaaatttaaaagcacctttgcttaatataaagccctttaattttaactgacagatcaattctgaaactttattttgaaaagaaaatggggaagaatctgtgtctttagaattaaaagaaatgaaaaaaataaacccgacattctaaaaaaatagaataagaaacctgatttttagtactaatgaaatagcgggtgacaaaatagttgtctttttgattttgatcacaaaaaataaactggtagtgacaggatatgatggagagatttgacatcctggcaaatcactgtcattgattcaattattctaattctgaataaaagctgtatacagtaaaa

SEQ ID NO: 7 - protein, 422 aa, Homo sapiens [sequence omitted]

SEQ ID NO: 8 - DNA, 25 bp, Artificial Sequence
  primer
gatcggatccagaagttgctgcttg

SEQ ID NO: 9 - DNA, 27 bp, Artificial Sequence
  primer
gatcccatggtcactttatctcacagc

SEQ ID NO: 10 - DNA, 25 bp, Artificial Sequence
  primer
gatcggatccagatgttgctgcttg

SEQ ID NO: 11 - DNA, 26 bp, Artificial Sequence
  primer
gatcccatggtcacttgcaccctttg

SEQ ID NO: 12 - DNA, 24 bp, Artificial Sequence
  primer
ggccatatgccctctgaaaatctg

SEQ ID NO: 13 - DNA, 25 bp, Artificial Sequence
  primer
agttctagaggctcgaggttttttg

SEQ ID NO: 14 - DNA, 22 bp, Artificial Sequence
  primer
gctcgaattcaaggccgagctg

SEQ ID NO: 15 - DNA, 26 bp, Artificial Sequence
  primer
gatcctcgagctactctcctcgtcac

SEQ ID NO: 16 - DNA, 23 bp, Artificial Sequence
  primer
gtttaaagaattcatggccctgg

SEQ ID NO: 17 - DNA, 25 bp, Artificial Sequence
  primer
agttctagaggctcgaggttttttg

SEQ ID NO: 18 - DNA, 21 bp, Artificial Sequence
  primer
ggcagacactatcagcctgcc

SEQ ID NO: 19 - DNA, 26 bp, Artificial Sequence
  primer
gcacaggcctatgataaagttatccc

SEQ ID NO: 20 - DNA, 25 bp, Artificial Sequence
  primer
tacagaacccggactgtcccagtca

SEQ ID NO: 21 - DNA, 25 bp, Artificial Sequence
  primer
ctgttcccagagatccttggcttcc

SEQ ID NO: 22 - DNA, 24 bp, Artificial Sequence
  primer
cgggataacattcagggtatcact

SEQ ID NO: 23 - DNA, 24 bp, Artificial Sequence
  primer
atccatggcggtaactgtcttcct

SEQ ID NO: 24 - DNA, 18 bp, Artificial Sequence
  primer
cacagttccaccaaatgc

SEQ ID NO: 25 - DNA, 18 bp, Artificial Sequence
  primer
ggaatcaagcggtacaga

SEQ ID NO: 26 - DNA, 20 bp, Artificial Sequence
  primer
acacggcattgatcctacag

SEQ ID NO: 27 - DNA, 20 bp, Artificial Sequence
  primer
agctccttcggtgtaatgac

SEQ ID NO: 28 - DNA, 18 bp, Artificial Sequence
  primer
ggatcgttatcacctctg

SEQ ID NO: 29 - DNA, 18 bp, Artificial Sequence
  primer
gtgtagtctgtagcagca

SEQ ID NO: 30 - DNA, 18 bp, Artificial Sequence
  primer
gctgacagaatgcagaag

SEQ ID NO: 31 - DNA, 18 bp, Artificial Sequence
  primer
ttgcttggaggagtgtgt

SEQ ID NO: 32 - DNA, 21 bp, Artificial Sequence
  primer
ccagtaatttcagggtcaggc

SEQ ID NO: 33 - DNA, 20 bp, Artificial Sequence
  primer
aagaacactcacaaatcatg

SEQ ID NO: 34 - DNA, 23 bp, Artificial Sequence
  primer
gctgttctgtattaaagtcctgg

SEQ ID NO: 35 - DNA, 23 bp, Artificial Sequence
  primer
ggaaaggagataacacgagttgg

(19) United States
(12) Patent Application Publication
(10) Pub. No.: US 2017/0321188 A1
(43) Pub. Date: Nov. 9, 2017

(54) METHODS OF GENERATING RETINAL PROGENITOR CELL PREPARATIONS AND USES THEREOF

(71) Applicant: The Research Foundation for The State University of New York, Syracuse, NY (US)

(72) Inventors: Andrea S. VICZIAN, Manlius, NY (US); Michael E. ZUBER, Manlius, NY (US)

(21) Appl. No.: 15/587,254

(22) Filed: May 4, 2017

Related U.S. Application Data
(60) Provisional application No. 62/331,861, filed on May 4, 2016.

(51) Int. Cl. C12N 5/0797; A61K 35/30

(52) U.S. Cl. CPC C12N 5/0623; A61K 35/30; C12N 2533/90; C12N 2506/02

(57) ABSTRACT

The present invention relates to methods of generating preparations of neural progenitor cells and retinal progenitor cells from populations of stem cells. These methods involve the administration of Tbx3 alone or in combination with Pax6. The preparations of neural and retinal progenitor cells prepared in accordance with the methods disclosed herein are suitable for use in methods of treating individuals having retinal disorders.

This application claims the priority benefit of U.S. Provisional Patent Application Ser. No. 62/331,861, filed on May 4, 2016, which is hereby incorporated by reference in its entirety.

### FIELD OF THE INVENTION

The present invention relates to methods and kits for generating retinal progenitor cells that involve the two required transcription factors of Tbx3 and Pax6.

### BACKGROUND OF THE INVENTION

Normal brain development requires the coordinated activity of both extrinsic and intrinsic regulators. These factors first repress bone morphogenic proteins (BMP) signaling in the early ectoderm to induce the formation of multipotent neural progenitor cells, then specify and determine the neural plate to form distinct regions of the adult nervous system. High levels of BMP signaling specify epidermis, while low BMP signaling results in a neural fate. Excessive bmp4 expression in the anterior neural plate results in a reduction or total absence of anterior neural structures, including eyes (Hartley et al., “Transgenic Xenopus Embryos Reveal that Anterior Neural Development Requires Continued Suppression of BMP Signaling After Gastrulation,” Dev Biol 238:168-184 (2001), and Hartley et al., “Targeted Gene Expression In Transgenic Xenopus Using The Binary Gal4-Uas System,” Proc Natl Acad Sci USA 99:1377-1382 (2002)). Noggin, and other BMP antagonists, bind BMP and prevent it from activating BMP receptors (Lamb et al., “Neural Induction By The Secreted Polypeptide Noggin,” Science 262: 713-718 (1993), and Re'em-Kalma et al., “Competition Between Noggin And Bone Morphogenetic Protein 4 Activities May Regulate Dorsalization During Xenopus Development,” Proc Natl Acad Sci USA 92:12141-12145 (1995)). It has been assumed that Noggin also indirectly regulates bmp4 transcription, since BMP4 protein can regulate its own transcription in a positive autoregulatory feedback loop (Hammerschmidt et al., “Genetic Analysis Of Dorsoventral Pattern Formation In The Zebrafish: Requirement Of A Bmp-Like Ventralizing Activity And Its Dorsal Repressor,” Genes Dev 10:2452-2461 (1996), Jones et al., “Dvr-4 (Bone Morphogenetic Protein-4) As A Posterior-Ventralizing Factor In Xenopus Mesoderm Induction,” Development 115: 639-647 (1992), Piccolo et al., “Cleavage Of Chordin By Xolloid Metalloprotease Suggests A Role For Proteolytic Processing In The Regulation Of Spemann Organizer Activity,” Cell 91:407-416 (1997), Gestri et al., “Six3 Functions In Anterior Neural Plate Specification By Promoting Cell Proliferation And Inhibiting Bmp4 Expression,” Development 132:2401-2413 (2005), Gammill et al., “Coincidence Of Otx2 And Bmp4 Signaling Correlates With Xenopus Cement Gland Formation,” Mech Dev 92:217-226 (2000), and Schmidt et al., “Localized Bmp-4 Mediates Dorsal/Ventral Patterning In The Early Xenopus Embryo,” Dev Biol 169:37-50 (1995)). Together, these activities result in pluripotent ectoderm cells being determined to form multipotent neural, then retinal progenitors. Noggin not only specifies pluripotent cells to retina in the context of the eye field, but also determines cells to form retina on the embryonic flank and even in culture (Viczian et al., “Tissue Determination Using the Animal Xap Transplant (ACT) Assay in Xenopus laevis,” J Vis Exp 39:1932 (2010), Wong et al., “Efficient Retina Formation Requires Suppression Of Both Activin And Bmp Signaling Pathways In Pluripotent Cells,” Biol Open 4:573-583 (2015), and Lan et al., “Noggin Elicits Retinal Fate In Xenopus Animal Cap Embryonic Stem Cells,” Stem Cells 27:2146-2152 (2009)).

In Xenopus laevis, the eye field transcription factor (EFTF) Tbx3 was originally identified as ET (eye T-box) (Li et al., “A Single Morphogenetic Field Gives Rise To Two Retina Primordia Under The Influence Of The Prechordal Plate,” Development 124:603-615 (1979)). In comparison to other eye field transcription factors, Tbx3 has the most restricted eye field expression domain and is expressed prior to all EFTFs but Six3 (Zuber et al., “Specification Of The Vertebrate Eye By A Network Of Eye Field Transcription Factors,” Development 130:5155-5167 (2003)). Tbx3 functions downstream of Noggin and upstream of other EFTFs, and is a necessary component of the eye field transcription factor network sufficient to induce ectopic and functional eyes (Zuber et al., “Specification Of The Vertebrate Eye By A Network Of Eye Field Transcription Factors,” Development 130:5155-5167 (2003), and Viczian et al., “Generation of functional eyes from pluripotent cells,” PLoS Biol 7:e1000174 (2009)). In direct contrast to other EFTFs, Tbx3 misexpression has not been reported to induce ectopic retina or even enlarge the retina in Xenopus embryos (Mathers et al., “The Rx Homeobox Gene Is Essential For Vertebrate Eye Development,” Nature 387:603-607 (1997), Bernier et al., “Expanded Retina Territory By Midbrain Transformation Upon Overexpression Of Six6 (Optx2) In Xenopus Embryos”, Mech Dev 93:59-69 (2000), Andreazzoli et al., “Role Of Xrx1 In Xenopus Eye And Anterior Brain Development,” Development 126:2451-2460 (1999), Chow et al., “Pax6 Induces Ectopic Eyes In A Vertebrate,” Development 126: 4213-4222 (1999), and Zuber et al., “Giant Eyes In Xenopus Laevis By Overexpression Of Xoptx2,” Cell 98:341-352 (1999)), suggesting Tbx3 plays a minor role if any in retinal development. Thus, there has been little interest in further investigating Tbx3 in eye formation.

Although expressed in the developing mouse eye, no eye phenotype has been reported in Tbx3 null mice, which die during early embryogenesis (Davenport et al., “Mammary Gland, Limb And Yolk Sac Defects In Mice Lacking Tbx3, The Gene Mutated In Human Ulnar Mammary Syndrome,” Development 130:2263-2273 (2003), and Ribeiro et al., “Tbx2 And Tbx3 Regulate The Dynamics Of Cell Proliferation During Heart Remodeling,” PLoS One 2:e398 (2007)). Tbx3 is important for both the establishment and maintenance of stem cell pluripotency and can inhibit differentiation of progenitor cells, yet its role in early eye formation has not been determined (Davenport et al., “Mammary Gland, Limb And Yolk Sac Defects In Mice Lacking Tbx3, The Gene Mutated In Human Ulnar Mammary Syndrome,” Development 130:2263-2273 (2003), Lu et al., “Dual Functions Of T-Box 3 (Tbx3) In The Control Of Self-Renewal And Extraembryonic Endoderm Differentiation In Mouse Embryonic Stem Cells,” J Biol Chem 286:8425-8436 (2011), and Ivanova et al., “Dissecting Self-Renewal In Stem Cells With RNA Interference,” Nature 442:533-538 (2006)).

The present invention is directed to overcoming these and other deficiencies in the art.

### SUMMARY OF THE INVENTION

One aspect of the present invention relates to a method of producing an enriched preparation of neural progenitor cells from a population of pluripotent stem cells. The method comprises administering Tbx3 to the population of pluripotent stem cells and culturing the population of pluripotent stem cells, to which Tbx3 has been administered, under conditions suitable to produce the enriched preparation of neural progenitor cells from the population of pluripotent stem cells.

Another aspect of the present invention relates to an enriched preparation of neural progenitor cells produced in accordance with the methods of the present invention.

Another aspect of the present invention relates to a method of treating a retinal disorder. The method comprises selecting a subject having a retinal disorder, and administering, to the subject, the enriched preparation of neural progenitor cells produced in accordance with the methods of the present invention.

Another aspect of the present invention relates to a method of treating a spinal cord injury or traumatic brain injury in a subject. The method comprises selecting a subject having a spinal cord injury or traumatic brain injury, and administering, to said subject, the enriched population of neural progenitor cells produced in accordance with the methods of the present invention.

Another aspect of the present invention relates to a method of producing an enriched preparation of retinal progenitor cells from a population of stem cells. The method comprises administering Tbx3 and Pax6 to the population of stem cells and culturing the population of stem cells, to which Tbx3 and Pax6 have been administered, under conditions suitable to produce the enriched preparation of retinal progenitor cells from the population of stem cells.

Another aspect of the present invention relates to a preparation of retinal organoids formed in accordance with the methods of the present invention.

Another aspect of the present invention relates to an enriched preparation of retinal progenitor cells produced in accordance with the methods of the present invention.

Another aspect of the present invention relates to a method of treating a retinal disorder. The method comprises selecting a subject having a retinal disorder, and administering, to the subject, the enriched preparation of retinal progenitor cells produced in accordance with the methods of the present invention.

Vertebrate eye formation begins in the anterior neural plate in a region called the eye field, which is first specified, then determined to form the retina. Eye field transcription factors or EFTFs, are expressed in eye field cells, are necessary, and in combination sufficient for retinal determination. Tbx3 can regulate the expression of most EFTFs; however its role in retinal specification and determination is unknown. As described herein, Tbx3 is required for normal eye formation. Although sufficient for neural determination, Tbx3 is only sufficient to specify a retinal lineage in the context of the eye field. Unlike Tbx3, Noggin, which induces pax6, is sufficient to determine a retinal lineage in pluripotent cells. In combination, Tbx3 and Pax6 are sufficient to reprogram pluripotent cells to a retinal lineage. The data described herein indicate that Tbx3 inhibits bmp4 expression, and maintains eye field neural progenitors in a multipotent state, and in combination with Pax6, Tbx3 determines eye field cells to form retina.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1F demonstrate that Tbx3 is sufficient to specify pluripotent cells to a retinal lineage. FIG. 1A shows a schematic illustrating the Animal Cap Transplant (ACT) Assay to Eye Field (ACT→EF) (Viczian and Zuber, J. Vis. Exp. 39:1932 (2010), which is hereby incorporated by reference in its entirety). FIG. 1B depicts a histogram showing the percent of stage 43 tadpoles in which transplanted cells formed retina in response to the expression of YFP only, or YFP with the indicated EFTF, an EFTF cocktail or Noggin (YFP-only, 500 pg, n=40; Otx2, 25 pg, n=41; Tbx3, 50 pg, n=43; Rax, 50 pg n=40; Pax6, 100 pg, n=42; Six3, 25 pg, n=41; Six6, 25 pg, n=40; Nr2e1, 25 pg, n=41; EFTF cocktail, n=40; Noggin, 2.5 pg, n=40). FIGS. 1C-F show transverse section of stage 43 retinas from embryos receiving transplants expressing YFP (FIG. 1C), YFP and Noggin (FIG. 1D), YFP and Pax6 (FIG. 1E), or YFP and Tbx3 (FIG. 1F). Sections were stained for XAP-2 (red), DAPI (blue), and vYFP (green) to detect rod outer segments, nuclei, and transplanted donor cells, respectively. Dorsal retina is the top of each panel. Error bars are standard error of the mean; * P≦0.05 by one-way ANOVA; scale bar, 50 μm.

FIGS. 2A-2I demonstrate that tbx3 is expressed in a pattern consistent with a role in eye formation. FIGS. 2A-D show posterior (FIGS. 2A and 2E) and anterior (FIGS. 2B-2D) views of intact embryos showing the expression pattern of tbx3 at the indicated developmental stages. FIGS. 2F-2H show stage 15 embryos stained by whole mount in situ hybridization, then cut midsagittal (FIGS. 2F,G) and parasagittal (FIG. 2H) to reveal internal tissues expressing tbx3. FIG. 2I shows RT-PCR of isolated eye fields at the indicated stages detecting the expression of transcripts for tbx3.L and tbx3.S homologs. Abbreviations: dbl, dorsal blastopore lip; anp, anterior neural plate; ef, eye field; cg, cement gland; ef-m, eye field—midline; ef-a, eye field—eye anlagen; bp, blastopore; vim, ventral involuting mesoderm; dm, dorsal mesoderm; arch, archenteron; pp, prechordal plate; sne, sensorial layer of neuroectoderm; ene, epithelial layer of neuroectoderm; vm, ventral mesoderm; 18-RT, stage 18 minus RT control. Scale bar, 200 μm.

FIGS. 3A-3H demonstrate that tbx3 is required for normal eye formation. Design and test of Tbx3 morpholino activity are as follows. FIG. 3A shows a sequence alignment of X. laevis tbx3.L and tbx3.S homeologs and the relative position of the Tbx3MO-LS and Tbx3MO-S morpholino target sequences in light and dark blue, respectively. FIG. 3B shows western blot detection of the expression of YFP and β-actin (loading control) in extracts prepared from embryos injected in both blastomeres at the two-cell stage with 10 ng of the indicated morpholino, and cRNA coding for Tbx3.L/.S-YFP fusion proteins. In FIGS. 3C-3F, eye defects following Tbx3 knockdown are shown. Injected side of tadpoles treated with 10 ng CoMO (FIG. 3C), 10 ng Tbx3MO-S (FIG. 3D) or 10 ng Tbx3MO-LS (FIG. 3E) are shown. FIG. 3F shows the uninjected side of the same embryo shown in FIG. 3E. The percent reduction in eye size after morpholino injection was determined by comparing the dorsoventral (D/V) eye diameter on the uninjected and injected sides. Histograms show reduction in eye size of embryos injected with vYFP RNA and the indicated morpholino (FIG. 3G) or combination of morpholinos (FIG. 3H). Error bars show the s.e.m. P-values calculated using a one-way ANOVA analysis (ns P>0.05; ****P≦0.0001). Scale bars, 200 μm.

FIGS. 4A-4J demonstrate that Tbx3 is required for normal eye formation. FIGS. 4A-4B depict constructs used to test morpholino activity in the whole embryo. FIGS. 4C-H″ show bright-field (FIGs. C-H), mCherry fluorescence (FIGs. C′-H′) and vYFP fluorescence (FIGs. C″-H″) images of neurula stage embryos. Neurula stage embryos were unilaterally injected at the two-cell stage (right side-reader's perspective) with cRNA for mCherry and Tbx3-L-vYFP (FIGs. A, C-E″) or Tbx3-S-vYFP (FIGs. B, F-H″) as diagrammed above the panels. CoMO (C-C″, n=54; F-F″, n=57), Tbx3MO-LS (D-D″, n=60; G-G″, n=59) and Tbx3MO-S (E-E″, n=58; H-H″, n=60) were also injected to determine if translation of the vYFP fusion constructs was blocked by each morpholino. Scale bar, 200 μm. FIGS. 4I-4J show the percent reduction in eye size determined by comparing the anteroposterior diameter of the eye on the uninjected side to the injected side. Histograms show eye size differential measured in wild-type animals and tadpoles injected as embryos with vYFP and the indicated morpholino or combination of morpholinos. Error bars show the s.e.m. P-values calculated using a one-way ANOVA analysis (ns, P>0.05; ****, P≦0.0001).

FIGS. 5A-5H demonstrate that splice blocking phenocopies eye defects observed with translation blocking Tbx3 morpholinos. The splice blocking morpholino (Tbx3MO-SP) was designed to the exon 1 splice donor site of tbx3.L and tbx3.S. An in frame stop codon is located in intron 1 immediately following the splice-donor site, resulting in truncation of the protein. FIG. 5A shows a schematic of Tbx3 gene structure, location of the splice blocking morpholino, and PCR primers used to confirm altered splicing. FIG. 5B shows an alignment of tbx3.L and tbx3.S target sites with location of Tbx3MO-SP. Uppercase and lowercase nucleotides identify exon and intron regions, respectively. In frame intronic stop codon (tga) is underlined. FIG. 5C shows the results of RT-PCR to detection of unspliced tbx3.S (FR1) and tbx3.L (FR2) transcripts. An increase in unspliced tbx3.S and tbx3.L transcripts is detected in Tbx3MO-SP (MO-SP) injected embryos relative to YFP and control (CoMO) morpholino injected embryos. FIGS. 5D-5F show eye defects following splice-blocking of Tbx3 transcript. Injected side of tadpoles treated with Tbx3MO-SP (FIG. 5F, n=87) is shown with the CoMO (FIG. 5D) and Tbx3MO-LS (FIG. 5E) injected tadpoles from FIGS. 3C and 3E for comparison purposes. FIGS. 5G-5H show the percent reduction in eye size determined by comparing the dorsoventral and anteroposterior diameters of the eye on the injected side relative to the uninjected side. Histograms show eye size differential measured in tadpoles injected in one blastomere at the two cell stage with the indicated morpholino. Error bars show mean±s.e.m. P-values calculated using a one-way ANOVA analysis (****P≦0.0001); N=2; Scale bars, 200 μm.

FIGS. 6A-6J′ demonstrate that Tbx3 knockdown inhibits the retinal and neural inducing activity of Noggin. FIGS. 6A-F show transverse sections of stage 43 retinas from embryos receiving cell transplants at stage 15 (ACT→EF). FIGS. 6A-C show donor cells expressed YFP-only (FIG. 6A), or were coinjected with CoMO (FIG. 6B), or Tbx3MO-LS (Tbx3MO) (FIG. 6C). FIGS. 6D-6F show donor cells expressed YFP plus Noggin (Nog) alone (FIG. 6D), or in combination with CoMO (FIG. 6E), or Tbx3MO (FIG. 6F). FIG. 6G shows a histogram showing the average percent of tadpoles in which donor cells formed retina. FIGS. 6H-J′ show retinas of tadpoles receiving donor cells expressing YFP alone (FIGS. 6H,H′), or in combination with Noggin (FIGS. 6I,I′), or Noggin with Tbx3MO (FIGS. 6J,J′). Sections were stained to detect cell nuclei (DAPI; blue), donor-derived cells (YFP; green), and neural tissue (Tubb2b; red). Eyes are oriented with dorsal side up. Error bars represent mean±s.e.m. P-values calculated using one-way ANOVA analysis: *** P≦0.001, **** P≦0.0001. Scale bar, 50 μm.

FIGS. 7A-7Z demonstrate that Tbx3 induces neural but not retinal tissue and is required for Noggin to determine pluripotent cells to a neural and retinal fate. FIGS. 7A-7O show donor animal cap cells that were isolated from embryos injected with the indicated mRNAs and morpholinos were transplanted to the flank of stage 15 embryos and grown to tadpole stages (ACT→Flank). Arrowheads in FIGS. 7A-7E indicate location of YFP positive transplant on the flank of tadpoles (green fluorescence, FIGS. 7A′-E′). Sections of transplanted cells are stained for the tracer YFP (FIGS. 7F-O), the neural marker Tubb2b (FIGS. 7F-J) and rod photoreceptor marker, XAP-2 (FIGS. 7 K-O). FIGS. 7P,Q are histograms showing the percent of donor transplants expressing YFP+/Tubb2b+ or YFP+/XAP-2+ in flank transplants. FIGS. 7R-7Z show eye fields isolated from stage 15 embryos injected in one dorsal blastomere at the 8-cell stage with YFP only, or in combination with CoMO or Tbx3MO were transplanted to the flank of stage 15 host embryos (EF→Flank). At stage 43 tadpoles (FIGS. 7R, 7S, and 7T) were sectioned and stained for YFP and Tubb2b (FIGS. 7U-W) or XAP-2 (FIGS. 7X-Z). All sections are also stained with DAPI to visualize cell nuclei. P-values are P≦0.05 (*), P≦0.01 (**), and P≦0.001 (***). Sale bars, 400 μm (FIGS. 7A-E and R-S), 50 μm (FIGS. 7F-O and U-Z).

FIGS. 8A-8E′ show magnified view of the tadpoles shown in FIGS. 7A-E. Animal cap cells isolated from embryos injected with the indicated mRNAs and morpholinos were transplanted to the flank of stage 15 embryos, which were then grown to tadpole stages (ACT→Flank) which are depicted in FIGS. 8A-8E′. Arrowheads (FIGS. 8A-E) indicate location of YFP positive (FIGS. 8A′-E′) transplant on the flank of tadpoles. Scale bar, 400 μm.

FIGS. 9A-9B demonstrate that Tbx3 knockdown generates cement gland in Noggin expressing donor cells. FIGS. 9A-B show transverse sections of stage 43 embryos with flank (FIG. 9A) and eye field (FIG. 9B) transplants of donor cells expressing mCherry, Noggin and Tbx3MO. Sections were stained to detect cell nuclei (DAPI; blue), donor-derived tissue (mCherry; red) and cement gland (ECL; green). Eye is oriented with dorsal side to the top. Scale bar, 50 μm.

FIGS. 10A-10N demonstrate that Tbx3 expressing cells are specified to a spinal cord, not retinal fate when transplanted to the posterior neural plate. In FIGS. 10A-10L, pluripotent cells isolated from embryos injected with the indicated mRNAs were transplanted to the posterior neural plate of stage 15 embryos and grown to tadpole stages (ACT→PNP) (tadpoles shown in FIGS. 10A-10C). Arrowheads (FIGS. 10A-C) indicate location of YFP positive donor tissue. FIGS. 10D-L show transverse sections of host stage 43 tadpoles that received transplants of pluripotent cells expressing YFP alone (FIGS. 10D,G,L), YFP with Noggin (FIGS. 10E,H,K) or YFP with Tbx3 (FIGS. 10F,I,L). Sections were stained for Tubb2b (orangish-red, FIGS. 10D-F) to detect neural tissue, XAP-2 (red, FIGS. 10G-I) for rod outer segments, Sox2 (magenta, FIGS. 10J-L) for ventricular zone, Islet-1/2 (yellow, FIGS. 10J-10L) for Rohon-Beard and motor neuron cells, DAPI (blue) for cell nuclei, and YFP (green) to mark donor derived tissues. FIG. 10M is a histogram showing the percent of host embryos with cells double-stained for YFP and Tubb2b (orange), XAP-2 (red), Sox2 (magenta), or Islet-1/2 (yellow) in mosaic spinal cords. Animal cap cells were isolated at stage 9 from embryos injected in both blastomeres at the 2-cell stage with YFP (500 pg), Tbx3 (50 pg), or Noggin (2.5 pg) as shown in FIG. 10N. Cells were cultured in vitro to the equivalent of stage 21 and RT-PCR was used to detecting expression of ncam1, tubb2b, t (xbra) and actc1. Histone H4 (h4) was used as a loading control. Controls included RNA isolated from whole embryos and processed with (WE) and without (WERT) reverse transcriptase. Scale bar, 400 μm (FIGS. 10A-C), 100 μm (FIGS. 10D-L).

FIGS. 11A-11D′″ show a magnified view of Noggin and Tbx3-treated transplanted cells expressing Sox2 or Islet1/2. FIGS. 11A-B show panels from FIGS. 10 K,L with a dashed white box depicting the area of magnification that is shown in FIGs C-C′″ and D-D′″. Each column of images were taken from the same sample. Ectodermal explants were isolated from embryos injected with YFP and Noggin (FIG. 11A) or YFP and Tbx3 RNA-injected embryos (FIG. 11B), transplanted to the posterior neural plate at stage 15 and the resulting tadpoles were sectioned and stained at stage 43 (ACT→PNP). FIGS. 11C-C′″ show enlarged images of the boxed area in FIG. 11A, and FIGs D-D′″ show enlarged images of the boxed area in FIG. 11 B. Sox2 positive cells co-expressing YFP are marked with the arrows, and Islet1/2 positive cells co-expressing YFP are marked with the arrowheads.

FIGS. 12A-12V demonstrate that Noggin and Tbx3 repress bmp4 expression in vitro and in vivo. In situ hybridization was used to detect changes in bmp4 expression in ectodermal explants and intact embryos. FIGS. 12A-12C and 12G-12N show ectodermal explants that were isolated from stage 9 embryos injected bilaterally at the 2-cell stage with mRNA of the indicated construct. Explants were left untreated (FIGS. 12A-12C) until stage 22, or treated from stage 15 with DMSO-only (FIGS. 12G-12J) or dexamethasone (FIGS. 12K-12N), then processed for bmp4 expression at stage 22 by in situ hybridization. FIGS. 12D-12F and 12O-12V show intact embryos that were injected unilaterally in one blastomere at the 4-cell stage with the indicated construct, grown to stage 9 and treated with hormone until stage 12.5, when they were processed by in situ hybridization to detect bmp4 expression. Amount of RNAs injected were: 500 pg YFP, 2.5 pg Noggin, 50 pg Tbx3, 100 pg Tbx3-GR, 250 pg DBD-EnR-GR, 5 pg VP16-DBDGR. Dorsal view, anterior toward the bottom. Scale bar, 400 μm.

FIGS. 13A-13F demonstrate that Tbx3 is necessary for the ability of Noggin to repress bmp4 expression in ectodermal explants. In situ hybridization was used to detect changes in bmp4 expression in the ectodermal explants depicted in FIGS. 13A-13F. Ectodermal explants were isolated from embryos injected bilaterally with mRNA at the 2-cell stage of the indicated construct and/or morpholino. In situ hybridization for bmp4 expression performed at the equivalent of stage 22. Embryos injected with 500 pg YFP, 2.5 pg Noggin, 20 ng morpholinos (per blastomere). Scale bar, 200 μm.

FIGS. 14A-14AA demonstrate that Tbx3 repressor activity is required at eye field stages for normal neural patterning and eye formation. FIGS. 14A-R show images of in situ hybridization used to detect changes in rax, pax6, otx2, foxg1 and ag1 transcript levels at embryonic stage 15. To target the anterior neural plate embryos were injected in one blastomere at the eight-cell stage with B-gal RNA alone (150 pg, FIGS. 14A-14F), and in combination with DBD-EnR-GR (50 pg, FIGS. 14G-14L) or VP16-DBD-GR (5 pg, FIGS. 14 M-14R) RNA. At stage 12.5, embryos were treated with DMSO-only (FIGS. 14 A,G,M) or DMSO containing dexamethasone (FIGS. 14 B-14F, 14H-14L and 14N-14R). Total number of embryos injected in two biological replicates, and the percentage showing a change in expression on the injected side are indicated in the lower left and right side of each panel, respectively. FIGS. 14S-AA demonstrate that the repressor activity of Tbx3 is required at eye field stages for normal eye formation. Control (FIGS. 14S-14V) and VP16-DBD-GR (FIGS. 14W-14Z) injected embryos were treated with DMSO only at stg. 12.5 (FIGS. 14S,14W) or containing dexamethasone starting at stage 12.5 (FIGS. 14T,14X), 15, (FIGS. 14U,14Y), 20 (FIGS. 14V, 14Z) and 24 (not shown). The total number of embryos treated in two biological replicates is indicated in the lower left side of each panel (FIGS. 14S-14Z). Histogram shows the percent of stage 43 tadpoles with the indicated eye defects (FIG. 14 AA). Scale bar is 300 μm (FIGS. 14A-14R), 400 μm (FIGS. 14S-14Z).

FIGS. 15A-15T demonstrate that Tbx3 knockdown results in progressive loss of donor eye field cells and their progeny during eye development. FIGS. 15A-15R show donor embryos that were injected into 1 dorsal blastomere at the 8-cell stage, then cultured to stage 15, when a portion of the donor eye fields from YFP-only (FIGS. 15A-15F, 500 pg, n=59), YFP plus CoMO (FIGS. 15G-15L, 10 ng, n=54), or YFP plus Tbx3MO (FIGS. 15M-15R, 10 ng, n=58) injected embryos were grafted into host, stage 15 eye fields (EF→EF). The fate of YFP positive donor cells was followed using brightfield (FIGS. 15A, 15G, 15M, insets 15C′-15E′, 15I′-15K′, and 15O′-15Q′) and YFP fluorescence (FIGS. 15B-15E, 15H-15K, and 15N-15Q) at stages 25, 35, 39 and 43. FIGS. 15F, 15L, and 15R show sections of stage 43 retinas that were stained for YFP-positive donor cells (green), the rod marker XAP-2 (red) and nuclei (blue). FIG. 15S shows the percent of live tadpoles with detectable YFP expression. FIG. 15T shows the volume of YFP-positive cells in retinas that received donor eye field transplants from YFP-only, YFP plus CoMO or YFP plus Tbx3MO transplants (YFP n=20, CoMO n=19, Tbx3MO n=20). Dotted lines indicate the boundary of the optic vesicle or cup. Error bars are standard error of the mean, *P≦0.05, and ****P≦0.0001. Scale bar, 50 μm F,L,R, and 200 μm all others.

FIGS. 16A-16N demonstrate that Tbx3 knockdown results in retinal progenitor apoptosis and eye defects. Eye field cells isolated from embryos expressing YFP (FIGS. 16A-16D′), CoMO (FIGS. 16E-16H′) or Tbx3MO-LS (FIGS. 161-16L′) were grafted into the eye field of untreated embryos (EF→EF). TUNEL staining was used to detect cell death of the transplanted (YFP-positive) cells at stage 22 (FIGS. 16A-166A′, 16E-16E′, 16I-16I′), 25 (FIGS. 16B-16B′, 16F-16F′, 16J-16J′), 35 (FIGS. 16C-16C′, 16G-16G′, 16K-16K′), and 39 (FIGS. 16D-16D′, 16H-16H′, 16L-16L′). Dotted lines indicate the outline of the optic vesicle (stgs. 22 and 25), optic cup and lens (stgs. 35 and 39). FIG. 16M is a line graph indicating the number of TUNEL positive donor (YFP-positive) cells per unit volume of transplanted cells as a function of developmental stage. FIG. 16N shows the number of TUNEL/YFP double-positive cells per unit volume that were detected in the stage 35 retina of tadpoles that received eye field transplants from YFP-only, CoMO, Tbx3MO-LS and Tbx3MO-SP injected embryos at stage 15. Dorsal retina is the top of each panel. Error bars are standard error of the mean, N=2; **P≦0.01, ***P≦0.001, and ****P≦0.0001. Scale bar, 50 μm.

FIGS. 17A-17V demonstrate that Tbx3 and Pax6 are sufficient in combination, for specification of pluripotent cells to a retinal fate. FIGS. 17A-17T show pluripotent cells isolated from embryos injected with the indicated mRNAs were transplanted to the flank of stage 15 embryos and grown to tadpoles (ACT→Flank). Arrowheads in FIGS. 17A-17E indicate location of YFP-positive transplant (green fluorescence, FIGS. 17A′-17E′). FIGS. 17F-17T show sections of transplanted cells stained for a neural marker (Tubb2b, orange), rod photoreceptor marker transducin (Gαt1, magenta) and nuclei (DAPI, blue). FIG. 17U shows percent of flank transplants with YFP+/Tubb2b+ and YFP+/Gαt1+ cells. Scale bars, 400 μm (FIGS. 17A-17E), 50 μm (FIGS. 17F-17T). FIG. 17V shows a schematic graphically illustrating a summary of results obtained from transplants performed in FIGS. 1A-1F, FIGS. 6A-6J′, FIGS. 7A-7T, FIGS. 10A-10N, and FIGS. 17A-17V).

FIGS. 18A-18C demonstrate that Noggin represses tbx3 expression in vitro, while inducing in vivo tbx3 expression. FIG. 18A shows results of RT-PCR used to detect changes in tbx3 expression in vitro at the equivalent of stages 12 and 15 in ectodermal explants isolated at stage 9 from YFP-only and YFP plus Noggin injected embryos. RT-PCR for histone h4 transcript was used to confirm approximately equal amounts of RNA was used in the reverse transcription reactions. FIGS. 18B-18C show whole mount in situ hybridization used to detect changes in tbx3 expression (violet) at stage 15 in response to injection of 3 gal-only (FIG. 18B; red) and 3 gal plus Noggin (FIG. 18C). Scale bar, 300 μm.

### DETAILED DESCRIPTION OF THE INVENTION

One aspect of the present invention relates to a method of producing an enriched preparation of neural progenitor cells from a population of pluripotent stem cells. The method comprises administering Tbx3 to the population of pluripotent stem cells and culturing the population of pluripotent stem cells, to which Tbx3 has been administered, under conditions suitable to produce the enriched preparation of neural progenitor cells from the population of pluripotent stem cells.

Neural progenitor cells are multipotent cells that have the capacity to create progeny that are more differentiated than them and yet retain the capacity to replenish the pool of progenitors. Neural progenitor cells are an intermediate cell type, arising from stem cells and generating progeny that are either neuronal cells (such as neuronal precursors or mature neurons) or glial cells (such as glial precursors, mature astrocytes, or mature oligodendrocytes). Neural progenitor cells are identified by their expression of one or more molecular markers, including, without limitation, the expression of CXCR4, Musashi, Nestin, Notch-1, SOX1, SOX2, SSEA-1 and Vimentin. Other molecular markers expressed by neural progenitor cells include Activin A, EAAT1/GLAST-1. EOMES, FABP7/B-FABP, IDS, NCAM-1/CD56, RPR2, and S100B.

An enriched preparation of neural progenitor cells, as referred to herein, is a preparation or population of cells comprising at least about 60% neural progenitor cells, at least 70% neural progenitor cells, 75% neural progenitor cells, 80% neural progenitor cells, or more, for example, about 85%, 90%, 95%, 96%, 97%, 98%, 99%, 100% neural progenitor cells.

The enriched preparation of neural progenitor cells as described herein is relatively devoid, e.g., containing less than 40, 30, 25, 20, 15, 10, 9, 8, 7, 6, 5, 4, 3, 2, or 1% of other cells types such as pluripotent stem cells, cells of a more differentiated lineage (e.g., neuronal progenitors, glial progenitors, retinal progenitors), or mature fully differentiated cells (e.g., neurons, astrocytes, oligodendrocytes). Contaminating cell types within the preparation of enriched neural progenitor cells can be identified based on their expression of cell specific molecular markers. For example, neuronal progenitor cells within the preparation can be identified by expression of neuronal progenitor specific markers, such as β-tubulin, neuron specific enolase (NSE), microtubule-associated protein-2 (MAP-2), and tyrosine hydroxylase. Likewise, differentiated neurons in the preparation can also be distinguished and identified based on their expression of NeuN, GAD, PSD-95, synaptophysin, and other markers known in the art. Cells of oligodendrocyte progenitor lineage can be identified by their expression of CD140a, SOX10, CD9 and NKX2.2, while differentiated oligodendrocytes can be identified by their expression of O1, O4 and myelin basic protein, or other oligodendrocyte-specific markers known in the art. Cells of the glial progenitor lineage can be identified by their expression of A2B5, astrocytes can be identified by their expression of GFAP, and microglia can be identified by their expression of CD11, CD32, and CD36. Accordingly, in one embodiment, the enriched preparation of neural progenitor cells is substantially or completely devoid of cells expressing these non-neural progenitor cell markers.

Differentiation is the process by which an unspecialized (“uncommitted”) or less specialized cell, e.g., a pluripotent stem cell, acquires the features of a more specialized cell, such as a neural progenitor cell. A differentiated or differentiation-induced cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

In accordance with this aspect of the invention, the neural progenitor cell preparation is produced from a population of pluripotent stem cells. Stem cells are undifferentiated cells defined by their ability at the single cell level to both self-renew and differentiate to produce progeny cells, including self-renewing progenitors, non-renewing progenitors, and terminally differentiated cells. Stem cells are also characterized by their ability to differentiate in vitro into functional cells of various ceil lineages from multiple germ layers (endoderm, mesoderm and ectoderm), as well as to give rise to tissues of multiple germ layers following transplantation and to contribute substantially to most, if not all, tissues following injection into blastocysts.

Stem cells are often categorized on the basis of the source from which they may be obtained. In one embodiment, the neural progenitor cell preparation is produced from a population of embryonic stem cells. Embryonic stem cells are pluripotent cells that are derived from the inner cell mass of a blastocyst-stage embryo. These cell types may be provided in the form of an established cell line, or they may be obtained directly from primary embryonic tissue and used immediately for differentiation. Exemplary embryonic stem cells include those listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)).

In another embodiment, the neural progenitor cell preparation is produced from a population of fetal stem cells. Fetal stem cells originate from tissues or membranes of a fetus, which in humans refers to the period from about six weeks of development to parturition. In another embodiment, the neural progenitor preparation is prepared from a population of postpartum stem cells. These stem cells are multipotent or pluripotent cells that originate substantially from extraembryonic tissue available after birth, namely, the placenta and the umbilicus. These cells have been found to possess features characteristic of pluripotent stem cells, including rapid proliferation and the potential for differentiation into many cell lineages. Postpartum stem cells may be blood-derived (e.g., as are those obtained from umbilical cord blood) or non-blood-derived (e.g., as obtained from the non-blood tissues of the umbilical cord and placenta). In yet another embodiment, the neural progenitor cell preparation is prepared from a population of adult neural stem cells.

In another embodiment, the neural progenitor cell preparation is produced from a population of induced pluripotent stem cells. Induced pluripotent stem cells are derived from non-pluripotent cells, such as somatic cells or tissue stem cells. For example, and without limitation, iPSCs can be derived from adult fibroblasts (see e.g., Streckfuss-Bomeke et al., “Comparative Study of Human-Induced Pluripotent Stem Cells Derived from Bone Marrow Cells, Hair Keratinocytes, and Skin Fibroblasts,” Eur. Heart J. doi: 10.1093/eurheartj/ehs203 (2012), which is hereby incorporated by reference in its entirety), umbilical cord blood (see e.g., Cai et al., “Generation of Human Induced Pluripotent Stem Cells from Umbilical Cord Matrix and Amniotic Membrane Mesenchymal Cells,” J. Biol. Chem. 285(15): 112227-11234 (2110) and Giorgetti et al., “Generation of Induced Pluripotent Stem Cells from Human Cord Blood Cells with only Two Factors: Oct4 and Sox2,” Nature Protocols, 5(4):811-820 (2010), which are hereby incorporated by reference in their entirety), bone marrow (see e.g., Streckfuss-Bomeke et al., “Comparative Study of Human-Induced Pluripotent Stem Cells Derived from Bone Marrow Cells, Hair Keratinocytes, and Skin Fibroblasts,” Eur. Heart J. doi: 10.1093/eurheartj/ehs203 (Jul. 12, 2012), and Hu et al., “Efficient Generation of Transgene-Free Induced Pluripotent Stem Cells from Normal and Neoplastic Bone Marrow and Cord Blood Mononuclear Cells,” Blood doi: 10.1182/blood-2010-07-298331 (Feb. 4, 2011) which are hereby incorporated by reference in their entirety), and peripheral blood (see e.g., Sommer et al., “Generation of Human Induced Pluripotent Stem Cells from Peripheral Blood using the STEMCCA Lentiviral Vector,” J. Vis. Exp. 68: e4327 doi: 10.3791/4327 (2012), which is hereby incorporated by reference in its entirety). iPSCs can also be derived from keratinocytes, mature B cells, mature T cells, pancreatic 3 cells, melanocytes, hepatocytes, foreskin cells, cheek cells, lung fibroblasts, myeloid progenitors, hematopoietic stem cells, adipose-derived stem cells, neural stem cells, and liver progenitor cells.

Induced pluripotent stem cells are produced by expressing a combination of reprogramming factors in a somatic cell. Suitable reprogramming factors that promote and induce iPSC generation include one or more of Oct4, Klf4, Sox2, c-Myc, Nanog, C/EBPa, Esrrb, Lin28, and Nr5a2. In certain embodiments, at least two reprogramming factors are expressed in a somatic cell to successfully reprogram the somatic cell. In other embodiments, at least three reprogramming factors are expressed in a somatic cell to successfully reprogram the somatic cell. In other embodiments, at least four reprogramming factors are expressed in a somatic cell to successfully reprogram the somatic cell.

iPSCs may be derived by methods known in the art including the use integrating viral vectors (e.g., lentiviral vectors, inducible lentiviral vectors, and retroviral vectors), excisable vectors (e.g., transposon and floxed lentiviral vectors), and non-integrating vectors (e.g., adenoviral and plasmid vectors) to deliver the genes that promote cell reprogramming (see e.g., Takahashi and Yamanaka, Cell 126:663-676 (2006); Okita et al., Nature 448:313-317 (2007); Nakagawa et al., Nat. Biotechnol. 26:101-106 (2007); Takahashi et al., Cell 131:1-12 (2007); Meissner et al. Nat. Biotech. 25:1177-1181 (2007); Yu et al. Science 318:1917-1920 (2007); Park et al. Nature 451:141-146 (2008); and U.S. Patent Application Publication No. 2008/0233610, which are hereby incorporated by reference in their entirety). Other methods for generating IPS cells include those disclosed in WO2007/069666, WO2009/006930, WO2009/006997, WO2009/007852, WO2008/118820, U.S. Patent Application Publication Nos. 2011/0200568 to Ikeda et al., 2010/0156778 to Egusa et al., 2012/0276070 to Musick, and 2012/0276636 to Nakagawa, Shi et al., Cell Stem Cell 3(5): 568-574 (2008), Kim et al., Nature 454: 646-650 (2008), Kim et al., Cell 136(3):411-419 (2009), Huangfu et al., Nature Biotech. 26: 1269-1275 (2008), Zhao et al., Cell Stem Cell 3: 475-479 (2008), Feng et al., Nature CellBiol. 11: 197-203 (2009), and Hanna et al., Cell 133(2): 250-264 (2008) which are hereby incorporated by reference in their entirety.

Integration free approaches, i.e., those using non-integrating and excisable vectors for deriving iPSCs free of transgenic sequences, are particularly suitable in the context of the present invention for therapeutic purposes. Suitable methods of iPSC production that utilize non-integrating vectors include methods that use adenoviral vectors (Stadtfeld et al., “Induced Pluripotent Stem Cells Generated without Viral Integration,” Science 322: 945-949 (2008), and Okita et al., “Generation of Mouse Induced Pluripotent Stem Cells without Viral Vectors,” Science 322: 949-953 (2008), which are hereby incorporated by reference in their entirety), Sendi virus vectors (Fusaki et al., “Efficient Induction of Transgene-Free Human Pluripotent Stem Cells Using a Vector Based on Sendi Virus, an RNA Virus That Does Not Integrate into the Host Genome,” Proc Jpn Acad. 85: 348-362 (2009), which is hereby incorporated by reference in its entirety), polycistronic minicircle vectors (Jia et al., “A Nonviral Minicircle Vector for Deriving Human iPS Cells,” Nat. Methods 7: 197-199 (2010), which is hereby incorporated by reference in its entirety), and self-replicating selectable episomes (Yu et al., “Human Induced Pluripotent Stem Cells Free of Vector and Transgene Sequences,” Science 324: 797-801 (2009), which is hereby incorporated by reference in its entirety). Suitable methods for iPSC generation using excisable vectors are described by Kaji et al., “Virus-Free Induction of Pluripotency and Subsequent Excision of Reprogramming Factors,” Nature 458: 771-775 (2009), Soldner et al., “Parkinson's Disease Patient-Derived Induced Pluripotent Stem Cells Free of Viral Reprogramming Factors,” Cell 136:964-977 (2009), Woltjen et al., “PiggyBac Transposition Reprograms Fibroblasts to Induced Pluripotent Stem Cells,” Nature 458: 766-770 (2009), and Yusa et al., “Generation of Transgene-Free Induced Pluripotent Mouse Stem Cells by the PiggyBac Transposon,” Nat. Methods 6: 363-369 (2009), which are hereby incorporated by reference in their entirety. Suitable methods for iPSC generation also include methods involving the direct delivery of reprogramming factors as recombinant proteins (Zhou et al., “Generation of Induced Pluripotent Stem Cells Using Recombinant Proteins,” Cell Stem Cell 4: 381-384 (2009), and Kim et al., “Generation of Human Induced Pluripotent Stem Cells by Direct Delivery of Reprogramming Proteins,” Cell Stem Cell 4: 472-476 (2009), which are hereby incorporated by reference in their entirety) or as whole-cell extracts isolated from ESCs (Cho et al., “Induction of Pluripotent Stem Cells from Adult Somatic Cells by Protein-Based Reprogramming without Genetic Manipulation,” Blood 116: 386-395 (2010), which is hereby incorporated by reference in its entirety).

The methods of iPSC generation described above can be modified to include small molecules that enhance reprogramming efficiency or even substitute for a reprogramming factor. These small molecules include, without limitation, epigenetic modulators such as the DNA methyltransferase inhibitor 5′-azacytidine, the histone deacetylase inhibitor VPA, and the G9a histone methyltransferase inhibitor BIX-01294 together with BayK8644, an L-type calcium channel agonist. Other small molecule reprogramming factors include those that target signal transduction pathways, such as TGF-β inhibitors and kinase inhibitors (e.g., kenpaullone) (see review by Sommer and Mostoslavsky, “Experimental Approaches for the Generation of Induced Pluripotent Stem Cells,” Stem Cell Res. Ther. 1:26 doi:10.1186/scrt26 (Aug. 10, 2010), which is hereby incorporated by reference in its entirety).

Suitable iPSCs derived from adult fibroblasts can be obtained following the procedure described in Streckfuss-Bomeke et al., “Comparative Study of Human-Induced Pluripotent Stem Cells Derived from Bone Marrow Cells, Hair Keratinocytes, and Skin Fibroblasts,” Eur. Heart J. doi: 10.1093/eurheartj/ehs203 (2012), which is hereby incorporated by reference in its entirety). iPSCs derived from umbilical cord blood cells can be obtained as described in Cai et al., “Generation of Human Induced Pluripotent Stem Cells from Umbilical Cord Matrix and Amniotic Membrane Mesenchymal Cells,” J. Biol. Chem. 285(15): 112227-11234 (2110) and Giorgetti et al., “Generation of Induced Pluripotent Stem Cells from Human Cord Blood Cells with only Two Factors: Oct4 and Sox2,” Nature Protocols, 5(4):811-820 (2010), which are hereby incorporated by reference in their entirety. iPSCs derived from bone marrow cells can be obtained using methods described in Streckfuss-Bomeke et al., “Comparative Study of Human-Induced Pluripotent Stem Cells Derived from Bone Marrow Cells, Hair Keratinocytes, and Skin Fibroblasts,” Eur. Heart J. doi: 10.1093/eurheartj/ehs203 (Jul. 12, 2012), and Hu et al., “Efficient Generation of Transgene-Free Induced Pluripotent Stem Cells from Normal and Neoplastic Bone Marrow and Cord Blood Mononuclear Cells,” Blood doi: 10.1182/blood-2010-07-298331 (Feb. 4, 2011) which are hereby incorporated by reference in their entirety). iPSCs derived from peripheral blood can be obtained following the methods described in Sommer et al., “Generation of Human Induced Pluripotent Stem Cells from Peripheral Blood using the STEMCCA Lentiviral Vector,” J. Vis. Exp. 68: e4327 doi:10.3791/4327 (2012), which is hereby incorporated by reference in its entirety. iPS cells contemplated for use in the methods of the present invention are not limited to those described in the above references, but rather includes cells prepared by any method as long as the cells have been artificially induced from cells other than pluripotent stem cells.

The source of the pluripotent stem cells, whether they are embryonic stem cells, fetal stem cells, iPSCs, etc., can be from any source, including mammalian sources, e.g., domesticated animals, such as cats and dogs; livestock (e.g., cattle, horses, pigs, sheep, and goats); laboratory animals (e.g., mice, rabbits, rats, and guinea pigs); non-human primates, and humans. Accordingly, in one embodiment, the preparation of neural progenitor cells is a preparation of mammalian neural progenitor cells. In one embodiment, the preparation of neural progenitor cells is a preparation of human neural progenitor cells.

The population of pluripotent stem cells can be propagated continuously in culture, using culture conditions that promote proliferation without promoting differentiation. Exemplary serum-containing stem cell medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM γ-mercaptoethanol. Just before use, human bFGF is added to 4 ng/mL (see WO 99/20741 to Geron Corp., which is hereby incorporated by reference in its entirety).

Pluripotent stem cells, such as embryonic stem cells can be cultured on a layer of feeder cells, typically fibroblasts derived from embryonic or fetal tissue. Alternatively these cells can be maintained in an undifferentiated state even without feeder cells.

Pluripotent stem cells are characterized by the expression of certain cell specific molecular markers, including for example, stage-specific embryonic antigen (SSEA)-3, SSEA-4, TRA-I-60, TRA-1-81, and alkaline phosphatase. Differentiation of the pluripotent stem cells in vitro into neural progenitor cells as described herein results in the loss of SSEA-4, Tra-1-60, and Tra-1-81 expression and increased expression of neural cell specific markers as described supra.

In accordance with this aspect of the present invention, T-box transcription factor Tbx3, is administered to the population of pluripotent stem cells to induce the differentiation of said stem cells to neural progenitor cells. Tbx3 is a member of a phylogenetically conserved family of genes that share a common DNA-binding domain, the T-box, and encode transcription factors involved in the regulation of developmental processes. This protein is a transcriptional repressor and is thought to play a role in the anterior/posterior axis of the tetrapod forelimb. Mutations in this gene cause ulnar-mammary syndrome, affecting limb, apocrine gland, tooth, hair, and genital development. As described in more detail herein, a new, previously unappreciated function of Tbx3 in cell differentiation has been discovered. Specifically, it has been discovered that Tbx3 is a repressor of bmp4 transcription, is sufficient for neural induction, and is required for the neural inducing activity of Noggin. Tbx3 alone is capable of inducing neural progenitor cell differentiation from a population of pluripotent stem cells.

Alternative splicing of the human Tbx3 gene (NCBI Reference Sequence NG_008315.1, which is hereby incorporated by reference in it entirety) result in three transcript variants encoding different isoforms. The first of these three sequence variants, NCBI Reference Sequence NM_005996.3 9 (which is hereby incorporated by reference in its entirety) (transcript variant 1), has the nucleotide sequence of SEQ ID NO: 1 as shown below.

| SEQ ID NO: 1-Tbx3 isoform 1 | |
|---|---|
| gaattctaga ggcggcggag ggtggcgagg agctctcgct ttctctcgct ccctccctct | 60 |
| ccgactccgt ctctctctct ctctctctct ctcccctccc tctctttccc tctgttccat | 120 |
| tttttccccc tctaaatcct ccctgccctg cgcgcctgga cacagattta ggaagcgaat | 180 |
| tcgctcacgt tttaggacaa ggaagagaga gaggcacggg agaagagccc agcaagattt | 240 |
| ggattgaaac cgagacaccc tccggaggct cggagcagag gaaggaggag gagggcggcg | 300 |
| aacggaagcc agtttgcaat tcaagttttg atagcgctgg tagaaggggg tttaaatcag | 360 |
| attttttttt ttttaaagga gagagacttt ttccgctctc tcgctccctg ttaaagccgg | 420 |
| gtctagcaca gctgcagacg ccaccagcga gaaagaggga gaggaagaca gatagggggc | 480 |
| gggggaagaa gaaaaagaaa ggtaaaaagt cttctaggag aacctttcac atttgcaaca | 540 |
| aaagacctag gggctggaga gagattcctg ggacgcaggg ctggagtgtc tatttcgagc | 600 |
| tcagcggcag ggctcgggcg cgagtcgaga ccctgctcgc tcctctcgct tctgaaaccg | 660 |
| acgttcagga gcggcttttt aaaaacgcaa ggcacaagga cggtcacccg cgcgactatg | 720 |
| tttgctgatt tttcgccttg ccctctttaa aagcggcctc ccattctcca aaagacactt | 780 |
| cccctcctcc ctttgaagtg cattagttgt gatttctgcc tccttttctt ttttctttct | 840 |
| tttttgtttt gctttttccc cccttttgaa ttatgtgctg ctgttaaaca acaacaaaaa | 900 |
| aacaacaaaa cacagcagct gcggacttgt ccccggctgg agcccagcgc cccgcctgga | 960 |
| gtggatgagc ctctccatga gagatccggt cattcctggg acaagcatgg cctaccatcc | 1020 |
| gttcctacct caccgggcgc cggacttcgc catgagcgcg gtgctgggtc accagccgcc | 1080 |
| gttcttcccc gcgctgacgc tgcctcccaa cggcgcggcg gcgctctcgc tgccgggcgc | 1140 |
| cctggccaag ccgatcatgg atcaattggt gggggcggcc gagaccggca tcccgttctc | 1200 |
| ctccctgggg ccccaggcgc atctgaggcc tttgaagacc atggagcccg aagaagaggt | 1260 |
| ggaggacgac cccaaggtgc acctggaggc taaagaactt tgggatcagt ttcacaagcg | 1320 |
| gggcaccgag atggtcatta ccaagtcggg aaggcgaatg tttcctccat ttaaagtgag | 1380 |
| atgttctggg ctggataaaa aagccaaata cattttattg atggacatta tagctgctga | 1440 |
| tgactgtcgt tataaatttc acaattctcg gtggatggtg gctggtaagg ccgaccccga | 1500 |
| aatgccaaag aggatgtaca ttcacccgga cagccccgct actggggaac agtggatgtc | 1560 |
| caaagtcgtc actttccaca aactgaaact caccaacaac atttcagaca aacatggatt | 1620 |
| tactatattg aactccatgc acaaatacca gccccggttc cacattgtaa gagccaatga | 1680 |
| catcttgaaa ctcccttata gtacatttcg gacatacttg ttccccgaaa ctgaattcat | 1740 |
| cgctgtgact gcataccaga atgataagat aacccagtta aaaatagaca acaacccttt | 1800 |
| tgcaaaaggt ttccgggaca ctggaaatgg ccgaagagaa aaaagaaaac agctcaccct | 1860 |
| gcagtccatg agggtgtttg atgaaagaca caaaaaggag aatgggacct ctgatgagtc | 1920 |
| ctccagtgaa caagcagctt tcaactgctt cgcccaggct tcttctccag ccgcctccac | 1980 |
| tgtagggaca tcgaacctca aagatttatg tcccagcgag ggtgagagcg acgccgaggc | 2040 |
| cgagagcaaa gaggagcatg gccccgaggc ctgcgacgcg gccaagatct ccaccaccac | 2100 |
| gtcggaggag ccctgccgtg acaagggcag ccccgcggtc aaggctcacc ttttcgctgc | 2160 |
| tgagcggccc cgggacagcg ggcggctgga caaagcgtcg cccgactcac gccatagccc | 2220 |
| cgccaccatc tcgtccagca ctcgcggcct gggcgcggag gagcgcagga gcccggttcg | 2280 |
| cgagggcaca gcgccggcca aggtggaaga ggcgcgcgcg ctcccgggca aggaggcctt | 2340 |
| cgcgccgctc acggtgcaga cggacgcggc cgccgcgcac ctggcccagg gccccctgcc | 2400 |
| tggcctcggc ttcgccccgg gcctggcggg ccaacagttc ttcaacgggc acccgctctt | 2460 |
| cctgcacccc agccagtttg ccatgggggg cgccttctcc agcatggcgg ccgctggcat | 2520 |
| gggtcccctc ctggccacgg tttctggggc ctccaccggt gtctcgggcc tggattccac | 2580 |
| ggccatggcc tctgccgctg cggcgcaggg actgtccggg gcgtccgcgg ccaccctgcc | 2640 |
| cttccacctc cagcagcacg tcctggcctc tcagggcctg gccatgtccc ctttcggaag | 2700 |
| cctgttccct tacccctaca cgtacatggc cgcagcggcg gccgcctcct ctgcggcagc | 2760 |
| ctccagctcg gtgcaccgcc accccttcct caatctgaac accatgcgcc cgcggctgcg | 2820 |
| ctacagcccc tactccatcc cggtgccggt cccggacggc agcagtctgc tcaccaccgc | 2880 |
| cctgccctcc atggcggcgg ccgcggggcc cctggacggc aaagtcgccg ccctggccgc | 2940 |
| cagcccggcc tcggtggcag tggactcggg ctctgaactc aacagccgct cctccacgct | 3000 |
| ctcctccagc tccatgtcct tgtcgcccaa actctgcgcg gagaaagagg cggccaccag | 3060 |
| cgaactgcag agcatccagc ggttggttag cggcttggaa gccaagccgg acaggtcccg | 3120 |
| cagcgcgtcc ccgtagaccc gtcccagaca cgtcttttca ttccagtcca gttcaggctg | 3180 |
| ccgtgcactt tgtcggatat aaaataaacc acgggcccgc catggcgtta gcccttcctt | 3240 |
| ttgcagttgc gtctgggaag gggccccgga ctccctcgag agaatgtgct agagacagcc | 3300 |
| cctgtcttct tggcgtggtt tatatgtccg ggatctggat cagattctgg gggctcagaa | 3360 |
| acgtcggttg cattgagcta ctgggggtag gagttccaac atttatgtcc agagcaactt | 3420 |
| ccagcaaggc tggtctgggt ctctgcccac caggcgggga ggtgttcaaa gacatctccc | 3480 |
| tcagtgcgga tttatatata tatttttcct tcactgtgtc aagtggaaac aaaaacaaaa | 3540 |
| tctttcaaaa aaaaaatcgg gacaagtgaa cacattaaca tgattctgtt tgtgcagatt | 3600 |
| aaaaacttta tagggacttg cattatcggt tctcaataaa ttactgagca gctttgtttg | 3660 |
| gggagggaag tccctaccat ccttgtttag tctatattaa gaaaatctgt gtctttttaa | 3720 |
| tattcttgtg atgttttcag agccgctgta ggtctcttct tgcatgtcca cagtaatgta | 3780 |
| tttgtggttt ttattttgaa cgcttgcttt tagagagaaa acaatatagc cccctaccct | 3840 |
| tttcccaatc ctttgccctc aaatcagtga cccaagggag ggggggattt aaagggaagg | 3900 |
| agtgggcaaa acacataaaa tgaatttatt atatctaagc tctgtagcag gattcatgtc | 3960 |
| gttctttgac agttctttct ctttcctgta tatgcaataa caaggtttta aaaaaataat | 4020 |
| aaagaagtga gactattaga caaagtattt atgtaattat ttgataactc ttgtaaatag | 4080 |
| gtggaatatg aatgcttgga aaattaaact ttaatttatt gacattgtac atagctctgt | 4140 |
| gtaaatagaa ttgcaactgt caggttttgt gttcttgttt tcctttagtt gggtttattt | 4200 |
| ccaggtcaca gaattgctgt taacactaga aaacacactt cctgcaccaa caccaatacc | 4260 |
| ctttcaaaag agttgtctgc aacatttttg ttttcttttt taatgtccaa aagtggggga | 4320 |
| aagtgctatt tcctattttc accaaaattg gggaaggagt gccactttcc agctccactt | 4380 |
| caaattcctt aaaatataac tgagattgct gtggggaggg aggagggcag aggctgcggt | 4440 |
| ttgacttttt aatttttctt ttgttatttg tatttgctag tctctgattt cctcaaaacg | 4500 |
| aagtggaatt tactactgtt gtcagtatcg gtgttttgaa ttggtgcctg cctatagaga | 4560 |
| tatattcaca gttcaaaagt caggtgctga gagatggttt aaagacaaat tcatgaaggt | 4620 |
| atattttgtg ttatagttgt tgatgagttc tttggttttc tgtatttttc cccctctctt | 4680 |
| taaaacatca ctgaaatttc aataaatttt tattgaaatg tctaaaaaaa aaaaaaaaaa | 4740 |
| aaaaaaaaaa aaaa | 4754 |

which is translated into the amino acid sequence of SEQ ID NO: 2 (NCBI Reference Sequence NP_005987.3; UniProtKB identifier 015119-1):

```
SEQ ID NO: 2-Tbx3 isoform 1
Met Ser Leu Ser Met Arg Asp Pro Val Ile Pro Gly Thr Ser Met Ala
1 5 10 15
Tyr His Pro Phe Leu Pro His Arg Ala Pro Asp Phe Ala Met Ser Ala
                        20 25 30
Val Leu Gly His Gln Pro Pro Phe Phe Pro Ala Leu Thr Leu Pro Pro
                35 40 45
Asn Gly Ala Ala Ala Leu Ser Leu Pro Gly Ala Leu Ala Lys Pro Ile
        50 55 60
Met Asp Gln Leu Val Gly Ala Ala Glu Thr Gly Ile Pro Phe Ser Ser
65 70 75 80
Leu Gly Pro Gln Ala His Leu Arg Pro Leu Lys Thr Met Glu Pro Glu
                                85 90 95
Glu Glu Val Glu Asp Asp Pro Lys Val His Leu Glu Ala Lys Glu Leu
                        100 105 110
Trp Asp Gln Phe His Lys Arg Gly Thr Glu Met Val Ile Thr Lys Ser
                115 120 125
Gly Arg Arg Met Phe Pro Pro Phe Lys Val Arg Cys Ser Gly Leu Asp
        130 135 140
Lys Lys Ala Lys Tyr Ile Leu Leu Met Asp Ile Ile Ala Ala Asp Asp
145 150 155 160
Cys Arg Tyr Lys Phe His Asn Ser Arg Trp Met Val Ala Gly Lys Ala
                                165 170 175
Asp Pro Glu Met Pro Lys Arg Met Tyr Ile His Pro Asp Ser Pro Ala
                        180 185 190
Thr Gly Glu Gln Trp Met Ser Lys Val Val Thr Phe His Lys Leu Lys
                195 200 205
Leu Thr Asn Asn Ile Ser Asp Lys His Gly Phe Thr Ile Leu Asn Ser
        210 215 220
Met His Lys Tyr Gln Pro Arg Phe His Ile Val Arg Ala Asn Asp Ile
225 230 235 240
Leu Lys Leu Pro Tyr Ser Thr Phe Arg Thr Tyr Leu Phe Pro Glu Thr
                                245 250 255
Glu Phe Ile Ala Val Thr Ala Tyr Gln Asn Asp Lys Ile Thr Gln Leu
                        260 265 270
Lys Ile Asp Asn Asn Pro Phe Ala Lys Gly Phe Arg Asp Thr Gly Asn
                275 280 285
Gly Arg Arg Glu Lys Arg Lys Gln Leu Thr Leu Gln Ser Met Arg Val
        290 295 300
Phe Asp Glu Arg His Lys Lys Glu Asn Gly Thr Ser Asp Glu Ser Ser
305 310 315 320
Ser Glu Gln Ala Ala Phe Asn Cys Phe Ala Gln Ala Ser Ser Pro Ala
                                325 330 335
Ala Ser Thr Val Gly Thr Ser Asn Leu Lys Asp Leu Cys Pro Ser Glu
                        340 345 350
Gly Glu Ser Asp Ala Glu Ala Glu Ser Lys Glu Glu His Gly Pro Glu
                355 360 365
Ala Cys Asp Ala Ala Lys Ile Ser Thr Thr Thr Ser Glu Glu Pro Cys
        370 375 380
Arg Asp Lys Gly Ser Pro Ala Val Lys Ala His Leu Phe Ala Ala Glu
385 390 395 400
Arg Pro Arg Asp Ser Gly Arg Leu Asp Lys Ala Ser Pro Asp Ser Arg
                                405 410 415
His Ser Pro Ala Thr Ile Ser Ser Ser Thr Arg Gly Leu Gly Ala Glu
                        420 425 430
Glu Arg Arg Ser Pro Val Arg Glu Gly Thr Ala Pro Ala Lys Val Glu
                435 440 445
Glu Ala Arg Ala Leu Pro Gly Lys Glu Ala Phe Ala Pro Leu Thr Val
        450 455 460
Gln Thr Asp Ala Ala Ala Ala His Leu Ala Gln Gly Pro Leu Pro Gly
465 470 475 480
Leu Gly Phe Ala Pro Gly Leu Ala Gly Gln Gln Phe Phe Asn Gly His
                                485 490 495
Pro Leu Phe Leu His Pro Ser Gln Phe Ala Met Gly Gly Ala Phe Ser
                        500 505 510
Ser Met Ala Ala Ala Gly Met Gly Pro Leu Leu Ala Thr Val Ser Gly
                515 520 525
Ala Ser Thr Gly Val Ser Gly Leu Asp Ser Thr Ala Met Ala Ser Ala
        530 535 540
Ala Ala Ala Gln Gly Leu Ser Gly Ala Ser Ala Ala Thr Leu Pro Phe
545 550 555 560
His Leu Gln Gln His Val Leu Ala Ser Gln Gly Leu Ala Met Ser Pro
                                565 570 575
Phe Gly Ser Leu Phe Pro Tyr Pro Tyr Thr Tyr Met Ala Ala Ala Ala
                        580 585 590
Ala Ala Ser Ser Ala Ala Ala Ser Ser Ser Val His Arg His Pro Phe
                595 600 605
Leu Asn Leu Asn Thr Met Arg Pro Arg Leu Arg Tyr Ser Pro Tyr Ser
        610 615 620
Ile Pro Val Pro Val Pro Asp Gly Ser Ser Leu Leu Thr Thr Ala Leu
625 630 635 640
Pro Ser Met Ala Ala Ala Ala Gly Pro Leu Asp Gly Lys Val Ala Ala
                                645 650 655
Leu Ala Ala Ser Pro Ala Ser Val Ala Val Asp Ser Gly Ser Glu Leu
                      660 665 670
Asn Ser Arg Ser Ser Thr Leu Ser Ser Ser Ser Met Ser Leu Ser Pro
                675 680 685
Lys Leu Cys Ala Glu Lys Glu Ala Ala Thr Ser Glu Leu Gln Ser Ile
        690 695 700
Gln Arg Leu Val Ser Gly Leu Glu Ala Lys Pro Asp Arg Ser Arg Ser
705 710 715 720
Ala Ser Pro
```

The second Tbx3 isoform, i.e., NCBI Reference Sequence NM_016569.3 (transcript variant 2), has the following nucleotide sequence of SEQ ID NO: 3 as shown below.

| SEQ ID NO: 3 | |
|---|---|
| Tbx 3 isoform 2 | |
| gaattctaga ggcggcggag ggtggcgagg agctctcgct ttctctcgct ccctccctct | 60 |
| ccgactccgt ctctctctct ctctctctct ctcccctccc tctctttccc tctgttccat | 120 |
| tttttccccc tctaaatcct ccctgccctg cgcgcctgga cacagattta ggaagcgaat | 180 |
| tcgctcacgt tttaggacaa ggaagagaga gaggcacggg agaagagccc agcaagattt | 240 |
| ggattgaaac cgagacaccc tccggaggct cggagcagag gaaggaggag gagggcggcg | 300 |
| aacggaagcc agtttgcaat tcaagttttg atagcgctgg tagaaggggg tttaaatcag | 360 |
| attttttttt ttttaaagga gagagacttt ttccgctctc tcgctccctg ttaaagccgg | 420 |
| gtctagcaca gctgcagacg ccaccagcga gaaagaggga gaggaagaca gatagggggc | 480 |
| gggggaagaa gaaaaagaaa ggtaaaaagt cttctaggag aacctttcac atttgcaaca | 540 |
| aaagacctag gggctggaga gagattcctg ggacgcaggg ctggagtgtc tatttcgagc | 600 |
| tcagcggcag ggctcgggcg cgagtcgaga ccctgctcgc tcctctcgct tctgaaaccg | 660 |
| acgttcagga gcggcttttt aaaaacgcaa ggcacaagga cggtcacccg cgcgactatg | 720 |
| tttgctgatt tttcgccttg ccctctttaa aagcggcctc ccattctcca aaagacactt | 780 |
| cccctcctcc ctttgaagtg cattagttgt gatttctgcc tccttttctt ttttctttct | 840 |
| tttttgtttt gctttttccc cccttttgaa ttatgtgctg ctgttaaaca acaacaaaaa | 900 |
| aacaacaaaa cacagcagct gcggacttgt ccccggctgg agcccagcgc cccgcctgga | 960 |
| gtggatgagc ctctccatga gagatccggt cattcctggg acaagcatgg cctaccatcc | 1020 |
| gttcctacct caccgggcgc cggacttcgc catgagcgcg gtgctgggtc accagccgcc | 1080 |
| gttcttcccc gcgctgacgc tgcctcccaa cggcgcggcg gcgctctcgc tgccgggcgc | 1140 |
| cctggccaag ccgatcatgg atcaattggt gggggcggcc gagaccggca tcccgttctc | 1200 |
| ctccctgggg ccccaggcgc atctgaggcc tttgaagacc atggagcccg aagaagaggt | 1260 |
| ggaggacgac cccaaggtgc acctggaggc taaagaactt tgggatcagt ttcacaagcg | 1320 |
| gggcaccgag atggtcatta ccaagtcggg aaggcgaatg tttcctccat ttaaagtgag | 1380 |
| atgttctggg ctggataaaa aagccaaata cattttattg atggacatta tagctgctga | 1440 |
| tgactgtcgt tataaatttc acaattctcg gtggatggtg gctggtaagg ccgaccccga | 1500 |
| aatgccaaag aggatgtaca ttcacccgga cagccccgct actggggaac agtggatgtc | 1560 |
| caaagtcgtc actttccaca aactgaaact caccaacaac atttcagaca aacatggatt | 1620 |
| tactttggcc ttcccaagtg atcacgctac gtggcagggg aattatagtt ttggtactca | 1680 |
| gactatattg aactccatgc acaaatacca gccccggttc cacattgtaa gagccaatga | 1740 |
| catcttgaaa ctcccttata gtacatttcg gacatacttg ttccccgaaa ctgaattcat | 1800 |
| cgctgtgact gcataccaga atgataagat aacccagtta aaaatagaca acaacccttt | 1860 |
| tgcaaaaggt ttccgggaca ctggaaatgg ccgaagagaa aaaagaaaac agctcaccct | 1920 |
| gcagtccatg agggtgtttg atgaaagaca caaaaaggag aatgggacct ctgatgagtc | 1980 |
| ctccagtgaa caagcagctt tcaactgctt cgcccaggct tcttctccag ccgcctccac | 2040 |
| tgtagggaca tcgaacctca aagatttatg tcccagcgag ggtgagagcg acgccgaggc | 2100 |
| cgagagcaaa gaggagcatg gccccgaggc ctgcgacgcg gccaagatct ccaccaccac | 2160 |
| gtcggaggag ccctgccgtg acaagggcag ccccgcggtc aaggctcacc ttttcgctgc | 2220 |
| tgagcggccc cgggacagcg ggcggctgga caaagcgtcg cccgactcac gccatagccc | 2280 |
| cgccaccatc tcgtccagca ctcgcggcct gggcgcggag gagcgcagga gcccggttcg | 2340 |
| cgagggcaca gcgccggcca aggtggaaga ggcgcgcgcg ctcccgggca aggaggcctt | 2400 |
| cgcgccgctc acggtgcaga cggacgcggc cgccgcgcac ctggcccagg gccccctgcc | 2460 |
| tggcctcggc ttcgccccgg gcctggcggg ccaacagttc ttcaacgggc acccgctctt | 2520 |
| cctgcacccc agccagtttg ccatgggggg cgccttctcc agcatggcgg ccgctggcat | 2580 |
| gggtcccctc ctggccacgg tttctggggc ctccaccggt gtctcgggcc tggattccac | 2640 |
| ggccatggcc tctgccgctg cggcgcaggg actgtccggg gcgtccgcgg ccaccctgcc | 2700 |
| cttccacctc cagcagcacg tcctggcctc tcagggcctg gccatgtccc ctttcggaag | 2760 |
| cctgttccct tacccctaca cgtacatggc cgcagcggcg gccgcctcct ctgcggcagc | 2820 |
| ctccagctcg gtgcaccgcc accccttcct caatctgaac accatgcgcc cgcggctgcg | 2880 |
| ctacagcccc tactccatcc cggtgccggt cccggacggc agcagtctgc tcaccaccgc | 2940 |
| cctgccctcc atggcggcgg ccgcggggcc cctggacggc aaagtcgccg ccctggccgc | 3000 |
| cagcccggcc tcggtggcag tggactcggg ctctgaactc aacagccgct cctccacgct | 3060 |
| ctcctccagc tccatgtcct tgtcgcccaa actctgcgcg gagaaagagg cggccaccag | 3120 |
| cgaactgcag agcatccagc ggttggttag cggcttggaa gccaagccgg acaggtcccg | 3180 |
| cagcgcgtcc ccgtagaccc gtcccagaca cgtcttttca ttccagtcca gttcaggctg | 3240 |
| ccgtgcactt tgtcggatat aaaataaacc acgggcccgc catggcgtta gcccttcctt | 3300 |
| ttgcagttgc gtctgggaag gggccccgga ctccctcgag agaatgtgct agagacagcc | 3360 |
| cctgtcttct tggcgtggtt tatatgtccg ggatctggat cagattctgg gggctcagaa | 3420 |
| acgtcggttg cattgagcta ctgggggtag gagttccaac atttatgtcc agagcaactt | 3480 |
| ccagcaaggc tggtctgggt ctctgcccac caggcgggga ggtgttcaaa gacatctccc | 3540 |
| tcagtgcgga tttatatata tatttttcct tcactgtgtc aagtggaaac aaaaacaaaa | 3600 |
| tctttcaaaa aaaaaatcgg gacaagtgaa cacattaaca tgattctgtt tgtgcagatt | 3660 |
| aaaaacttta tagggacttg cattatcggt tctcaataaa ttactgagca gctttgtttg | 3720 |
| gggagggaag tccctaccat ccttgtttag tctatattaa gaaaatctgt gtctttttaa | 3780 |
| tattcttgtg atgttttcag agccgctgta ggtctcttct tgcatgtcca cagtaatgta | 3840 |
| tttgtggttt ttattttgaa cgcttgcttt tagagagaaa acaatatagc cccctaccct | 3900 |
| tttcccaatc ctttgccctc aaatcagtga cccaagggag ggggggattt aaagggaagg | 3960 |
| agtgggcaaa acacataaaa tgaatttatt atatctaagc tctgtagcag gattcatgtc | 4020 |
| gttctttgac agttctttct ctttcctgta tatgcaataa caaggtttta aaaaaataat | 4080 |
| aaagaagtga gactattaga caaagtattt atgtaattat ttgataactc ttgtaaatag | 4140 |
| gtggaatatg aatgcttgga aaattaaact ttaatttatt gacattgtac atagctctgt | 4200 |
| gtaaatagaa ttgcaactgt caggttttgt gttcttgttt tcctttagtt gggtttattt | 4260 |
| ccaggtcaca gaattgctgt taacactaga aaacacactt cctgcaccaa caccaatacc | 4320 |
| ctttcaaaag agttgtctgc aacatttttg ttttcttttt taatgtccaa aagtggggga | 4380 |
| aagtgctatt tcctattttc accaaaattg gggaaggagt gccactttcc agctccactt | 4440 |
| caaattcctt aaaatataac tgagattgct gtggggaggg aggagggcag aggctgcggt | 4500 |
| ttgacttttt aatttttctt ttgttatttg tatttgctag tctctgattt cctcaaaacg | 4560 |
| aagtggaatt tactactgtt gtcagtatcg gtgttttgaa ttggtgcctg cctatagaga | 4620 |
| tatattcaca gttcaaaagt caggtgctga gagatggttt aaagacaaat tcatgaaggt | 4680 |
| atattttgtg ttatagttgt tgatgagttc tttggttttc tgtatttttc cccctctctt | 4740 |
| taaaacatca ctgaaatttc aataaatttt tattgaaatg tctaaaaaaa aaaaaaaaaa | 4800 |
| aaaaaaaaaa aaaa | 4814 |

which is translated into the amino acid sequence of SEQ ID NO: 4 (NCBI Reference Sequence NP_0057653.3; UniProtKB identifier 015119-2).

```
SEQ ID NO: 4
Tbx isoforrn 2
Met Ser Leu Ser Met Arg Asp Pro Val Ile Pro Gly Thr Ser Met Ala
1 5 10 15
Tyr His Pro Phe Leu Pro His Arg Ala Pro Asp Phe Ala Met Ser Ala
                        20 25 30
Val Leu Gly His Gln Pro Pro Phe Phe Pro Ala Leu Thr Leu Pro Pro
                35 40 45
Asn Gly Ala Ala Ala Leu Ser Leu Pro Gly Ala Leu Ala Lys Pro Ile
        50 55 60
Met Asp Gln Leu Val Gly Ala Ala Glu Thr Gly Ile Pro Phe Ser Ser
65 70 75 80
Leu Gly Pro Gln Ala His Leu Arg Pro Leu Lys Thr Met Glu Pro Glu
                                85 90 95
Glu Glu Val Glu Asp Asp Pro Lys Val His Leu Glu Ala Lys Glu Leu
                        100 105 110
Trp Asp Gln Phe His Lys Arg Gly Thr Glu Met Val Ile Thr Lys Ser
                115 120 125
Gly Arg Arg Met Phe Pro Pro Phe Lys Val Arg Cys Ser Gly Leu Asp
        130 135 140
Lys Lys Ala Lys Tyr Ile Leu Leu Met Asp Ile Ile Ala Ala Asp Asp
145 150 155 160
Cys Arg Tyr Lys Phe His Asn Ser Arg Trp Met Val Ala Gly Lys Ala
                                165 170 175
Asp Pro Glu Met Pro Lys Arg Met Tyr Ile His Pro Asp Ser Pro Ala
                        180 185 190
Thr Glu Gln Trp Met Ser Lys Val Val Thr Phe His Lys Leu Lys Leu
                195 200 205
Thr Asn Asn Ile Ser Asp Lys His Gly Phe Thr Leu Ala Phe Pro Ser
        210 215 220
Asp His Ala Thr Trp Gln Gly Asn Tyr Ser Phe Gly Thr Gln Thr Ile
225 230 235 240
Leu Asn Ser Met His Lys Tyr Gln Pro Arg Phe His Ile Val Arg Ala
                                245 250 255
Asn Asp Ile Leu Lys Leu Pro Tyr Ser Thr Phe Arg Thr Tyr Leu Phe
                        260 265 270
Pro Glu Thr Glu Phe Ile Ala Val Thr Ala Tyr Gln Asn Asp Lys Ile
                275 280 285
Thr Gln Leu Lys Ile Asp Asn Asn Pro Phe Ala Lys Gly Phe Arg Asp
        290 295 300
Thr Gly Asn Gly Arg Arg Glu Lys Arg Lys Gln Leu Thr Leu Gln Ser
305 310 315 320
Met Arg Val Phe Asp Glu Arg His Lys Lys Glu Asn Gly Thr Ser Asp
                                325 330 335
Glu Ser Ser Ser Glu Gln Ala Ala Phe Asn Cys Phe Ala Gln Ala Ser
                        340 345 350
Ser Pro Ala Ala Ser Thr Val Gly Thr Ser Asn Leu Lys Asp Leu Cys
                355 360 365
Pro Ser Glu Gly Glu Ser Asp Ala Glu Ala Glu Ser Lys Glu Glu His
        370 375 380
Gly Pro Glu Ala Cys Asp Ala Ala Lys Ile Ser Thr Thr Thr Ser Glu
385 390 395 400
Glu Pro Cys Arg Asp Lys Gly Ser Pro Ala Val Lys Ala His Leu Phe
                                405 410 415
Ala Ala Glu Arg Pro Arg Asp Ser Gly Arg Leu Asp Lys Ala Ser Pro
                        420 425 430
Asp Ser Arg His Ser Pro Ala Thr Ile Ser Ser Ser Thr Arg Gly Leu
                435 440 445
Gly Ala Glu Glu Arg Arg Ser Pro Val Arg Glu Gly Thr Ala Pro Ala
        450 455 460
Lys Val Glu Glu Ala Arg Ala Leu Pro Gly Lys Glu Ala Phe Ala Pro
465 470 475 480
Leu Thr Val Gln Thr Asp Ala Ala Ala Ala His Leu Ala Gln Gly Pro
                                485 490 495
Leu Pro Gly Leu Gly Phe Ala Pro Gly Leu Ala Gly Gln Gln Phe Phe
                        500 505 510
Asn Gly His Pro Leu Phe Leu His Pro Ser Gln Phe Ala Met Gly Gly
                515 520 525
Ala Phe Ser Ser Met Ala Ala Ala Gly Met Gly Pro Leu Leu Ala Thr
        530 535 540
Val Ser Gly Ala Ser Thr Gly Val Ser Gly Leu Asp Ser Thr Ala Met
545 550 555 560
Ala Ser Ala Ala Ala Ala Gln Gly Leu Ser Gly Ala Ser Ala Ala Thr
                                565 570 575
Leu Pro Phe His Leu Gln Gln His Val Leu Ala Ser Gln Gly Leu Ala
                        580 585 590
Met Ser Pro Phe Gly Ser Leu Phe Pro Tyr Pro Tyr Thr Tyr Met Ala
                595 600 605
Ala Ala Ala Ala Ala Ser Ser Ala Ala Ala Ser Ser Ser Val His Arg
        610 615 620
His Pro Phe Leu Asn Leu Asn Thr Met Arg Pro Arg Leu Arg Tyr Ser
625 630 635 640
Pro Tyr Ser Ile Pro Val Pro Val Pro Asp Gly Ser Ser Leu Leu Thr
                                645 650 655
Thr Ala Leu Pro Ser Met Ala Ala Ala Ala Gly Pro Leu Asp Gly Lys
                        660 665 670
Val Ala Ala Leu Ala Ala Ser Pro Ala Ser Val Ala Val Asp Ser Gly
                675 680 685
Ser Glu Leu Asn Ser Arg Ser Ser Thr Leu Ser Ser Ser Ser Met Ser
        690 695 700
Leu Ser Pro Lys Leu Cys Ala Glu Lys Glu Ala Ala Thr Ser Glu Leu
705 710 715 720
Gln Ser Ile Gln Arg Leu Val Ser Gly Leu Glu Ala Lys Pro Asp Arg
                                725 730 735
Ser Arg Ser Ala Ser Pro
                        740
```

A third Tbx3 isoform, i.e., UniProtKB accession number 015119-3, has an amino acid sequence of SEQ ID NO: 5 as shown below.

```
SEQ ID NO: 5
Tbx3 isoform 3
Met Ser Leu Ser Met Arg Asp Pro Val Ile Pro Gly Thr Ser Met Ala
1 5 10 15
Tyr His Pro Phe Leu Pro His Arg Ala Pro Asp Phe Ala Met Ser Ala
                        20 25 30
Val Leu Gly His Gln Pro Pro Phe Phe Pro Ala Leu Thr Leu Pro Pro
                35 40 45
Asn Gly Ala Ala Ala Leu Ser Leu Pro Gly Ala Leu Ala Lys Pro Ile
        50 55 60
Met Asp Gln Leu Val Gly Ala Ala Glu Thr Gly Ile Pro Phe Ser Ser
65 70 75 80
Leu Gly Pro Gln Ala His Leu Arg Pro Leu Lys Thr Met Glu Pro Glu
                                85 90 95
Glu Glu Val Glu Asp Asp Pro Lys Val His Leu Glu Ala Lys Glu Leu
                        100 105 110
Trp Asp Gln Phe His Lys Arg Gly Thr Glu Met Val Ile Thr Lys Ser
                115 120 125
Gly Arg Arg Met Phe Pro Pro Phe Lys Val Arg Cys Ser Gly Leu Asp
        130 135 140
Lys Lys Ala Lys Tyr Ile Leu Leu Met Asp Ile Ile Ala Ala Asp Asp
145 150 155 160
Cys Arg Tyr Lys Phe His Asn Ser Arg Trp Met Val Ala Gly Lys Ala
                                165 170 175
Asp Pro Glu Met Pro Lys Arg Met Tyr Ile His Pro Asp Ser Pro Ala
                        180 185 190
Thr Gly Glu Gln Trp Met Ser Lys Val Val Thr Phe His Lys Leu Lys
                195 200 205
Leu Thr Asn Asn Ile Ser Asp Lys His Gly Phe Thr Leu Ala Phe Pro
        210 215 220
Ser Asp His Ala Thr Trp Gln Gly Asn Tyr Ser Phe Gly Thr Gln Thr
225 230 235 240
Ile Leu Asn Ser Met His Lys Tyr Gln Pro Arg Phe His Ile Val Arg
                                245 250 255
Ala Asn Asp Ile Leu Lys Leu Pro Tyr Ser Thr Phe Arg Thr Tyr Leu
                        260 265 270
Phe Pro Glu Thr Glu Phe Ile Ala Val Thr Ala Tyr Gln Asn Asp Lys
                275 280 285
Ile Thr Gln Leu Lys Ile Asp Asn Asn Pro Phe Ala Lys Gly Phe Arg
        290 295 300
Asp Thr Gly Asn Gly Arg Arg Glu Lys Arg Lys Gln Leu Thr Leu Gln
305 310 315 320
Ser Met Arg Val Phe Asp Glu Arg His Lys Lys Glu Asn Gly Thr Ser
                                325 330 335
Asp Glu Ser Ser Ser Glu Gln Ala Ala Phe Asn Cys Phe Ala Gln Ala
                        340 345 350
Ser Ser Pro Ala Ala Ser Thr Val Gly Thr Ser Asn Leu Lys Asp Leu
                355 360 365
Cys Pro Ser Glu Gly Glu Ser Asp Ala Glu Ala Glu Ser Lys Glu Glu
        370 375 380
His Gly Pro Glu Ala Cys Asp Ala Ala Lys Ile Ser Thr Thr Thr Ser
385 390 395 400
Glu Glu Pro Cys Arg Asp Lys Gly Ser Pro Ala Val Lys Ala His Leu
                                405 410 415
Phe Ala Ala Glu Arg Pro Arg Asp Ser Gly Arg Leu Asp Lys Ala Ser
                        420 425 430
Pro Asp Ser Arg His Ser Pro Ala Thr Ile Ser Ser Ser Thr Arg Gly
                435 440 445
Leu Gly Ala Glu Glu Arg Arg Ser Pro Val Arg Glu Gly Thr Ala Pro
        450 455 460
Ala Lys Val Glu Glu Ala Arg Ala Leu Pro Gly Lys Glu Ala Phe Ala
465 470 475 480
Pro Leu Thr Val Gln Thr Asp Ala Ala Ser Ala Ala Ala Ser Ser Ser
                                485 490 495
Val His Arg His Pro Phe Leu Asn Leu Asn Thr Met Arg Pro Arg Leu
                        500 505 510
Arg Tyr Ser Pro Tyr Ser Ile Pro Val Pro Val Pro Asp Gly Ser Ser
                515 520 525
Leu Leu Thr Thr Ala Leu Ala Ala Ser Pro Ala Ser Val Ala Val Asp
        530 535 540
Ser Gly Ser Glu Leu Asn Ser Arg Ser Ser Thr Leu Ser Ser Ser Ser
545 550 555 560
Met Ser Leu Ser Pro Lys Leu Cys Ala Glu Lys Glu Ala Ala Thr Ser
                                565 570 575
Glu Leu Gln Ser Ile Gln Arg Leu Val Ser Gly Leu Glu Ala Lys Pro
                        580 585 590
Asp Arg Ser Arg Ser Ala Ser Pro
                595 600
```

Mammalian homologs of Tbx3 have been described for Bos taurus (NCBI Reference Sequence XP_001787873.1, which sequence information is hereby incorporated by reference in its entirety), Mus musculus (NCBI Reference Sequence NP_035665.2, which sequence information is hereby incorporated by reference in its entirety), Sus scrofa (NCBI Reference Sequence XP_001928037.1, which sequence information is hereby incorporated by reference in its entirety), Macaca mulatta (NCBI Reference Sequence XP_001111920.1, which sequence information is hereby incorporated by reference in its entirety), and Rattus norvegicus (NCBI Reference Sequence NP_853669.1, which sequence information is hereby incorporated by reference in its entirety).

In one embodiment, the Tbx 3 is administered to the population of pluripotent stem cells in vitro, and the population of pluripotent stem cells administered the Tbx3 are cultured under conditions suitable for differentiation to occur.

Tbx3 is a transcription factor expressed intracellularly. Accordingly, when administering a recombinant form of Tbx3 protein, means of facilitating intracellular delivery should also be employed. Intracellular delivery of proteins can be carried out by a variety of mechanisms, including, but not limited to direct mechanical delivery and carrier-based delivery systems (e.g. covalent protein modification and supramolecular delivery systems) (Fu et al., “Promises and Pitfalls of Intracellular Delivery of Proteins,” Bioconj Chem 25:1602-1608 (2014), which is hereby incorporated by reference in its entirety) including cell penetrating peptide, DNA-assembled recombinant transcription factor (DART), cationic amphiphilic-based delivery reagent, and nanoparticle delivery vehicle.

Mechanical delivery methods, such as microinjection and electroporation deliver the protein directly to the cytosol, and are very useful for in vitro investigations (Fu et al., “Promises and Pitfalls of Intracellular Delivery of Proteins,” Bioconj Chem 25:1602-1608 (2014), which is hereby incorporated by reference in its entirety). Mechanical methods require specialized equipment for physically puncturing cell membranes and thus require direct physical access to the cell. Mechanical methods are low throughput and invasive; therefore, carrier based (i.e. delivery vehicle based) methods are a more attractive mode of delivery.

Covalent protein modification delivery strategies include, but are not limited to, cell-penetrating peptides (CPPs), virus-like particles, supercharged proteins, and nanocarriers (Fu et al., “Promises and Pitfalls of Intracellular Delivery of Proteins,” Bioconj Chem 25:1602-1608 (2014), which is hereby incorporated by reference in its entirety). Cell penetrating peptides are functionalized by modification during or after expression and can be used to introduce a wide range of synthetic and biological components into cells. Several commonly used CPPs which are suitable for intracellular delivery of Tbx3 in accordance with the method described herein, include, without limitation polyarginine peptides, transportant, protamine, maurocalcine, Pep-1, penetratin, HIV-Tat, and M918 (see Stewart et al., “Cell-Penetrating Peptides as Delivery Vehicles for Biology and Medicine,” Organic Biomolecular Chem 6:2242-2255 (2008), which is hereby incorporated by reference in its entirety).

Another suitable intracellular delivery strategy involves fusing Tbx3 to virus-like particles (VLPs) (see e.g., Kaczmarczyk et al., “Protein delivery using engineered virus-like particles,” Proc. Natl. Acad. Sci. U.S.A. 108: 16998-17003 (2011), which is hereby incorporated by reference in its entirety). Alternatively, supercharged proteins, which are a class of engineered or naturally occurring proteins with unusually high positive or negative net theoretical charge, capable of penetrating and delivering macromolecules into mammalian cells, can be used to facilitate intracellular delivery of Tbx3 following the approach described by Thompson et al., “Engineering and identifying supercharged proteins for macromolecule delivery into mammalian cells,” Methods Enzymol. 503: 293-319 (2012), which is hereby incorporated by reference in its entirety). Nanocarriers provide yet another alternative strategy to direct protein delivery, and offer increased options for control of size and surface properties. Nanocarriers may function through covalent attachment between carrier and protein. Covalent bioconjugates may include magnetic nanoparticles, silica nanoparticles, or other nanoparticles (see e.g., Kumar et al., “Chitosan-assisted immobilization of serratiopeptidase on magnetic nanoparticles, characterization and its target delivery,” J. Drug Targeting 22: 123-137 (2014), and Mendez et al., “Delivery of chemically glycosylated cytochrome c immobilized in mesoporous silica nanoparticles induces apoptosis in HeLa cancer cells,” Mol. Pharmacol. 11: 102-111 (2014), which are hereby incorporated by reference in their entirety).

Supramolecular delivery systems are also suitable for delivery of Tbx3 to the population of pluripotent stem cells. Supramolecular delivery systems include, but are not limited to, carrier based delivery systems, liposomes, lipoplexes, polymers, nanoplexes, and nanoparticle-stabilized nanocapsules (Fu et al., “Promises and Pitfalls of Intracellular Delivery of Proteins,” Bioconj Chem 25:1602-1608 (2014), which is hereby incorporated by reference in its entirety). Supramolecular carrier-based delivery systems are modular and operate through reversible association with target proteins. Using noncovalent strategies, proteins and delivery vectors self-assemble, which allows the transport of unmodified proteins into the cell. Suitable supramolecular carrier-based delivery systems include, without limitation, liposomes (Sarker et al., “Intracellular delivery of universal proteins using a lysine headgroup containing cationic liposomes: decipering the uptake mechanism,” Mol. Pharmacol. 11:164-174 (2014) and Furuhata et al., “Intracellular delivery of proteins in complexes with oligoarginine-modified liposomes and the effect of oligoarginine length,” Bioconjugate Chem. 17: 935-942 (2006), which are hereby incorporated by reference in their entirety); lipoplexes, which comprise surfactants, proteins, lipids, polymers, or a combination of these materials, and may be in the format of solid lipid particles, oily suspensions, submicron lipid emulsions, lipid implants, lipid microbubbles, inverse lipid micelles, lipid microtubules, lipospheres, and lipid microcylinders (see e.g., Li et al., “Oral delivery of peptides and proteins using lipid-based drug delivery systems,” Expert Opin. Drug Delivery 9:1289-1304 (2011), which is hereby incorporated by reference in its entirety); and polymers (see e.g., Bhuchar et al., “Degradable thermoresponsive nanogels for protein encapsulation and controlled release,” Bioconjugate Chem. 23:75-83 (2012), and Zhang et al., “pH and reduction dual-bioresponsive polymersomes for efficient intracellular protein delivery,” Langmuir 28: 2056-2065 (2012), which are hereby incorporated by reference in their entirety). Other supramolecular carrier systems that are suitable for delivery of Tbx3 in accordance with the methods described herein include nanoplexes, such as gold nanoparticles (see e.g., Ghosh et al., “Intracellular delivery of a membrane-impermeable enzyme in active form using functionalized gold nanoparticles,” J. Am. Chem. Soc. 132: 2642-2645 (2010), which is hereby incorporated by reference in its entirety), and nanoparticle-stabilized nanocapsules as described in Yang et al., “Drug delivery using nanoparticle-stabilized nanocapsules,” Angew. Chem., Int. Ed. 50: 477-48 (2011), which is hereby incorporated by reference in its entirety).

DNA Assembled Recombinant Transcription Factors (DARTs) can also be used to deliver transcription factors with high efficiency in vivo (Lee et al., “In vivo delivery of transcription factors with multifunctional oligonucleotides,” Nat Matter 14(7): 701-706 (2015), which is hereby incorporated by reference in its entirety). DARTs comprise an oligonucleotide that contains a transcription factor binding sequence and hydrophobic membrane disruptive chains that are masked by acid cleavable galactose residues. The structure of DARTs allows them to disrupt endosomes with minimal toxicity.

In yet another embodiment, intracellular Tbx3 delivery can be achieved using a cationic amphiphilic-based delivery reagent. These delivery reagents are non-peptide based reagents such as the commercially available PULSin™ (Illkirch, France), which allow complex formation with proteins via both electrostatic and hydrophobic interactions, and have been shown to be useful for intracellular protein delivery (Weill et al., “A Practical Approach for Intracellular Protein Delivery,” Cytotechnology 56(1):41-48 (2008), which is hereby incorporated by reference in its entirety). Protein/reagent complexes interact with the cell surface by binding to heparan sulfate proteoglycans. Complexes are internalized by endocytosis, and then the cationic amphiphilic-based reagent induces endosomes escape followed by the complexes disassembly.

In one embodiment of the present invention, a nucleic acid encoding TBX3 or an expression vector comprising a nucleic acid molecule encoding Tbx3 is administered to the population of pluripotent stem cells. Tbx3 is then expressed from the nucleic acid molecule to facilitate Tbx3 protein delivery in the stem cells. Suitable expression vectors include, without limitation, viral vectors, such as retroviral vectors, adeno-associated viral vectors, lentiviral vectors, and herpes viral vectors. In one embodiment, the expression vector is a vector suitable to achieve transient expression of the Tbx3, rather than a vector that is stably integrated into the genome of the pluripotent cell. Transient transfection is particularly suitable in the therapeutic context of the present invention. Expression vectors suitable for transient transfection are known in the art and include, e.g., adenoviral vectors, herpes simplex virus vectors, and vaccinia virus vectors. Other non-genomic integrating expression vectors, such as episomal expression vectors (Yu et al., “Efficient Feeder-free Episomal Reprogramming with Small Molecules,” PLoS One 6(3): e17557 (2011) and Hu et al., “Efficient Generation of Transgene-Free Induced Pluripotent Stem Cells from Normal and Neoplastic Bone Marrow and Cord Blood Mononuclear Cells,” Blood 117:e109-e119 (2011), which are hereby incorporated by reference in their entirety), can also be utilized. Methods of cell transduction to achieve nucleic acid and/or viral vector delivery of Tbx3 are well known in the art, e.g., the use of cationic lipids, calcium phosphate, cationic polymers, DEAE-dextran, magnetic beats, electroporation, and microinjection.

In one embodiment, the differentiation of pluripotent stem cells into neural progenitor cells takes place in vitro. Suitable in vitro culture conditions comprise a suitable substrate, and a nutrient medium to which the differentiation agents are added. Suitable substrates include solid surfaces coated with a positive charge, such as a basic amino acid, exemplified by poly-L-lysine and polyornithine. Substrates can be coated with extracellular matrix components, exemplified by fibronectin. Other permissive extracellular matrixes include Matrigel® (extracellular matrix from Engelbreth-Holm-Swarm tumor cells) and laminin. Also suitable are combination substrates, such as poly-L-lysine combined with fibronectin, laminin, or both.

In one embodiment, Tbx3 is the only differentiation reagent administered to the population of pluripotent stem cells to induce and achieve the differentiation of stem cells to neural progenitor cells. In another embodiment, one or more other differentiation and/or growth factors may be administered to the population of pluripotent stem cells prior to, concurrently with, or subsequent to the administration of Tbx3. Other known neural differentiation agents include growth factors of various kinds, such as epidermal growth factor (EGF), transforming growth factor a (TGF-α), any type of fibroblast growth factor (exemplified by FGF-4, FGF-8, and basic fibroblast growth factor=bFGF), platelet-derived growth factor (PDGF), insulin-like growth factor (IGF-1 and others), high concentrations of insulin, sonic hedgehog, members of the neurotrophin family (such as nerve growth factor=NGF, neurotrophin 3=NT-3, brain-derived neurotrophic factor=BDNF), bone morphogenic proteins (especially BMP-2 & BMP-4), retinoic acid (RA) and ligands to receptors that complex with gp 30 (such as LIF, CNTF, and IL-6). Also suitable are alternative ligands and antibodies that bind to the respective cell-surface receptors for the aforementioned factors. In one embodiment a plurality of differentiation agents is used, which may comprise 2, 3, 4, or more of the agents listed above in combination with Tbx3.

Differentiation factors can be supplied to the cells in a nutrient medium, which is any medium that supports the proliferation or survival of the desired cell type. It is often desirable to use a defined medium that supplies nutrients as free amino acids rather than serum. It is also beneficial to supplement the medium with additives developed for sustained cultures of neural cells. Exemplary are N2 and B27 additives which are commercially available.

Following administration of Tbx3 and culturing of the pluripotent stem to induce neural progenitor cell differentiation, the neural progenitor cells of the preparation may be isolated. Methods of isolating neural progenitor cells from the cultured population of cells can be achieved by selecting for the presence or absence of neural progenitor cells markers. As describe above, neural progenitors can be identified and distinguished based on their expression of particular markers, e.g., CXCR4, Musashi, Nestin, Notch-1, SOX1, SOX2, SSEA-1 and Vimentin. Positive selection for a particular marker or markers can be performed using conventional methods such as immunopanning. The selection methods optionally involve the use of fluorescence sorting (FACS), magnetic sorting (MACS), flow cytometry, or any other methods that allow a rapid, efficient cell sorting. Examples of methods for cell sorting are taught for example in U.S. Pat. No. 6,692,957 to Goldman et al., which is incorporated by reference herein in its entirety, at least for compositions and methods for cell selection and sorting. Alternatively, the neural progenitor preparation can be enriched by negative selection, i.e., selection and removal of contaminating (non-neural progenitor) cell types based on the aforementioned methods using antibodies or other binding reagents that bind to molecular markers expressed by those contaminating cell types. Negative selection can also be effected by incubating the cells successively with a specific antibody, and a preparation of complement that will lyse contaminating cells to which the antibody has bound.

Generally, cell sorting methods use a detectable moiety. Detectable moieties include any suitable direct or indirect label, including, but not limited to, enzymes, fluorophores, biotin, chromophores, radioisotopes, colored beads, electrochemical, chemical-modifying or chemiluminescent moieties. Common fluorescent moieties include fluorescein, cyanine dyes, coumarins, phycoerythrin, phycobiliproteins, dansyl chloride, Texas Red, and lanthanide complexes or derivatives thereof.

Another aspect of the present invention relates to an enriched preparation of neural progenitor cells generated in accordance with the methods described herein. The enriched preparation of neural progenitor has therapeutic utility, and can be utilized in methods of treating various central nervous system injuries and/or disorders. For example, in one embodiment the enriched preparation of neural progenitor cells can be utilized to treat a subject having a spinal cord injury or a traumatic brain injury. In these conditions, the neural progenitor cells are administered to one or more sites within the spinal cord, brain, or eye to facilitate regeneration of injured neurons or other cell types.

In accordance with this and all aspects of the present invention, suitable subjects for treatment with an enriched preparation of progenitor cells include any animal, such as domesticated animals, e.g., cats and dogs; livestock (e.g., cattle, horses, pigs, sheep, and goats); laboratory animals (e.g., mice, rabbits, rats, and guinea pigs); non-human primates, and humans.

Delivery of the cells to the subject can include either a single step or a multiple step injection directly into the nervous system (CNS). Injection is optionally directed into parenchymal or intrathecal sites of the CNS. Such injections can be made unilaterally or bilaterally using precise localization methods such as stereotaxic surgery, optionally with accompanying imaging methods (e.g., high resolution MRI imaging). One of skill in the art recognizes that brain regions vary across species; however, one of skill in the art also recognizes comparable brain regions across species.

The neural progenitor cells are optionally injected as dissociated cells but can also be provided by local placement of non-dissociated cells. In either case, the cellular transplants optionally comprise an acceptable solution. Such acceptable solutions include solutions that avoid undesirable biological activities and contamination. Suitable solutions include an appropriate amount of a pharmaceutically-acceptable salt to render the formulation isotonic. Examples of the pharmaceutically-acceptable solutions include, but are not limited to, saline, Ringer's solution, dextrose solution, and culture media. The pH of the solution is preferably from about 5 to about 8, and more preferably from about 7 to about 7.5.

The injection of the dissociated neural progenitor transplant can be a streaming injection made across the entry path, the exit path, or both the entry and exit paths of the injection device (e.g., a cannula, a needle, or a tube). Automation can be used to provide a uniform entry and exit speed and an injection speed and volume. Optionally a multifocal delivery strategy can be used. Such a multifocal delivery strategy is designed to achieve widespread and dense donor cell engraftment throughout the recipient central nervous system. Injection sites can be chosen to permit contiguous infiltration of migrating donor cells into major brain areas, brainstem, and spinal white matter tracts, without hindrance (or with limited hindrance) from intervening gray matter structures.

The number of neural progenitor cells transplanted can range from about 10²-10⁸ at each transplantation (e.g., injection site), depending on the size and species of the recipient, and the volume of tissue requiring regeneration or replacement. Single transplantation (e.g., injection) doses can span ranges of 10³-10⁵, 10⁴-10⁷, and 10⁵-10⁸ cells, or any amount in total for a transplant recipient patient.

Since the CNS is an immunologically privileged site, transplanted cells, including xenogeneic, can survive and, optionally, no immunosuppressant drugs are administered in conjunction with the treatment. Alternatively, a typical regimen of immunosuppressant agents are administered in conjunction with the treatment methods described herein. Immunosuppressant agents and their dosing regimens are known to one of skill in the art and include such agents as Azathioprine, Azathioprine Sodium, Cyclosporine, Daltroban, Gusperimus Trihydrochloride, Sirolimus, and Tacrolimus. Dosages ranges and duration of the regimen can be varied with the disorder being treated; the extent of rejection; the activity of the specific immunosuppressant employed; the age, body weight, general health, sex and diet of the subject; the time of administration; the route of administration; the rate of excretion of the specific immunosuppressant employed; the duration and frequency of the treatment; and drugs used in combination. One of skill in the art can determine acceptable dosages for and duration of immunosuppression therapy. The dosage regimen can be adjusted by the individual physician in the event of any contraindications or change in the subject's status.

In some embodiments it is desirable to induce further differentiation of the produced neural progenitor cells to produce, for example, a preparation of neuronal progenitor cells, glial progenitor cells, or retinal progenitor cells. This can be achieved by contacting the enriched preparation of neural progenitor cells produced during or after culturing with one or more reagents suitable to induce differentiation and production of the desired cells type (e.g., retinal progenitor cells, neuronal progenitor cells, glial progenitor cells).

In accordance with this embodiment, the neural progenitor cell preparation can be contacted with the one or more reagents in conjunction with the Tbx3 or following Tbx3 administration. In some embodiments, it is desirable to co-administer the Tbx3 and the one or more additional differentiation reagents for the duration of time necessary to produce the desired preparation of neural progenitors, then cease administration of Tbx3 while continuing administration of the other reagent(s) to induce further differentiation.

In one embodiment, the desired cell type is midbrain progenitor cells. To produce midbrain progenitor cells from the enriched preparation of neural progenitor cells, the preparation of neural progenitor cells is contacted with an active form of the protein, Emx2, or nucleic acid molecule encoding such protein (Empty spiracles homeobox 2; UnitProt identifier No. Q04743, which sequence information is hereby incorporated by reference in its entirety).

In another embodiment, the desired cell type is hindbrain progenitor cells. To produce hindbrain progenitor cells from the enriched preparation of neural progenitor cells, the preparation of neural progenitor cells is contacted with an active form of the protein, Irx2, or nucleic acid molecule encoding such protein (Iroquois homeobox 2; UnitProt identifier No. Q9BZI1, which sequence information is hereby incorporated by reference in its entirety).

In another embodiment, the desired cell type is retinal progenitor cells. To produce retinal progenitor cells from the enriched preparation of neural progenitor cells, the preparation of neural progenitor cells is contacted with an active form of the protein Pax6, or a nucleic acid molecule encoding such protein.

The pax6 gene encodes a homeobox and paired domain-containing protein that binds DNA and functions as a regulator of transcription. Activity of this protein is key to the development of neural tissues, particularly neural tissue of the eye. This gene is regulated by multiple enhancers located hundreds of kilobases from this locus. The use of alternative promoters and alternative splicing result in multiple transcript variants encoding different isoforms.

In humans, Pax6 (isoform 1) is encoded by the nucleotide sequence of SEQ ID NO: 6 (NCBI Reference Sequence NP_0057653.3; UniProt identifier P26367-1):

| SEQ ID NO: 6 | |
|---|---|
| Pax6 | |
| aatattttgt gtgagagcga gcggtgcatt tgcatgttgc ggagtgatta gtgggtttga | 60 |
| aaagggaacc gtggctcggc ctcatttccc gctctggttc aggcgcagga ggaagtgttt | 120 |
| tgctggagga tgatgacaga ggtcaggctt cgctaatggg ccagtgagga gcggtggagg | 180 |
| cgaggccggg cgccggcaca cacacattaa cacacttgag ccatcaccaa tcagcatagg | 240 |
| aatctgagaa ttgctctcac acaccaaccc agcaacatcc gtggagaaaa ctctcaccag | 300 |
| caactccttt aaaacaccgt catttcaaac cattgtggtc ttcaagcaac aacagcagca | 360 |
| caaaaaaccc caaccaaaca aaactcttga cagaagctgt gacaaccaga aaggatgcct | 420 |
| cataaagggg gaagacttta actaggggcg cgcagatgtg tgaggccttt tattgtgaga | 480 |
| gtggacagac atccgagatt tcagagcccc atattcgagc cccgtggaat cccgcggccc | 540 |
| ccagccagag ccagcatgca gaacagtcac agcggagtga atcagctcgg tggtgtcttt | 600 |
| gtcaacgggc ggccactgcc ggactccacc cggcagaaga ttgtagagct agctcacagc | 660 |
| ggggcccggc cgtgcgacat ttcccgaatt ctgcaggtgt ccaacggatg tgtgagtaaa | 720 |
| attctgggca ggtattacga gactggctcc atcagaccca gggcaatcgg tggtagtaaa | 780 |
| ccgagagtag cgactccaga agttgtaagc aaaatagccc agtataagcg ggagtgcccg | 840 |
| tccatctttg cttgggaaat ccgagacaga ttactgtccg agggggtctg taccaacgat | 900 |
| aacataccaa gcgtgtcatc aataaacaga gttcttcgca acctggctag cgaaaagcaa | 960 |
| cagatgggcg cagacggcat gtatgataaa ctaaggatgt tgaacgggca gaccggaagc | 1020 |
| tggggcaccc gccctggttg gtatccgggg acttcggtgc cagggcaacc tacgcaagat | 1080 |
| ggctgccagc aacaggaagg agggggagag aataccaact ccatcagttc caacggagaa | 1140 |
| gattcagatg aggctcaaat gcgacttcag ctgaagcgga agctgcaaag aaatagaaca | 1200 |
| tcctttaccc aagagcaaat tgaggccctg gagaaagagt ttgagagaac ccattatcca | 1260 |
| gatgtgtttg cccgagaaag actagcagcc aaaatagatc tacctgaagc aagaatacag | 1320 |
| gtatggtttt ctaatcgaag ggccaaatgg agaagagaag aaaaactgag gaatcagaga | 1380 |
| agacaggcca gcaacacacc tagtcatatt cctatcagca gtagtttcag caccagtgtc | 1440 |
| taccaaccaa ttccacaacc caccacaccg gtttcctcct tcacatctgg ctccatgttg | 1500 |
| ggccgaacag acacagccct cacaaacacc tacagcgctc tgccgcctat gcccagcttc | 1560 |
| accatggcaa ataacctgcc tatgcaaccc ccagtcccca gccagacctc ctcatactcc | 1620 |
| tgcatgctgc ccaccagccc ttcggtgaat gggcggagtt atgataccta caccccccca | 1680 |
| catatgcaga cacacatgaa cagtcagcca atgggcacct cgggcaccac ttcaacagga | 1740 |
| ctcatttccc ctggtgtgtc agttccagtt caagttcccg gaagtgaacc tgatatgtct | 1800 |
| caatactggc caagattaca gtaaaaaaaa aaaaaaaaaa aaaaaggaaa ggaaatattg | 1860 |
| tgttaattca gtcagtgact atggggacac aacagttgag ctttcaggaa agaaagaaaa | 1920 |
| atggctgtta gagccgcttc agttctacaa ttgtgtcctg tattgtacca ctggggaagg | 1980 |
| aatggacttg aaacaaggac ctttgtatac agaaggcacg atatcagttg gaacaaatct | 2040 |
| tcattttggt atccaaactt ttattcattt tggtgtatta tttgtaaatg ggcatttgta | 2100 |
| tgttataatg aaaaaaagaa caatgtagac tggatggatg tttgatctgt gttggtcatg | 2160 |
| aagttgtttt tttttttttt aaaaagaaaa ccatgatcaa caagctttgc cacgaattta | 2220 |
| agagttttat caagatatat cgaatacttc tacccatctg ttcatagttt atggactgat | 2280 |
| gttccaagtt tgtatcattc ctttgcatat aattaaacct ggaacaacat gcactagatt | 2340 |
| tatgtcagaa atatctgttg gttttccaaa ggttgttaac agatgaagtt tatgtgcaaa | 2400 |
| aaagggtaag atataaattc aaggaagaaa aaaagttgat agctaaaagg tagagtgtgt | 2460 |
| cttcgatata atccaatttg ttttatgtca aaatgtaagt atttgtcttc cctagaaatc | 2520 |
| ctcagaatga tttctataat aaagttaatt tcatttatat ttgacaagaa tatagatgtt | 2580 |
| ttatacacat tttcatgcaa tcatacgttt cttttttggc cagcaaaagt taattgttct | 2640 |
| tagatatagt tgtattactg ttcacggtcc aatcattttg tgcatctaga gttcattcct | 2700 |
| aatcaattaa aagtgcttgc aagagtttta aacttaagtg ttttgaagtt gttcacaact | 2760 |
| acatatcaaa attaaccatt gttgattgta aaaaaccatg ccaaagcctt tgtatttcct | 2820 |
| ttattataca gttttctttt taaccttata gtgtggtgtt acaaatttta tttccatgtt | 2880 |
| agatcaacat tctaaaccaa tggttacttt cacacacact ctgttttaca tcctgatgat | 2940 |
| ccttaaaaaa taatccttat agataccata aatcaaaaac gtgttagaaa aaaattccac | 3000 |
| ttacagcagg gtgtagatct gtgcccattt atacccacaa catatataca aaatggtaac | 3060 |
| atttcccagt tagccattta attctaaagc tcaaagtcta gaaataattt aaaaatgcaa | 3120 |
| caagcgatta gctaggaatt gttttttgaa ttaggactgg cattttcaat ctgggcagat | 3180 |
| ttccattgtc agcctatttc aacaatgatt tcactgaagt atattcaaaa gtagatttct | 3240 |
| taaaggagac tttctgaaag ctgttgcctt tttcaaatag gccctctccc ttttctgtct | 3300 |
| ccctcccctt tgcacaagag gcatcatttc ccattgaacc actacagctg ttcccatttg | 3360 |
| aatcttgctt tctgtgcggt tgtggatggt tggagggtgg aggggggatg ttgcatgtca | 3420 |
| aggaataatg agcacagaca catcaacaga caacaacaaa gcagactgtg actggccggt | 3480 |
| gggaattaaa ggccttcagt cattggcagc ttaagccaaa cattcccaaa tctatgaagc | 3540 |
| agggcccatt gttggtcagt tgttatttgc aatgaagcac agttctgatc atgtttaaag | 3600 |
| tggaggcacg cagggcagga gtgcttgagc ccaagcaaag gatggaaaaa aataagcctt | 3660 |
| tgttgggtaa aaaaggactg tctgagactt tcatttgttc tgtgcaacat ataagtcaat | 3720 |
| acagataagt cttcctctgc aaacttcact aaaaagcctg ggggttctgg cagtctagat | 3780 |
| taaaatgctt gcacatgcag aaacctctgg ggacaaagac acacttccac tgaattatac | 3840 |
| tctgctttaa aaaaatcccc aaaagcaaat gatcagaaat gtagaaatta atggaaggat | 3900 |
| ttaaacatga ccttctcgtt caatatctac tgttttttag ttaaggaatt acttgtgaac | 3960 |
| agataattga gattcattgc tccggcatga aatatactaa taattttatt ccaccagagt | 4020 |
| tgctgcacat ttggagacac cttcctaagt tgcagttttt gtatgtgtgc atgtagtttt | 4080 |
| gttcagtgtc agcctgcact gcacagcagc acatttctgc aggggagtga gcacacatac | 4140 |
| gcactgttgg tacaattgcc ggtgcagaca tttctacctc ctgacatttt gcagcctaca | 4200 |
| ttccctgagg gctgtgtgct gagggaactg tcagagaagg gctatgtggg agtgcatgcc | 4260 |
| acagctgctg gctggcttac ttcttccttc tcgctggctg taatttccac cacggtcagg | 4320 |
| cagccagttc cggcccacgg ttctgttgtg tagacagcag agactttgga gacccggatg | 4380 |
| tcgcacgcca ggtgcaagag gtgggaatgg gagaaaagga gtgacgtggg agcggagggt | 4440 |
| ctgtatgtgt gcacttgggc acgtatatgt gtgctctgaa ggtcaggatt gccagggcaa | 4500 |
| agtagcacag tctggtatag tctgaagaag cggctgctca gctgcagaag ccctctggtc | 4560 |
| cggcaggatg ggaacggctg ccttgccttc tgcccacacc ctagggacat gagctgtcct | 4620 |
| tccaaacaga gctccaggca ctctcttggg gacagcatgg caggctctgt gtggtagcag | 4680 |
| tgcctgggag ttggcctttt actcattgtt gaaataattt ttgtttatta tttatttaac | 4740 |
| gatacatata tttatatatt tatcaatggg gtatctgcag ggatgttttg acaccatctt | 4800 |
| ccaggatgga gattatttgt gaagacttca gtagaatccc aggactaaac gtctaaattt | 4860 |
| tttctccaaa cttgactgac ttgggaaaac caggtgaata gaataagagc tgaatgtttt | 4920 |
| aagtaataaa cgttcaaact gctctaagta aaaaaatgca ttttactgca atgaatttct | 4980 |
| agaatatttt tcccccaaag ctatgcctcc taacccttaa atggtgaaca actggtttct | 5040 |
| tgctacagct cactgccatt tcttcttact atcatcacta ggtttcctaa gattcactca | 5100 |
| tacagtatta tttgaagatt cagctttgtt ctgtgaatgt catcttagga ttgtgtctat | 5160 |
| attcttttgc ttatttcttt ttactctggg cctctcatac tagtaagatt ttaaaaagcc | 5220 |
| ttttcttctc tgtatgtttg gctcaccaag gcgaaatata tattcttctc tttttcattt | 5280 |
| ctcaagaata aacctcatct gcttttttgt ttttctgtgt tttggcttgg tactgaatga | 5340 |
| ctcaactgct cggttttaaa gttcaaagtg taagtactta gggttagtac tgcttatttc | 5400 |
| aataatgttg acggtgacta tctttggaaa gcagtaacat gctgtcttag aaatgacatt | 5460 |
| aataatgggc ttaaacaaat gaataggggg gtccccccac tctccttttg tatgcctatg | 5520 |
| tgtgtctgat ttgttaaaag atggacaggg aattgattgc agagtgtcgc ttccttctaa | 5580 |
| agtagtttta ttttgtctac tgttagtatt taaagatcct ggaggtggac ataaggaata | 5640 |
| aatggaagag aaaagtagat attgtatggt ggctactaaa aggaaattca aaaagtctta | 5700 |
| gaacccgagc acctgagcaa actgcagtag tcaaaatatt tatctcatgt taaagaaagg | 5760 |
| caaatctagt gtaagaaatg agtaccatat agggttttga agttcatata ctagaaacac | 5820 |
| ttaaaagata tcatttcaga tattacgttt ggcattgttc ttaagtattt atatctttga | 5880 |
| gtcaagctga taattaaaaa aaatctgtta atggagtgta tatttcataa tgtatcaaaa | 5940 |
| tggtgtctat acctaaggta gcattattga agagagatat gtttatgtag taagttatta | 6000 |
| acataatgag taacaaataa tgtttccaga agaaaggaaa acacattttc agagtgcgtt | 6060 |
| tttatcagag gaagacaaaa atacacaccc ctctccagta gcttattttt acaaagccgg | 6120 |
| cccagtgaat tagaaaaaca aagcacttgg atatgatttt tggaaagccc aggtacactt | 6180 |
| attattcaaa atgcactttt actgagtttg aaaagtttct tttatattta aaataagggt | 6240 |
| tcaaatatgc atattcaatt tttatagtag ttatctattt gcaaagcata tattaactag | 6300 |
| taattggctg ttaattttat agacatggta gccagggaag tatatcaatg acctattaag | 6360 |
| tattttgaca agcaatttac atatctgatg acctcgtatc tctttttcag caagtcaaat | 6420 |
| gctatgtaat tgttccattg tgtgttgtat aaaatgaatc aacacggtaa gaaaaaggtt | 6480 |
| agagttatta aaataataaa ctgactaaaa tactcatttg aatttattca gaatgttcat | 6540 |
| aatgctttca aaggacatag cagagctttt gtggagtatc cgcacaacat tatttattat | 6600 |
| ctatggacta aatcaatttt ttgaagttgc tttaaaattt aaaagcacct ttgcttaata | 6660 |
| taaagccctt taattttaac tgacagatca attctgaaac tttattttga aaagaaaatg | 6720 |
| gggaagaatc tgtgtcttta gaattaaaag aaatgaaaaa aataaacccg acattctaaa | 6780 |
| aaaatagaat aagaaacctg atttttagta ctaatgaaat agcgggtgac aaaatagttg | 6840 |
| tctttttgat tttgatcaca aaaaataaac tggtagtgac aggatatgat ggagagattt | 6900 |
| gacatcctgg caaatcactg tcattgattc aattattcta attctgaata aaagctgtat | 6960 |
| acagtaaaa | 6969 |

Alternative splicing generates three isoforms of Pax6. The amino acid sequence of Pax (isoform 1) (UniProt identifier P26367-1), which has been designated the ‘canonical’ sequence is provided below as SEQ ID NO: 7 below.

```
SEQ ID NO: 7
Met Gln Asn Ser His Ser Gly Val Asn Gln Leu Gly Gly Val Phe Val
1 5 10 15
Asn Gly Arg Pro Leu Pro Asp Ser Thr Arg Gln Lys Ile Val Glu Leu
                        20 25 30
Ala His Ser Gly Ala Arg Pro Cys Asp Ile Ser Arg Ile Leu Gln Val
                35 40 45
Ser Asn Gly Cys Val Ser Lys Ile Leu Gly Arg Tyr Tyr Glu Thr Gly
        50 55 60
Ser Ile Arg Pro Arg Ala Ile Gly Gly Ser Lys Pro Arg Val Ala Thr
65 70 75 80
Pro Glu Val Val Ser Lys Ile Ala Gln Tyr Lys Arg Glu Cys Pro Ser
                                85 90 95
Ile Phe Ala Trp Glu Ile Arg Asp Arg Leu Leu Ser Glu Gly Val Cys
                        100 105 110
Thr Asn Asp Asn Ile Pro Ser Val Ser Ser Ile Asn Arg Val Leu Arg
                115 120 125
Asn Leu Ala Ser Glu Lys Gln Gln Met Gly Ala Asp Gly Met Tyr Asp
        130 135 140
Lys Leu Arg Met Leu Asn Gly Gln Thr Gly Ser Trp Gly Thr Arg Pro
145 150 155 160
Gly Trp Tyr Pro Gly Thr Ser Val Pro Gly Gln Pro Thr Gln Asp Gly
                                165 170 175
Cys Gln Gln Gln Glu Gly Gly Gly Glu Asn Thr Asn Ser Ile Ser Ser
                        180 185 190
Asn Gly Glu Asp Ser Asp Glu Ala Gln Met Arg Leu Gln Leu Lys Arg
                195 200 205
Lys Leu Gln Arg Asn Arg Thr Ser Phe Thr Gln Glu Gln Ile Glu Ala
        210 215 220
Leu Glu Lys Glu Phe Glu Arg Thr His Tyr Pro Asp Val Phe Ala Arg
225 230 235 240
Glu Arg Leu Ala Ala Lys Ile Asp Leu Pro Glu Ala Arg Ile Gln Val
                                245 250 255
Trp Phe Ser Asn Arg Arg Ala Lys Trp Arg Arg Glu Glu Lys Leu Arg
                        260 265 270
Asn Gln Arg Arg Gln Ala Ser Asn Thr Pro Ser His Ile Pro Ile Ser
                275 280 285
Ser Ser Phe Ser Thr Ser Val Tyr Gln Pro Ile Pro Gln Pro Thr Thr
        290 295 300
Pro Val Ser Ser Phe Thr Ser Gly Ser Met Leu Gly Arg Thr Asp Thr
305 310 315 320
Ala Leu Thr Asn Thr Tyr Ser Ala Leu Pro Pro Met Pro Ser Phe Thr
                                325 330 335
Met Ala Asn Asn Leu Pro Met Gln Pro Pro Val Pro Ser Gln Thr Ser
                        340 345 350
Ser Tyr Ser Cys Met Leu Pro Thr Ser Pro Ser Val Asn Gly Arg Ser
                355 360 365
Tyr Asp Thr Tyr Thr Pro Pro His Met Gln Thr His Met Asn Ser Gln
        370 375 380
Pro Met Gly Thr Ser Gly Thr Thr Ser Thr Gly Leu Ile Ser Pro Gly
385 390 395 400
Val Ser Val Pro Val Gln Val Pro Gly Ser Glu Pro Asp Met Ser Gln
                                405 410 415
Tyr Trp Pro Arg Leu Gln
                        420
```

Mammalian homologs of Pax6 have been found in Bos taurus (NCBI Reference Sequence NP_001035735.1, which sequence information is hereby incorporated by reference in its entirety), Mus musculus (NCBI Reference Sequence NP_001231130.1, which sequence information is hereby incorporated by reference in its entirety), Rattus norvegicus (NCBI Reference Sequence NP_037133.1, which sequence information is hereby incorporated by reference in its entirety), Sus scrofa (NCBI Reference Sequence NP_001231101.1, which sequence information is hereby incorporated by reference in its entirety), Canis lupus familiaris (NCBI Reference Sequence NP_001091013.1, which sequence information is hereby incorporated by reference in its entirety), Ovis aries (NCBI Reference Sequence NP_001171523.1, which sequence information is hereby incorporated by reference in its entirety), Oryctolagus cuniculus (NCBI Reference Sequence NP_001075686.1, which sequence information is hereby incorporated by reference in its entirety), Papio anubis (NCBI Reference Sequence NP_001162400.1, which sequence information is hereby incorporated by reference in its entirety), Monodelphis domestica (NCBI Reference Sequence XP_001368528.2, which sequence information is hereby incorporated by reference in its entirety), Pan troglodytes (NCBI Reference Sequence XP_003312778.1, which sequence information is hereby incorporated by reference in its entirety), and Macaca mulatta (NCBI Reference Sequence NP_001253186.1, which sequence information is hereby incorporated by reference in its entirety).

Accordingly, another aspect of the present invention is directed to a method of producing an enriched preparation of retinal progenitor cells from a population of stem cells. This method involves administering Tbx3 and Pax6 to the population of stem cells and culturing the population of stem cells, to which Tbx3 and Pax6 have been administered, under conditions suitable to produce the enriched preparation of retinal progenitor cells from the population of stem cells. In one embodiment, Tbx3 and Pax6 are the only reagents administered to the population of stem cells that active in inducing retinal progenitor cell production. A related aspect of the present invention is directed to an enriched preparation of retinal progenitor cells produced in accordance with this method.

Retinal progenitor cells are multipotent cells capable of giving rise to the retinal pigmented epithelium and all neurons, photoreceptors, and the Muller glia of the eye. These progenitor cells have a simple bipolar morphology, and in most cases undergo their mitotic divisions at the ventricular surface. Immediately after their final mitotic division, one or both of the daughter cells begin to express characteristics of differentiating neurons. In the early embryonic retina, many of the divisions of the progenitor cells are symmetric, where both progeny of a particular division can remain progenitor cells and continue to divide. However, some of the mitotic divisions are asymmetric, with a particular division yielding a neuron and another progenitor cell, or two neurons of different types.

Retinal progenitor cells may be functionally characterized according to their ability to give rise to multiple lineages, or may be characterized according to the expression of genes associated with retinal development. In particular, the differentiation of retinal progenitors from stem cells is characterized by the acquisition of the expression of one or more, two or more, or three or more eye field transcription factors. These eye field transcription factors include Tbx3, Rx, c-myb, Crx, Pax6, Six3, Lhx2, til, Optx2, and the like. The sequences of these genes and reagents for detecting their expression are known in the art and readily obtainable. Antibodies specific for the protein products are well known and available in the art.

Retinal progenitor cells express one or more eye field transcription factors at a level of at least about 10 fold more than the expression level observed in stem cells, and may be increased to at least about 100 fold or more relative to the expression level found in stem cells.

An enriched preparation of retinal progenitor cells, as referred to herein, is a preparation or population of cells comprising at least about 60% retinal progenitor cells, at least about 70% retinal progenitor cells, 75% retinal progenitor cells, 80% retinal progenitor cells, of more, for example, about 85%, 90%, 95%, 96%, 97%, 98%, 99%, 100% retinal progenitor cells.

The enriched preparation of retinal progenitor cells as described herein is relatively devoid, e.g., containing less than 40, 30, 25, 20, 15, 10, 9, 8, 7, 6, 5, 4, 3, 2, or 1% of other cell types such as pluripotent stem cells, neuronal progenitors, glial progenitors, astrocytes, oligodendrocytes, etc. Methods of identifying the presence of contaminating cell types is described supra.

Methods of selecting for an isolating retinal progenitor cells to further purify and enhance the retinal progenitor cell population are described supra. These methods employ retinal progenitor cell selection based on the expression of the retinal progenitor cell specific markers described supra, i.e., the eye field transcription factors of Tbx3, Rx, c-myb, Crx, Pax6, Six3, Lhx2, til, Optx2, and the like.

In one embodiment, the preparation of retinal progenitor cells are produced from a population of pluripotent stem cells. Suitable populations of pluripotent stem cells, e.g., embryonic stem cells, fetal stem cells, and iPSCs are described supra. In another embodiment the preparation of retinal progenitor cells is produced from a population of neural progenitor cells.

Administration of Tbx3 and Pax6 is carried out as described supra for Tbx3 alone. In one embodiment, recombinant Tbx3 and Pax6 proteins are delivered intracellularly using any of the intracellular delivery vehicles described supra. Alternatively, one or more nucleic acid molecules encoding Tbx3 and Pax6 or expression vectors comprising nucleic acid molecules encoding Tbx3 and Pax6 can be administered to the population of stem cells, and Tbx3 and Pax6 are expressed by the one or more expression vectors during culture. Suitable expression vectors are described supra. In one embodiment, nucleic acid molecules or expression vectors expressing Tbx3 and Pax6 are transiently (not stably) expressed in the population of pluripotent cells. Methods of achieving transient transfection and transient expression are known in the art.

In one embodiment, the Tbx3 and Pax6 are administered simultaneously to the population of stem cells. In another embodiment, Tbx3 and Pax6 are administered sequentially, where Tbx3 is administered first for a duration of time sufficient to induce neural progenitor cells differentiation in the population, and Pax6 is administered subsequent to Tbx3 withdrawal to induce retinal progenitor cell differentiation in the population of neural progenitor cells. In another embodiment, Tbx3 and Pax6 administration is carried out sequentially, but with a period of co-administration.

In one embodiment retinal progenitor cell production is carried out in vitro using retinal differentiation culture conditions. Such culture conditions include a suitable medium, for example Dulbecco's minimum essential medium, and the like, and may comprise knock-out serum; serum replacement; etc. at a suitable concentration, e.g. at about 10%; and comprising B-27 supplement. In one embodiment, one or more other retinal differentiating agents may be administered in conjunction with the Tbx3 and Pax6. Suitable retinal differentiating agents include, without limitation, an antagonist of bone morphogenetic protein (BMP) signaling pathways; an antagonist of wnt signaling pathways; an IGF1R ligand; and a molecule that provides FGF2 activity. The cells are cultured in the presence of the differentiating agents for a period of time sufficient to allow retinal differentiation. Retinal differentiation may be accomplished in at least about 1 week, at least about 2 weeks, at least about 3 weeks or more, and usually not more than about 6 weeks.

In one aspect of the invention, the retinal progenitor cells are cultured under conditions suitable to form retinal organoids. Retinal organoids are complex, three-dimensional cellular structures resembling the in vivo retina tissue architecture that are formed in culture. Methods and culture conditions suitable for producing retinoid organoids in culture from retinal progenitor cells are known in the art, see e.g., Volkner et al., “Retinal Organoids from Pluripotent Stem Cells Efficiently Recapitulate Retinogenesis,” Stem Cell Reports 6(4):525-538 (2016), which is hereby incorporated by reference in its entirety. However, the combined administration of Tbx3 and Pax6 to the retinal progenitor cell preparation to produce these retinoid organoids has not previously been described, and is expected to enhance retinal organoid formation. Retinal organoids produced via the methods described herein are useful research and drug screening tools.

Another aspect of the present invention relates to a method of treating a retinal disorder using a preparation of neural progenitor cells or retinal progenitor cells produced via the methods described herein. The method involves selecting a subject having a retinal disorder, and administering, to the subject, the enriched preparation of retinal or neural progenitor cells produced in accordance with the methods of the present invention.

The phrase “retinal disorder” is used to describe a defect in the tissue of the retina. “Retinal tissue” refers to the neural cells and associated vasculature that line the back of the eye. Structures within retinal tissue include the macula and fovea. Retinal tissue further includes the tissue that is juxtaposed to these neural cells (e.g. pigment epithelia) and associated vasculature. Retinal disorders may result from infection, injury, or a degenerative condition. Degenerative conditions include, but are not limited to, age-related macular degeneration, retinitis pigmentosa, diabetic retinopathy, cone-rod dystrophies, glaucoma and limbal epithelial cell deficiency. A retinal disorder may also be caused by physical damage. The term retinal disorder includes also any condition that leads to the impairment of the retina's normal function. Treating a retinal disorder as described herein refers to ameliorating the effects of, or delaying, halting or reversing the progress of, or delaying or preventing the onset of the disorder.

Neural progenitor cells or retinal progenitor cells can be administered to a subject having a retinal disorder using methods known in the art and suitable for facilitating the delivery of cells to treat the retinal tissue disorder. The cells may be directly administered to one or more sites within the eye of the patient through a variety of modes including, but not limited to, retrobulbar injection, intravitreous injection, and subretinal injection.

Another aspect of the present invention is directed to a kit containing a collection of reagents suitable for neural progenitor cell and/or retinal progenitor cell production. In one embodiment, the kit comprises recombinant Tbx3 and a suitable intracellular delivery vehicle. In another embodiment, the kit comprises an expression vector comprising a nucleic acid molecule encoding Tbx3. In one embodiment, the expression vector comprising the nucleic acid molecule encoding Tbx3 is suitable for transient, but not stable transfection of target cells. For retinal progenitor cell production, the kit further comprises recombinant Pax6 and a suitable intracellular delivery vehicle or an expression vector comprising a nucleic acid molecule encoding Pax6. The kit may further comprise culture medium, culture dishes, and/or other growth factors and/or differentiation factors suitable for promoting neural and/or retinal progenitor cell differentiation as described supra.

### EXAMPLES

### Material and Methods for Examples 1-9

Animals.

Xenopus laevis embryos were obtained either by in vitro fertilization or natural mating and staged according to Nieuwkoop et al., “Normal Table of Xenopus Laevis (Daudin): A Systematical & Chronological Survey of the Development from the Fertilized Egg till the End of the Fertilized Egg Till the End of Metamorp,” (1994), which is hereby incorporated by reference in its entirety. All procedures were approved by the SUNY UMU Committee for the Humane Use of Animals.

Plasmid Construction.

The Tbx3MO target sequences located in the 5′UTR of tbx3.L and tbx3.S were PCR amplified (see Table 1 below for a listing of all primer sequences) from X. laevis genomic DNA (gDNA) and cloned in frame with vYFP to generate pCS2R.Tbx3.L-vYFP and pCS2R.Tbx3.SvYFP. To generate pCS2R.X1Tbx3GR, GR was PCR amplified from pCS2+Tbx5-EnRGR (Horb et al., “Tbx5 is Essential for Heart Development,” Development 126:1739-1751 (1999), which is hereby incorporated by reference in its entirety) and inserted in frame with XlTbx3. The tbx3.L DNA binding domain from gDNA and the EnR-GR domain from pCS2+Tbx5-EnR-GR (Horb et al., “Tbx5 is Essential for Heart Development,” Development 126:1739-1751 (1999), which is hereby incorporated by reference in its entirety) were PCR amplified and cloned into pCS2R to create pCS2R.Tbx3LDBD-EnR-GR. RN3P-VP16-DBD-GR (Takabatake et al., “Conserved Expression Control and Shared Activity Between Cognate T-box Genes Tbx2 and Tbx3 in Connection with Sonic Hedgehog Signaling During Xenopus Eye Development,” Dev Growth Differ 44:257-271 (2002), which is hereby incorporated by reference in its entirety).

Microinjection and Tissue Transplants.

Morpholinos (MOs) were obtained from Gene Tools LLC (Philomath, Oreg.). Capped RNA was synthesized from NotI linearized plasmids using the SP6 mMessage Machine Kit (Ambion, Austin, Tex.). See figure legends for developmental stage and amount of RNA/morpholino injected. Stage 9 animal caps were removed and cultured to stage 15. For in situ hybridization, stage 15 caps were transferred to 0.1×MMR (with or without dexamethasone) and fixed when sibling control embryos reached stage 22 (Kolm et al., “Efficient Hormone-Inducible Protein Function in Xenopus Laevis,” Dev Biol 171:267-272 (1995), which is hereby incorporated by reference in its entirety). Animal Cap Transplant (ACT) was performed at stage 15 as previously described (Viczian et al., “Tissue Determination using the Animal Cap Transplant (ACT) Assay in Xenopus Laevis,” J Vis Exp 39:1932 (2010), which is hereby incorporated by reference in its entirety). For eye field to eye field transplant assays, the dorsal animal blastomeres (D1) of 8-cell staged embryos was unilaterally injected with YFP and the gene(s) of interest. At stage 15, the central region of the YFP-positive eye field (˜⅓ of the total eye field area) was surgically removed from donor embryos using sharp forceps and grafted to the host eye field after removal of a similarly sized region from the host.

In Situ Hybridization. In situ hybridization was carried out as previously described (Zuber et al., “Specification of the Vertebrate Eye by a Network of Eye Field Transcription Factors,” Development 130:5155-5167 (2003), which is hereby incorporated by reference in its entirety). Digoxigenin (DIG)-labelled antisense RNA probes were generated from pCS2R.Tbx3, pBSSKII.Bmp4, pGEMTEZ.Rax, pCS2R.Pax6, pCS2.Otx2, pBSSKII.Xag1, and pCS2+.X1FoxG1 using RNAPolymerase Plus (Ambion, Austin, Tex.).

Reverse Transcription PCR.

Total RNA was extracted from animal caps (10 per condition) or dissected tissue (20 per condition) or whole embryos (5 per condition) using RNAzol RT (Molecular Research Center, Inc., Cincinnati, Ohio) and cDNA synthesized using 1 μg total RNA (MMLV Reverse Transcriptase; Promega, Madison, Wis.). PCR primer information is in Table 1 below.

Western Blotting.

Sample preparation performed as previously described using 30 μg total protein per sample (Wong et al., “Efficient Retina Formation Requires Suppression of Both Activin and BMP Signaling Pathways in Pluripotent Cells,” Biol Open 4:573-583 (2015), which is hereby incorporated by reference in its entirety). Antibodies were anti-GFP antibody (1:1000; ThermoFisher), polyclonal anti-β-actin (1:1000; Cell Signaling, Danvers, Mass.), and goat anti-rabbit HRP-conjugated antibody (1:2000; Millipore, Billerica, Mass.).

Immunostaining and Imaging.

Sections were stained as previously described (Viczian et al., “XOtx5b and XOtx2 Regulate Photoreceptor and Bipolar Fates in the Xenopus Retina,” Development 130:1281-1294 (2003); Martinez-De Luna et al., “Maturin is a Novel Protein Required for Differentiation During Primary Neurogenesis,” Dev Biol 384:26-40 (2013), which are hereby incorporated by reference in their entirety), except blocking solution for Islet1/2 and Sox2 antibodies contained 0.5% PBST, 10% HIGS, 1% BSA, and 1% Saponin. Primary antibodies: mouse anti-XAP-2 monoclonal (1:25, clone 5B9, DHSB, Iowa City, Iowa), rabbit anti-GFP polyclonal (1:1000; Invitrogen, Grand Island, N.Y.), mouse anti-class II-β tubulin (1:1000; clone 7B9, MMS-422P, BioLegend, San Diego, Calif.), mouse anti-Islet-1/2 (1:100; clone 39.4D5; DSHB, Iowa City, Iowa), rabbit anti-Gαt1 polyclonal (1:100; Sc-389, Santa Cruz Biotechnology, Dallas, Tex.), and rabbit anti-Sox2 polyclonal (1:500, ab97959, Abcam, Cambridge, Mass.). Secondary antibodies: donkey anti-rabbit IgG Alexa 488 (1:1000), goat anti-mouse IgG Alexa 555 (1:500), goat anti-rabbit IgG Alexa 555 (1:500), and goat anti-mouse IgG Alexa 647 (1:500) (Invitrogen, Grand Island, N.Y.). Terminal deoxynucleotidyl transferase UTP nick end labeling (TUNEL) was done using ApopTag® Red In Situ Apoptosis Detection Kit (Millipore, Billerica, Mass.). Whole embryos images captured using a Leica MZ16A fluorescence stereomicroscope, a MicroPublisher 3.3 RTV digital camera, and Q-Capture software version 3.1.2 (QImaging, Surrey, BC, Canada). Sections visualized using a Leica DM6000 B upright fluorescence light microscope (Leica Microsystems, Bannockburn, Ill.), Retiga-SRV camera (Q-Imaging), and Volocity software version 6.3 (PerkinElmer, Walham, Mass.). All images prepared for publication using Photoshop and Illustrator version CS6 (Adobe System, Inc., San Jose, Calif.).

### Example 1—Tbx3 is Sufficient to Specify Pluripotent Cells to a Retinal Lineage in the Context of the Eye Field

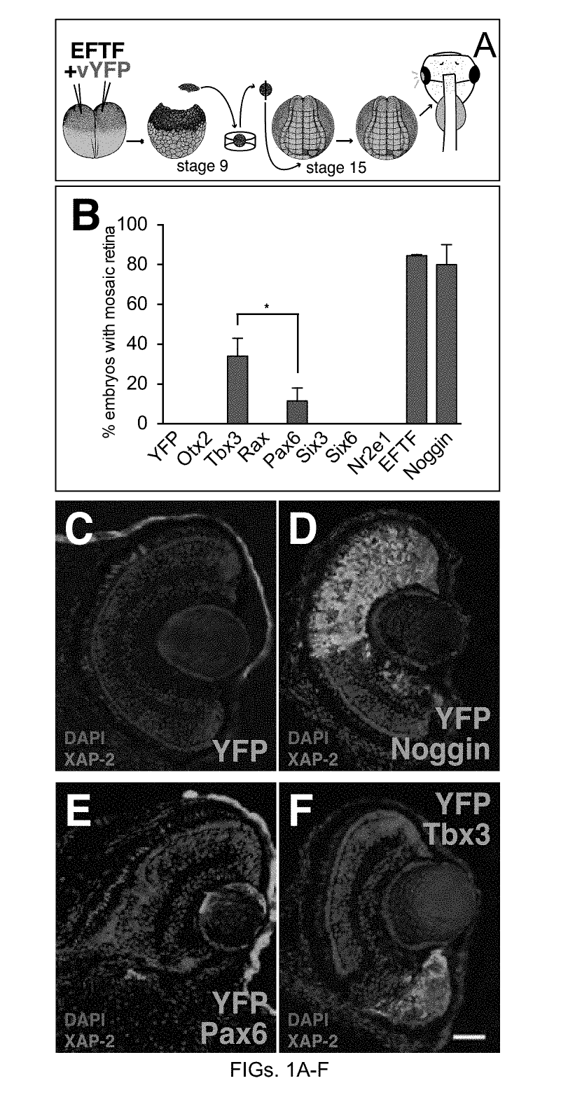

To determine which EFTFs could specify retina, we injected both blastomeres of 2-cell staged embryos and transplanted donor animal cap cells expressing venus YFP (vYFP) and individual EFTF to the stage 15 eye field of host embryos, sectioned the resulting retinas and stained for the rod photoreceptor marker, XAP-2 (ACT→EF, FIG. 1A) (Viczian et al., “Generation of Functional Eyes from Pluripotent Cells,” PLoS Biol 7:e1000174 (2009); Viczian et al., “Tissue Determination using the Animal Cap Transplant (ACT) Assay in Xenopus Laevis,” J Vis Exp 39:1932 (2010); Harris et al., “Two Cellular Inductions Involved in Photoreceptor Determination in the Xenopus Retina,” Neuron 9:357-372 (1992), which are hereby incorporated by reference in their entirety). Both noggin (nog) and the complete EFTF cocktail (otx2 and the EFTFs tbx3, pax6, rax, six3, six6, and nr2e1), efficiently specified retina (FIGS. 1B,D; noggin 80%, n=40; EFTF cocktail 83%, n=40). By contrast, transplanted cells isolated from embryos expressing vYFP only, or vYFP with Otx2, Rax, Six3, Six6 or Nr2e1, only formed epidermis (FIGS. 1B,C and not shown; n=minimum 40 each). Only tbx3 andpax6 were sufficient to specify retinal cells (FIGS. 1B,E,F). The number of embryos with donor cells forming retina was greater with tbx3 than pax6 (FIG. 1B, tbx3 35%, n=43; pax6 12%, n=42, P=0.023). Taken together, these results indicate that only Tbx3 and Pax6 are sufficient to specify pluripotent cells to a retinal lineage in the context of the eye field (Zuber et al., “Specification of the Vertebrate Eye by a Network of Eye Field Transcription Factors,” Development 130:5155-5167 (2003), which is hereby incorporated by reference in its entirety).

### Example 2—Tbx3 is Expressed in a Pattern Consistent with a Role in Eye Field Specification

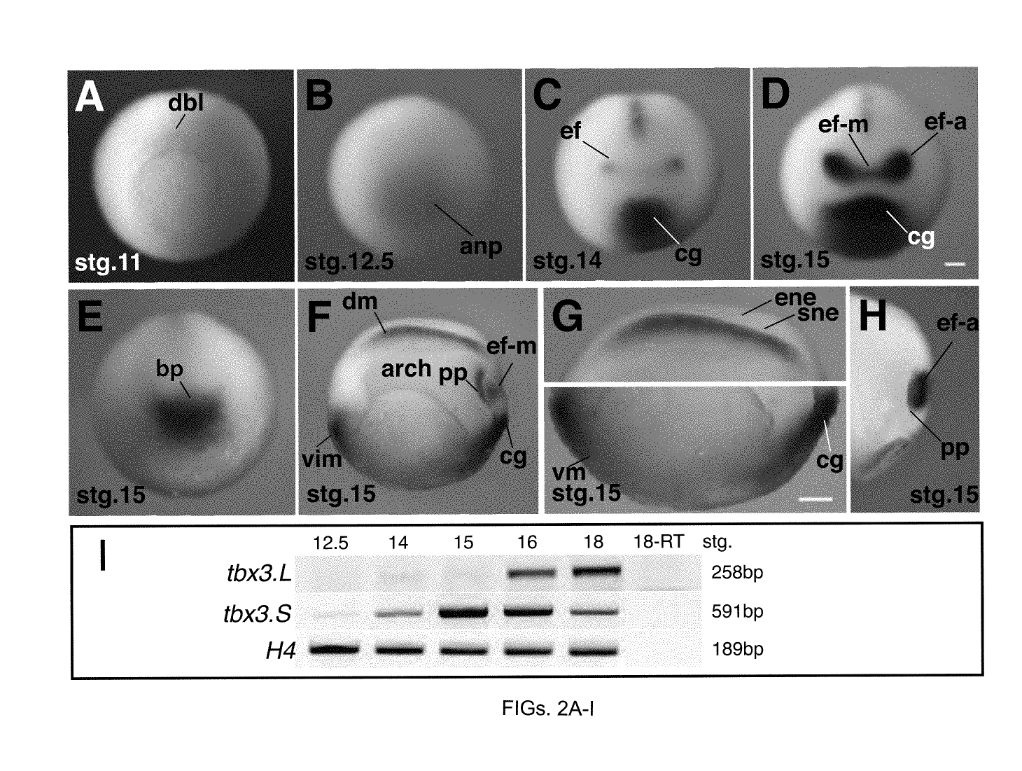

Previous reports indicated tbx3 is expressed in the anterior neural plate at eye field stages (Li et al., “A Single Morphogenetic Field Gives Rise to Two Retina Primordia under the Influence of the Prechordal Plate,” Development 124:603-615 (1997); Wong et al., “Efficient Retina Formation Requires Suppression of Both Activin and BMP Signaling Pathways in Pluripotent Cells,” Biol Open 4:573-583 (2015); and Zuber et al., “Specification of the Vertebrate Eye by a Network of Eye Field Transcription Factors,” Development 130:5155-5167 (2003), which are hereby incorporated by reference in their entirety). To more precisely define the tbx3 expression pattern, transcripts were detected by in situ hybridization (FIGS. 2A-I). Tbx3 was first detected in the dorsal blastopore lip at stage 11 (FIG. 2A). At yolk plug stage (stg. 12.5), diffuse expression was observed in the anterior neural plate (FIG. 2B). By stage 14, eye field and cement gland expression domains were distinct, and by stage 15, these domains were expanded, consistent with the pattern observed in previous reports at this stage (compare FIGS. 2C,2D; (Li et al., “A Single Morphogenetic Field Gives Rise to Two Retina Primordia under the Influence of the Prechordal Plate,” Development 124:603-615 (1997); Takabatake et al., “Conserved Expression Control and Shared Activity Between Cognate T-box Genes Tbx2 and Tbx3 in Connection with Sonic Hedgehog Signaling During Xenopus Eye Development,” Dev Growth Differ 44:257-271 (2002), which are hereby incorporated by reference in their entirety). At stage 15, expression of tbx3 extends into the ventral mesoderm and epidermal ectoderm from both the posterior blastopore and the anterior cement gland (FIGS. 2E,F,G). In mid-sagittal sections, expression is in the dorsal (somitogenic) mesoderm, but absent from the archenteron roof and epithelial and sensorial layers of the dorsal neuroectoderm (FIGS. 2F,G). Prechordal plate expression is detected both in the midline and immediately beneath the eye anlagen. Eye field expression is reduced at the midline but strongest in the eye anlagen (FIGS. 2D,F,H). Xenopus laevis is a pseudo-tetraploid with two tbx3 genes (homeologs) named tbx3.L and tbx3.S based on their sub-genome location (long and short chromosomes, respectively) (Bisbee et al., “Albumin Phylogeny for Clawed Grogs (Xenopus),” Science 195:785-787 (1977), which is hereby incorporated by references in its entirety). To determine if both are expressed in the developing eye, eye field RNA was subjected to RT-PCR, using homeolog-specific primers. Both homeologs were present with tbx3.S (stg. 12.5) detected prior to tbx3.L (stg. 14, FIG. 2I). Together, these results indicate tbx3 is expressed in a pattern consistent with a role in eye field specification and both homeologs are expressed in the developing eye.

### Example 3—Tbx3 is Required for Normal Eye Formation

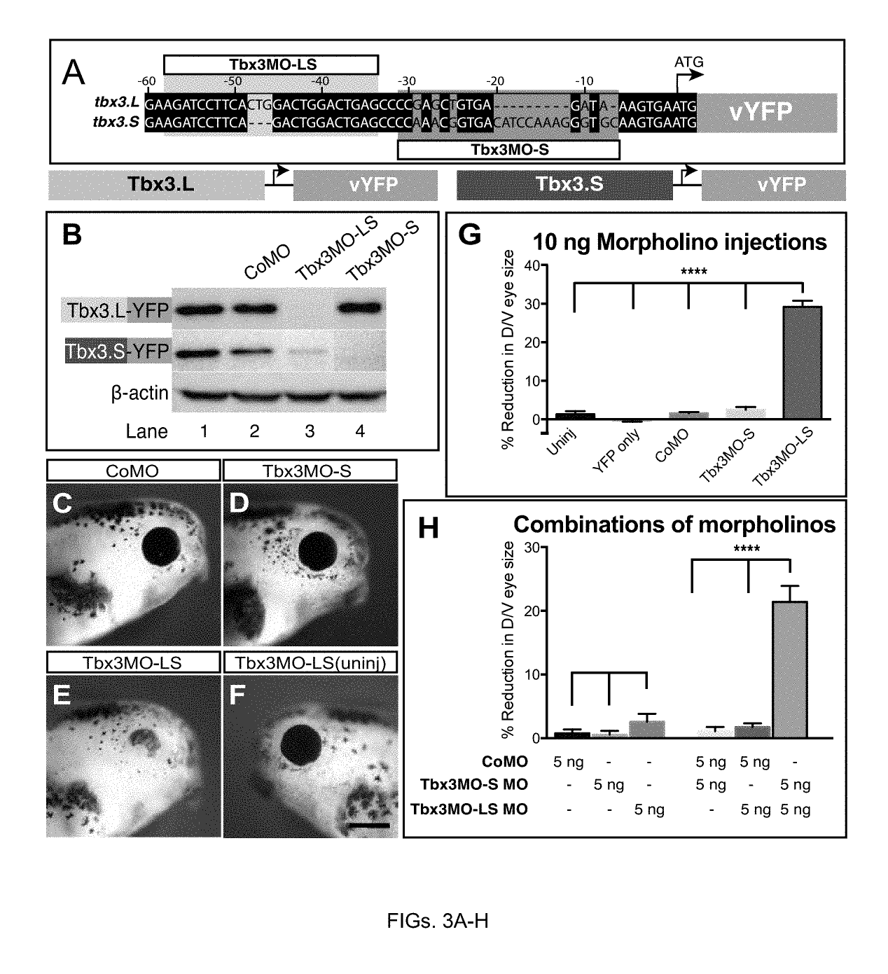

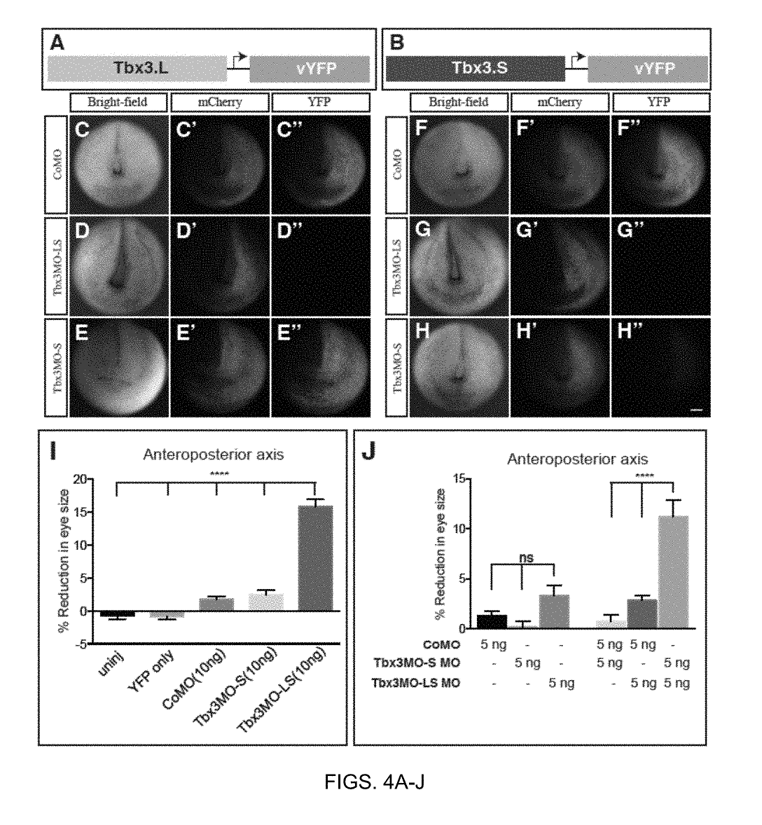

To determine if tbx3 is required for normal eye formation, tbx3-specific morpholinos (MOs) were used in knockdown experiments. Tbx3MO-LS targets a sequence predicted to inhibit translation of both tbx3 homeologs (FIGS. 3A, 4A), while Tbx3MO-S only targets tbx3.S (FIGS. 3A, 4B). Antibodies recognizing X. laevis Tbx3 are not available, therefore fusion constructs were generated to test the translation blocking ability of the morpholinos (FIGS. 3B, 4C-H″). As predicted, Tbx3MO-LS inhibited translation of both Tbx3.L and Tbx3.S, while Tbx3MO-S only inhibited Tbx3.S expression, as determined by both fusion protein fluorescence in vivo and Western blot analysis (FIGS. 3B, 4C-H″).

Embryos unilaterally injected into one dorsal blastomere (D1) at the 8-cell stage with morpholinos were grown to tadpoles for analysis (stage 43; FIGS. 3C-F). The eye on the injected side of tadpoles treated with 10 ng of CoMO or Tbx3MO-S morpholino were indistinguishable from control, wild-type embryos (FIG. 3C, n=195; FIG. 3D, n=80). In contrast, injection with 10 ng of Tbx3MO-LS morpholino reduced eye size in 94% of tadpoles (FIG. 3E n=191). The dorsoventral eye diameter of the injected and uninjected side of tadpoles was compared. Eye size varied little in wild-type, uninjected tadpoles (FIG. 3G, 1.4±0.8%, n=30), or embryos injected with vYFP, CoMO or Tbx3MO-S (FIG. 3G, YFP-only; −0.3±0.3%, n=95; CoMO, 1.6±0.3%, n=195; Tbx3MO-S, 2.5±0.7%, n=80). In contrast, knockdown with Tbx3MO-LS reduced dorsoventral eye diameter by 29.2±1.6% (FIG. 3G, n=191). Similar effects were observed when the anteroposterior eye diameter was measured (FIG. 41). Injection into non-retinogenic blastomeres however, did not alter eye formation (V1, 0%, n=65; D2, 0%, n=58; V2, 0%, n=63).

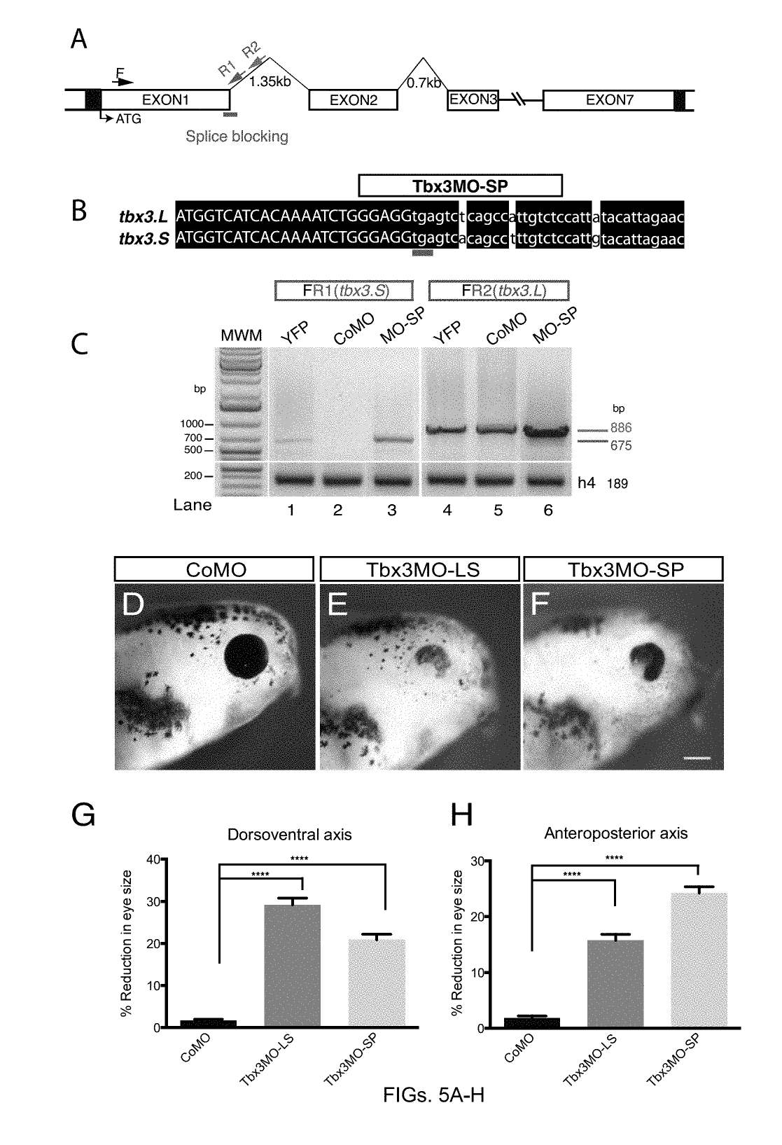

To determine if both homeologs were required, morpholinos were coinjected at suboptimal levels. When injected individually, 5 ng did not alter eye size significantly (FIG. 3H, CoMO, 0.8±0.6%, n=59; Tbx3MO-S, 0.6±0.6%, n=43; Tbx3MO-LS, 2.6±1.2%, n=29). Coinjection of CoMO with Tbx3MO-S or Tbx3MO-LS (10 ng total) did not alter eye size either (FIG. 3H, Tbx3MO-S+CoMO, 1.1±0.6%, n=41; Tbx3MO-LS+CoMO, 1.8±0.6%, n=52). However, Tbx3MO-S and Tbx3MO-LS together synergistically reduced both the dorsoventral and anteroposterior eye diameter relative to controls (FIG. 3H, DV 21.4±1.6% n=135; FIG. 4J, AP 11.2±1.6%, n=135). To confirm the reduction in eye size was due to eye field-specific reduction of Tbx3, Tbx3MO-LS was injected into the most retinogenic dorsal blastomeres of 16- and 32-cell staged embryos (Moody, S. A., “Fates of the Blastomeres of the 32-Cell-Stage Xenopus Embryo,” Dev Biol 122: 300-319 (1987a); Moody, S. A., “Fates of the Blastomeres of the 16-Cell Stage Xenopus Embryo,” Dev Biol 119:560-578 (1987b); Huang et al., “The Retinal Fate of Xenopus Cleavage Stage Progenitors is Dependent upon Blastomere Position and Competence: Studies of Normal and Regulated Clones,” J Neurosci 13:3193-3210 (1993), which are hereby incorporated by reference in their entirety). Tbx3MO-LS reduced eye size in 57% of embryos injected into blastomere D1.1 at the 16-cell stage (n=35, not shown) and 75% of embryos injected in D1.1.1 at the 32-cell stage (n=24). Finally, as an independent test to confirm eye defects were through Tbx3 loss, we also generated a morpholino (Tbx3MO-SP) targeting the exon 1 splice donor sites of both tbx3.L and tbx3.S, resulting in an in-frame stop codon in the unspliced transcripts (FIGS. 5A,B). As determined by PCR, injection of Tbx3MOSP increased the amount of unspliced tbx3.L and tbx3.S transcripts and resulted in eye defects similar to those observed with Tbx3MO-LS (FIGS. 5C-H). Together, these results indicate the eye field expression of Tbx3 is required for normal eye formation, and either Tbx3.L or Tbx3. S may be sufficient for eye formation.

### Example 4—Tbx3 is Required for the Retinal and Neural Inducing Activity of Noggin

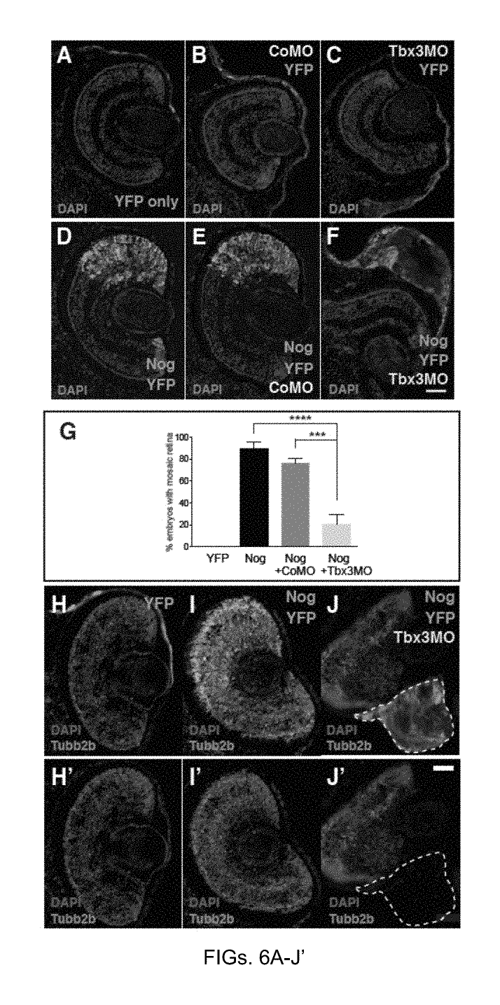

Noggin can specify pluripotent cells to a retinal fate (FIGS. 1A-F) (Wong et al., “Efficient Retina Formation Requires Suppression of Both Activin and BMP Signaling Pathways in Pluripotent Cells,” Biol Open 4:573-583 (2015); Viczian et al., “Generation of Functional Eyes from Pluripotent Cells,” PLoS Biol 7:e1000174 (2009); Lan et al., “Noggin Elicits Retinal Fate in Xenopus Animal Cap Embryonic Stem Cells,” Stem Cells 27:2146-2152 (2009), which are hereby incorporated by reference in their entirety). To determine if Tbx3 knockdown altered the retina specifying activity of Noggin, the experiments described with respect to FIGS. 1A-F were repeated, but Tbx3MO-LS (for simplicity referred to as Tbx3MO from here on) was coinjected with Noggin and whether cells formed retina (ACT→EF) was determined. Cells injected with YFP, CoMO, or Tbx3MO never formed retina (FIGS. 6A-C; YFP, 0% n=40; CoMO 0% n=40; Tbx3MO, 0% n=52). Transplantation of donor cells expressing Noggin, however, generated mosaic retinas in 89% of animals (FIGS. 6D,G; n=78). While co-injection of CoMO did not alter retina-inducing activity of Noggin (FIGS. 6E,G; 76% n=82, P=0.32), Tbx3MO reduced the number of embryos with YFP+mosaic retinas significantly (FIGS. 6F,G; 22% n=73). To determine if Tbx3MO blocked both the neural, as well as retinal, inducing activity of Noggin, retinal tissue was stained with the neural marker Class II β-tubulin (Tubb2b) (Moody et al., “Developmental Expression of a Neuron-Specific Beta-Tubulin in Frog (Xenopus laevis): A Marker for Growing Axons During the Embryonic Period,” J Comp Neurol 364:219-230 (1996), which is hereby incorporated by reference in its entirety). Tubb2b protein was detected in the inner and outer plexiform layers (FIGS. 6H,H′, n=40). The processes of retinal neurons generated from Noggin-expressing pluripotent cells always expressed Tubb2b (FIG. 41,I′; 100% Tubb2b+/YFP+ transplants, n=45). Surprisingly, Tbx3MO, dramatically reduced the expression of Tubb2b in transplants derived from Noggin-expressing cells (FIGS. 6J,J′; 4% Tubb2b+/YFP+ transplants, n=45). Together, these results suggest Tbx3 is required for the ability of Noggin to specify pluripotent cells to both a retinal and neural fate in the context of the eye field.

### Example 5—Tbx3 is a Neural Inducer, but Unlike Noggin, is not Sufficient to Determine Pluripotent Cells to a Retinal Lineage

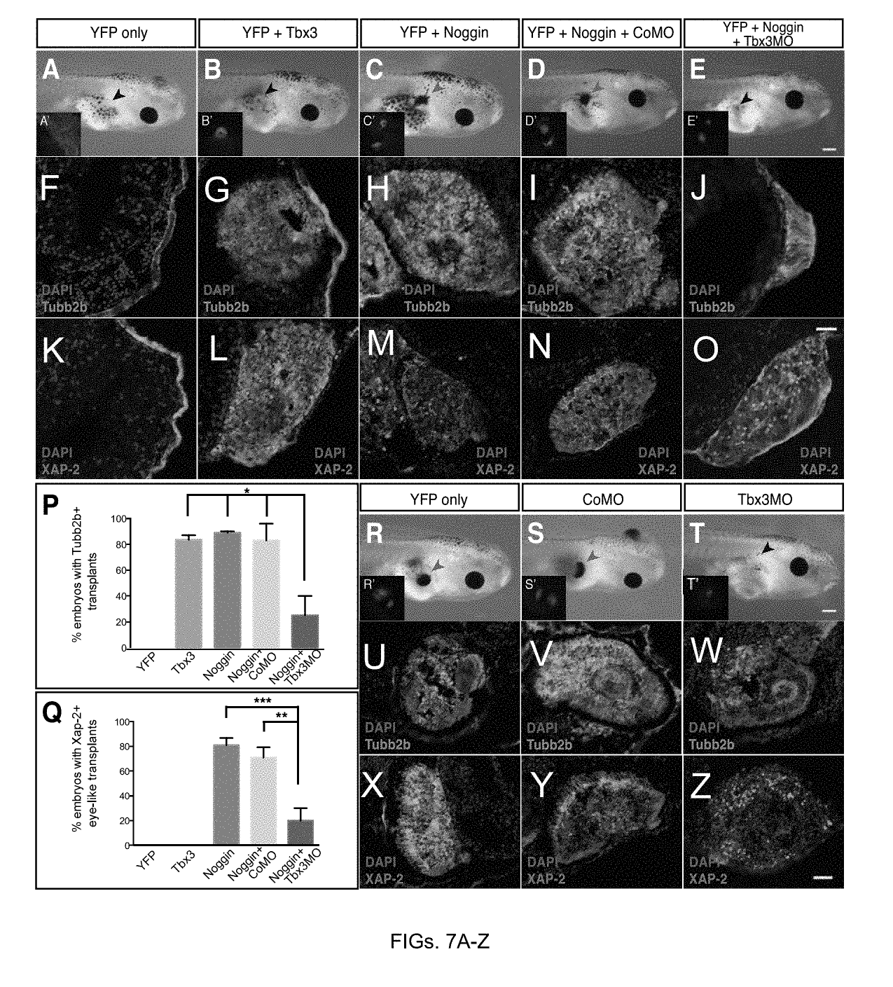

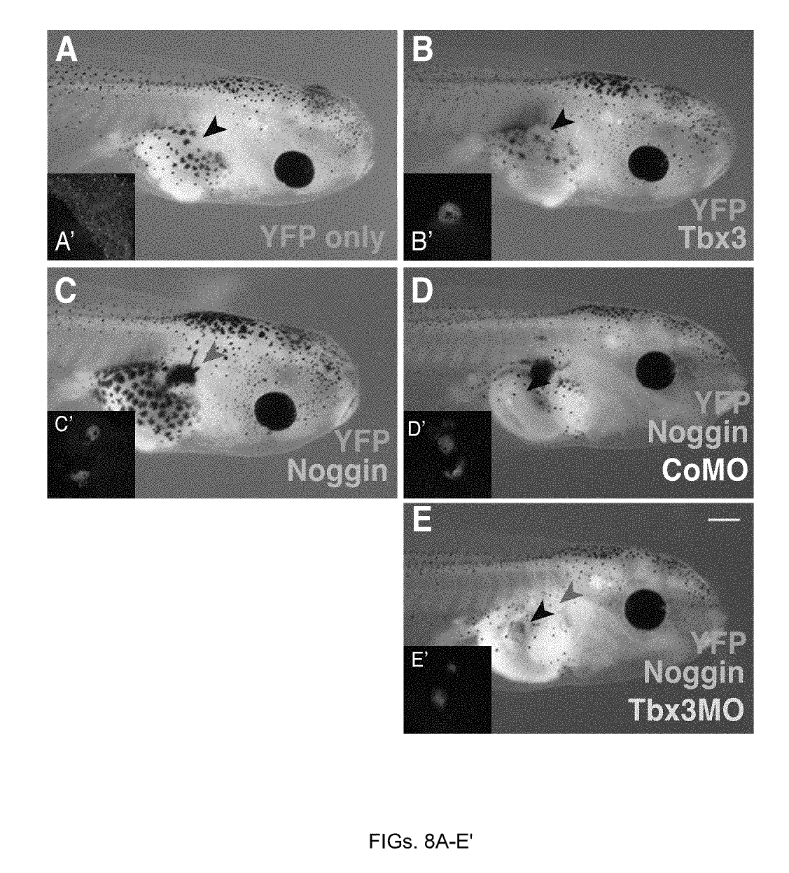

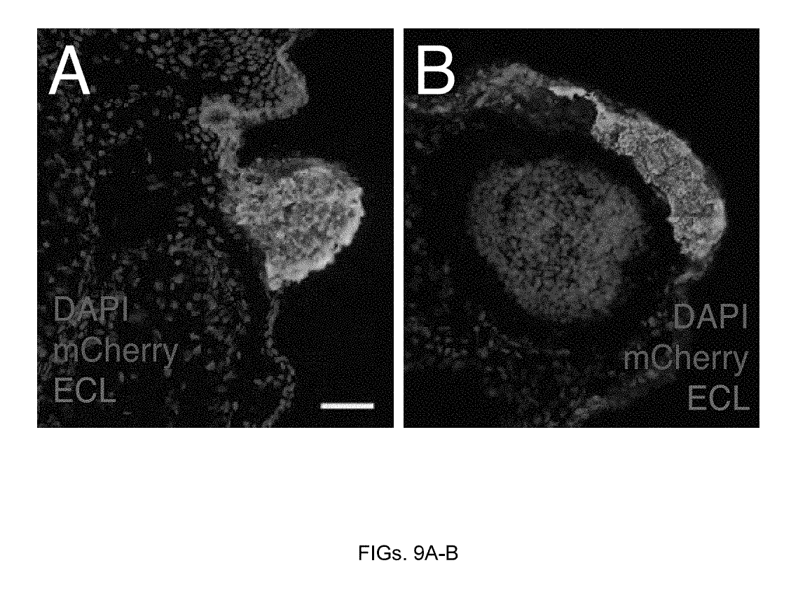

To further test the hypothesis that Tbx3 is required for both the neural and retinal inducing activity of Noggin, animal cap donor cells were transplanted to the flank of stage 15 host embryos (ACT→Flank), which were then grown to tadpoles. YFP-expressing donor cells only generated epidermis (FIGS. 7A-A′,F,K; 100% n=80; FIG. S3). Cells isolated from embryos injected with tbx3 expressed the neural marker Tubb2b in 83% of transplants (FIG. 7G, n=55), but never the rod photoreceptor marker XAP-2 (FIGS. 7L,Q, 0%, n=50). Noggin-expressing controls generated ectopic eye-like structures in 35% (YFP) and 33% (YFP+CoMO) of donor transplants (FIGS. 7C-D′, also see FIGS. 8A-AA), which expressed both neural, Tubb2b (FIGS. 7H,P, 89%, n=41) and rod, XAP-2, markers (FIGS. 7M,Q, 81%, n=100), and had a morphology consistent with retina formation (FIGS. 7M,N). In contrast, donor cells with Noggin and Tbx3MO formed a more lightly pigmented tissue mass suggesting Tbx3 knockdown resulted in a change from a neural and retinal, to cement gland fate (FIGS. 7E,J,O, 70%, n=79, see also FIGS. 8A-E′). Consistent with this interpretation transplants expressed a cement gland marker, Erythrina cristagalli Lectin (ECL) (Turton et al., “Crystal Structures of Erythrina cristagalli lectin with Bound N-Linked Oligosaccharide and Lactose,” Glycobiology 14:923-929 (2004), which is hereby incorporated by reference in its entirety) (FIGS. 9A-B). Therefore, Tbx3 knockdown significantly reduced the ability of Noggin to induce both neural (FIGS. 7J,P; Tubb2b 25% n=39) and retinal markers (FIGS. 7O,Q; XAP-2 20% n=40), resulting in the cells taking on a more anterior, non-neural cement gland fate.

To determine whether Tbx3 knockdown would have the same effect on genuine (rather than Noggin-induced) eye field cells (EF-Flank), stage 15 eye field cells from embryos injected in one blastomere at the 8-cell stage with YFP alone, with CoMO, or with Tbx3MO were transplanted to the flank of host embryos. Eye field fragments isolated from YFP-only or CoMO-injected embryos formed ectopic eyes, including retinal pigmented epithelium (RPE), in 90% and 85% of flank transplants, respectively (YFPonly: FIG. 7R; FIG. 7U, Tubb2b 100%, n=13; FIG. 7X, XAP-2 100%, n=9; YFP and CoMO: FIG. 7S; FIG. 7V, Tubb2b 100%, n=9; FIG. 7Y, XAP-2 100%, n=12). In contrast, YFP-positive donor eye field cells from Tbx3MO-injected embryos were never pigmented nor laminated (FIGS. 7T,T′, n=19). Only 27% and 25% of the structures expressed Tubb2b or XAP-2, respectively (YFP and Tbx3MO: FIG. 7W, n=1 and FIG. 7Z, n=8). Transplanted cells were disorganized and regions expressing either Tubb2b or XAP-2 were YFP-negative. Eight-cell stage injection labels most, but not all donor eye field cells, therefore YFP-negative regions were most likely originated from donor eye field cells that did not receive Tbx3MO. These results suggest Tbx3 is a neural inducer, sufficient to determine pluripotent cells to a neural, but not retinal lineage. Furthermore, Noggin requires Tbx3 to generate both neural and retinal tissues from pluripotent cells.

### Example 6—Tbx3 Specifies Spinal Cord but not Retina, while Noggin Expressing Cells Remain Determined to Form Retina in Posterior Neural Plate Transplants

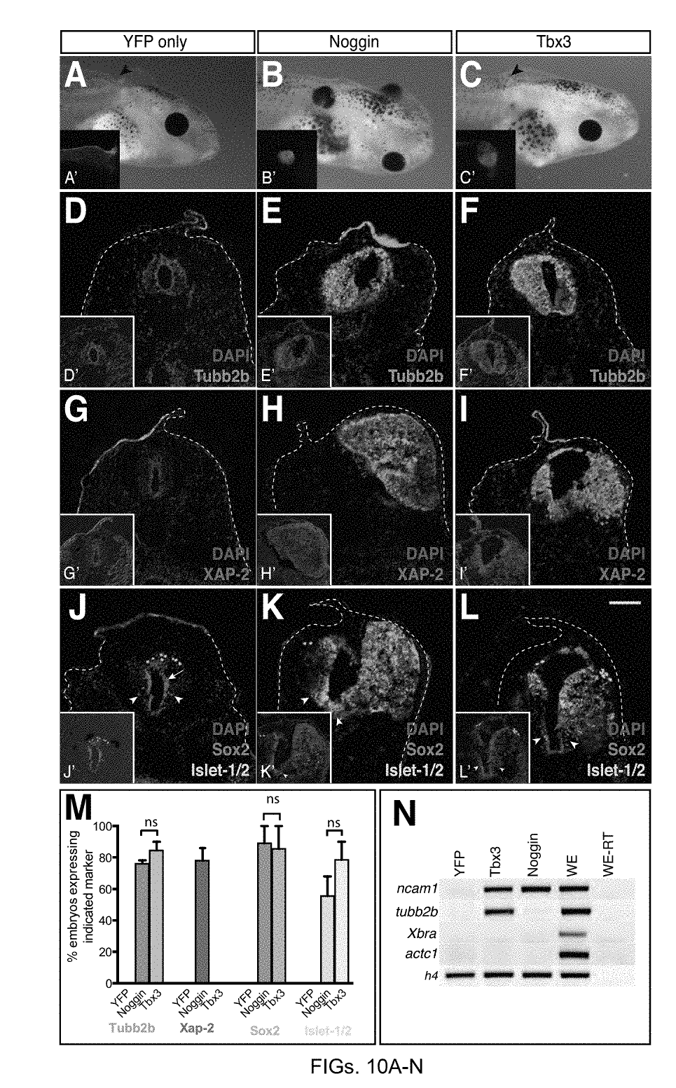

Tbx3 expressing cells formed retina when transplanted to the stage 15 eye field, but not in the stage 15 flank. One possible explanation for this difference is the neural plate provides a factor(s) necessary for retina formation that is not present in the flank. To test this idea, ectodermal explants were generated as before, but Tbx3 expressing cells were transplanted to the stage 15 posterior neural plate instead (ACT→PNP). Embryos were grown to tadpoles and sections containing the donor cells were stained for the presence of retinal markers. YFP-expressing cells only generated epidermis (FIGS. 10A,A′,D,G,J,M, 100%; n=78), when transplanted to the posterior neural plate (ACT→PNP). Cells expressing Noggin generated ectopic eye-like structures in 61% of the transplants (FIGS. 10B,B′; n=92 red arrowhead). Tbx3-expressing donor cells, however, never generated ectopic eyelike structures (FIGS. 10C,C′; n=108 black arrowhead). To determine the differentiated fate of donor cells, sectioned embryos were stained with antibodies recognizing neural, retinal, and spinal cord markers. Expression of Tubb2b in controls stains the bilaterally symmetrical spinal cord (FIGS. 10D,D′). In addition to ectopic eye-like structures, Noggin expressing donor cells were also detected in the enlarged Tubb2b-expressing spinal cord (76%, n=33), and often distorted the normal symmetry of the tissue (FIGS. 10E,E′,M). Although no ectopic eyes were detected in tadpoles that received transplants expressing Tbx3, the spinal cord of 88% were mosaic (n=108), and 86% expressed Tubb2b (FIGS. 10F,F′,M, n=35). Noggin-expressing donor cells expressed XAP-2, and rod photoreceptor outer segments in 76% of transplants (FIGS. 10H,M, n=34). Despite being transplanted to the neural plate, Tbx3 expressing cells never expressed XAP-2 and no evidence of RPE, rod outer segments or lamination were detected (FIGS. 10I,M, n=32).

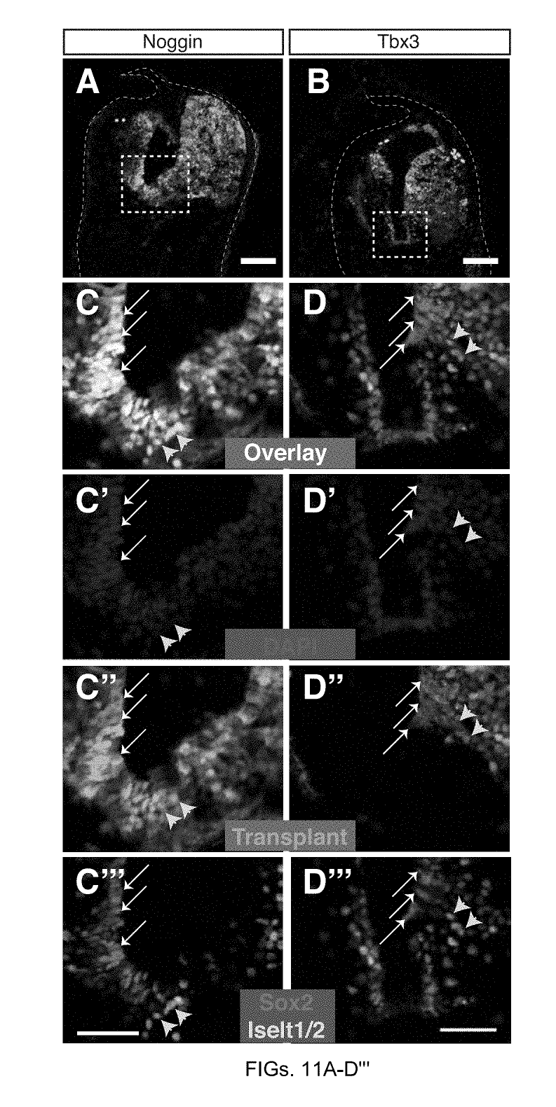

To determine if transplanted tissues were being specified to spinal cord, tissue was stained for Sox2 and Islet proteins (FIGS. 10J-L). In the spinal cord, Sox2 is expressed in the ventricular zone of the spinal cord (FIGS. 10J,J′) (Gaete et al., “Spinal Cord Regeneration in Xenopus Tadpoles Proceeds through Activation of Sox2-Positive Cells,” Neural Dev 7:13 (2012), which is hereby incorporated by reference in its entirety). Islet-1/2 expressing cells are detected in ventral post-mitotic motor neurons (MN) and dorsal Rohon-Beard cells (FIGS. 6J,J′, FIG. 11) (Diez del Corral et al., “Markers in Vertebrate Neurogenesis,” Nat Rev Neurosci 2:835-839 (2001); Yajima et al., “Six1 is a Key Regulator of the Developmental and Evolutionary Architecture of Sensory Neurons in Craniates,” BMC Biol 12:40 (2014); Olesnicky et al., “prdm1a Regulates sox 10 and islet1 in the Development of Neural Crest and Rohon-Beard Sensory Neurons,” Genesis 48:656-666 (2010), which are hereby incorporated by reference in their entirety). In addition, the anti-Islet-1/2 antibody labels subpopulations of ganglion, amacrine, bipolar, and horizontal cells in the tadpole retina (Álvarez-Hernán et al., “Islet-1 Immunoreactivity in the Developing Retina of Xenopus Laevis,” Scientific World Journal 740420 (2013), which is hereby incorporated by reference in its entirety). Noggin-expressing, YFP-positive donor cells were co-labelled with both Sox2 and Islet-1/2 antibodies in 91% and 57% of transplants, respectively (FIGS. 10K,K′,M, FIG. 11, n=33). Islet-1/2-expressing cells were detected at positions consistent with the location of motor neurons, but also throughout the majority of the donor tissue (FIGS. 10K,K′, FIG. 11). Expression of the rod photoreceptor marker in these same regions (FIG. 10H) suggests the majority of the stained cells distant from the midline may be retinal ganglion, amacrine, bipolar, and/or horizontal cells. Donor cells expressing Tbx3 also expressed Sox2 and Islet-1/2, but in a more restricted expression pattern consistent with the expected location of spinal neurons. YFP+/Sox2+ cells were detected in the ventricular zone in 85% of transplants, while YFP+/Islet-1/2+ cells (78% of transplants) were observed in regions consistent with the location of the ventral motor neurons (FIGS. 10L,L′,M, FIG. 11, n=41).

To determine if grafting of the cells into an embryo was required for Tbx3 to induce neural markers, Tbx3 expressing explants were grown in culture and RT-PCR was used to detect the expression of the markers neural cell adhesion molecule 1 (ncam1) and tubb2b. Tbx3 was sufficient to induce expression of both ncam1 and tubb2b, while Noggin only strongly induced ncam1 (FIG. 10N). To determine if Tbx3 induced neural markers directly, or indirectly through mesoderm induction, RT-PCR was also used to detect the expression the pan-mesodermal marker xbra, and the dorsal mesoderm marker actin, alpha, cardiac muscle 1, actc1. Neither Noggin, nor Tbx3 induced mesodermal markers indicating both are direct neural inducers (FIG. 10N).

Together, these results indicate Tbx3, like Noggin, induces neural tissue directly. However, unlike Noggin, Tbx3 is unable to determine pluripotent cells to a retinal lineage outside the eye field, even when cells are transplanted to other regions of the neural plate (ACT→PNP). The location dependent specification of Tbx3 expressing cells (ACT→EF→retina versus ACT→PNP→spinal cord) suggests Tbx3 may maintain neural progenitors in a multipotent state, and as yet unknown contextual cues, dictate the eventual differentiated fate of the cells.

### Example 7—Tbx3 Represses bmp4 Expression in Pluripotent Cells and the Anterior Neural Plate During Eye Field Specification

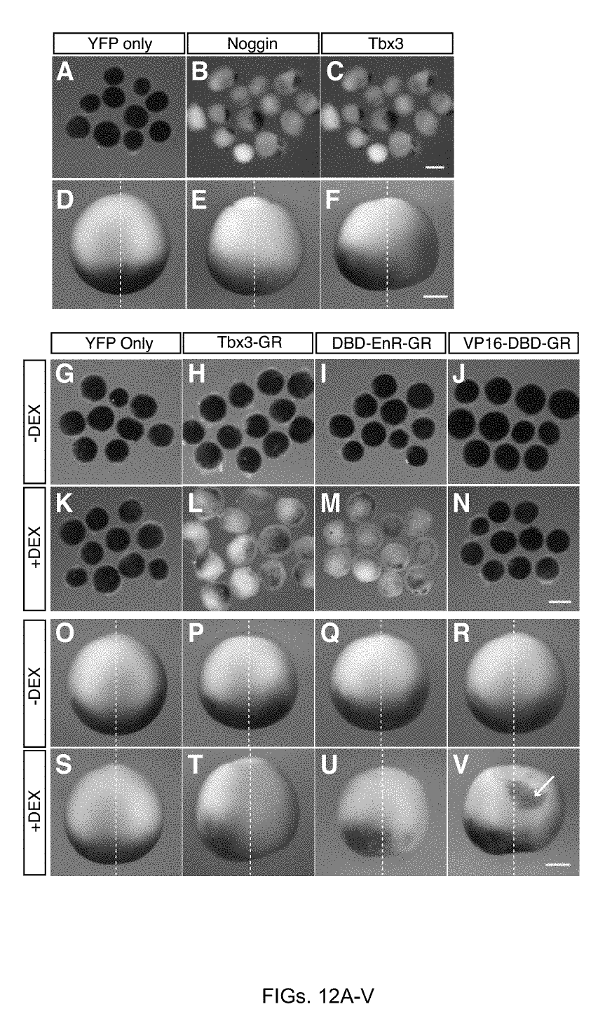

Noggin can repress bmp4 expression. Since Tbx3 is required for the neural and retinal inducing activity of Noggin, and both Noggin and Tbx3 are neural inducers, we asked if Tbx3 could repress bmp4. All YFP-expressing explants express bmp4 (FIG. 12A, n=92). In contrast, bmp4 expression was reduced in explants expressing either Noggin or Tbx3 (FIG. 12B, 93%, n=62 and FIG. 12C, 86%, n=88). Prior to gastrulation, bmp4 expression is detected in the dorsal ectoderm (future neural plate) but by stage 12.5, expression is excluded from the neural plate and detected in more anterior and ventrolateral regions of the embryo (FIG. 12D). Unilateral expression of either Noggin or Tbx3 reduced bmp4 expression on the injected side of embryos (FIG. 12E, 20%, n=126 and FIG. 12F, 83%, n=153).

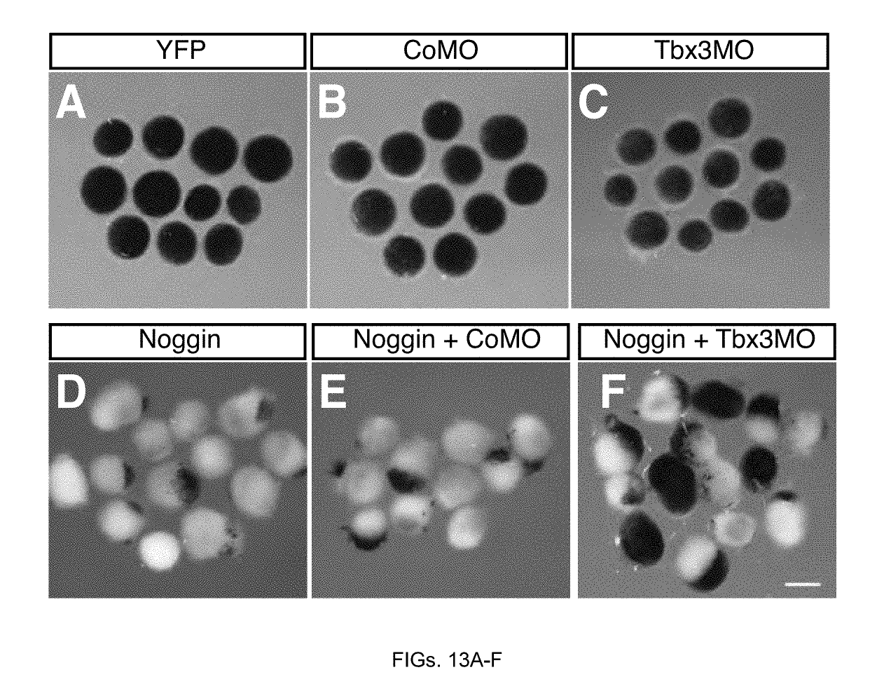

To determine if the ability of Noggin to repress bmp4 expression is also dependent on Tbx3, ectodermal explants were isolated from embryos expressing Noggin in the presence or absence of Tbx3 morpholinos. Neither control morpholino nor Tbx3MO alone altered the expression of bmp4 relative to YFP expressing explants (FIGS. 13A-C). Noggin repressed bmp4 expression in 91% and 81% of explants when expressed alone or with control morpholino, respectively (FIG. 13D, n=47 and FIG. S6E, n=48). When Noggin was injected with Tbx3MO however, bmp4 expression recovered, being repressed in only 37% of explants (FIG. 13F, n=49) indicating Tbx3 is also necessary for the ability of Noggin to repress bmp4 expression.

Although Tbx3 was initially reported to be a transcriptional repressor, it can also function as an activator (He et al., “Transcription Repression by Xenopus ET and its Human Ortholog TBX3, a Gene Involved in Ulnar-Mammary Syndrome,” Proc Natl Acad Sci USA 96:10212-10217 (1999); Carlson et al., “A Dominant Repression Domain in Tbx3 Mediates Transcriptional Repression and Cell Immortalization: Relevance to Mutations in Tbx3 that Cause Ulnar-Mammary Syndrome” Hum Mol Genet 10:2403-2413 (2001); Lu et al., “Dual Functions of T-box 3 (Tbx3) in the Control of Self-Renewal and Extraembryonic Endoderm Differentiation in Mouse Embryonic Stem Cells,” J Biol Chem 286:8425-8436 (2011), which are hereby incorporated by reference in their entirety). To determine how Tbx3 regulates bmp4 expression, repressor and activator versions were generated to determine how they altered bmp4 expression. Tbx3 is expressed prior to eye field stages (FIGS. 2A-I). To avoid disrupting possible earlier roles of Tbx3 function, hormone inducible version were generated using the ligand binding domain of the glucocorticoid receptor (GR) and activated by dexamethasone treatment starting at stage 9 (Kolm et al., “Efficient Hormone-Inducible Protein Function in Xenopus Laevis,” Dev Biol 171:267-272 (1995), which is hereby incorporated by reference in its entirety). Dexamethasone did not alter bmp4 expression in explants of pluripotent cells (compare FIG. 12G, n=73 and FIG. 12K, n=50). Fusion of the entire coding region of Tbx3 to GR (Tbx3-GR) rendered Tbx3 activity dexamethasone dependent. Bmp4 expression was unaltered in Tbx3-GR expressing cells (FIG. 12H, n=36), but hormone treatment reduced expression in 87% of explants (FIG. 12L, n=40). Similar results were obtained when only the DNA binding domain of Tbx3 (DBD) was fused to the engrailed repressor domain and GR (DBD-EnR-GR). In explants expressing DBD-EnR-GR, bmp4 expression was reduced in only 8% of explants (FIG. 12I, n=49), but increased to 91% when treated with hormone (FIG. 12M, n=46). No change in bmp4 expression was detected when Tbx3 was fused to the transactivation domain of VP16 (VP16-DBD-GR) (Takabatake et al., “Conserved Expression Control and Shared Activity Between Cognate T-box Genes Tbx2 and Tbx3 in Connection with Sonic Hedgehog Signaling During Xenopus Eye Development,” Dev Growth Differ 44:257-271 (2002), which is hereby incorporated by reference in its entirety). Bmp4 was detected throughout explants with or without dexamethasone treatment (compare FIG. 12J, n=49 and FIG. 12N, n=55). Together, the above results suggest Tbx3 functions as a transcriptional repressor and is necessary for Noggin to repress bmp4 expression in cultured explants.

Whether Tbx3-GR, DBD-EnR-GR and VP16-DBD-GR would regulate bmp4 expression in vivo was investigated. Embryos were injected unilaterally, grown in hormone starting at stage 9 to activate the fusion constructs and processed at early eye field stage (12.5). Dexamethasone did not alter the expression pattern of bmp4 in YFP-injected embryos (compare FIG. 12O, n=62 to FIG. 12S, n=75). In contrast, the frequency of bmp4 repression was nearly 5-fold greater with hormone treatment in embryos expressing either Tbx3-GR (12%; n=78 to 58%; n=105) or DBD-EnR-GR (14%; n=55 to 75%; n=80). VP16-DBD-GR did not alter the expression pattern of bmp4 in any of the untreated embryos (FIG. 12R, n=48). In contrast to explants however, dexamethasone treatment dramatically altered the expression pattern of bmp4 (n=67). Ectopic expression was observed in the neural plate (100%) and reduced expression (73%) anterior to the neural plate (FIG. 12V). These results suggest, that both Noggin and Tbx3 repress bmp4 expression, and can do so both in isolated ectodermal explants, as well as in the anterior neural plate during eye field specification.

### Example 8—the Repressor Activity of Tbx3 is Required for Normal Neural Patterning During Eye Field Stages and Tbx3 Knockdown in Retinal Progenitors Results in Cell Death and Eye Defects

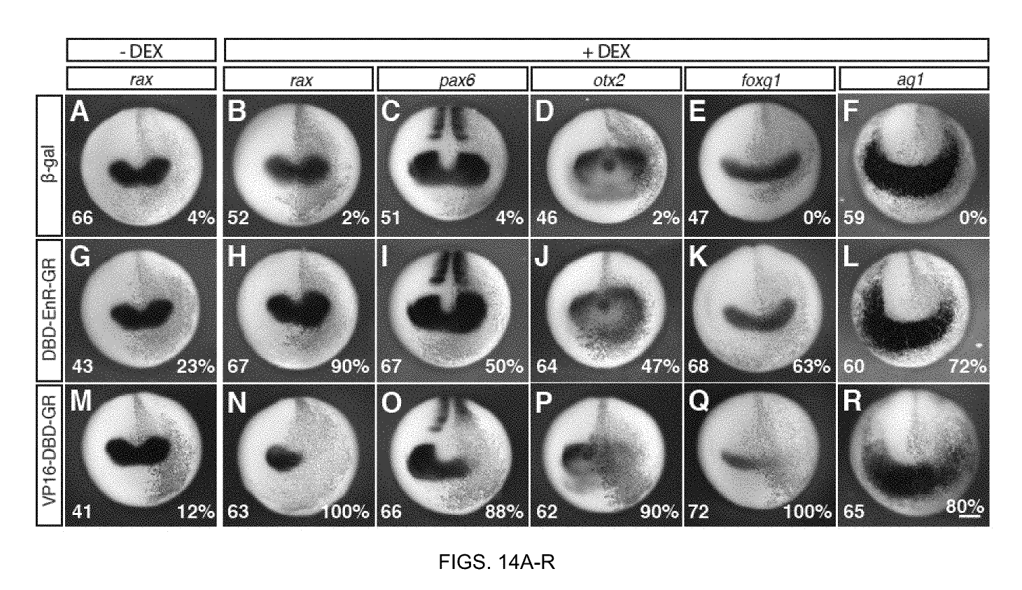

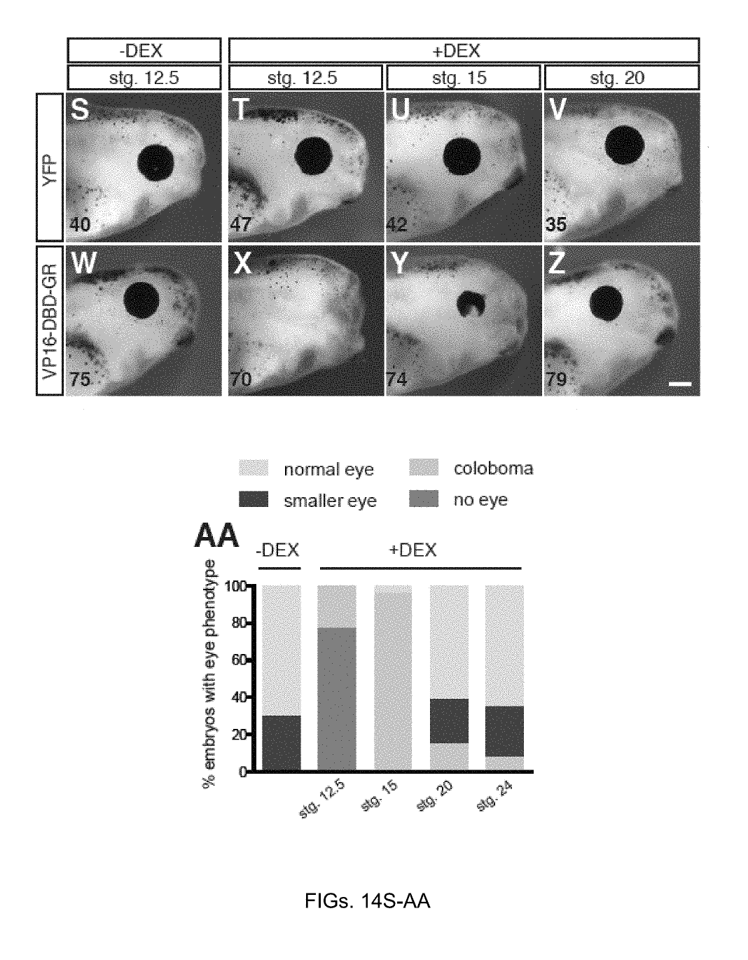

Tbx3 represses bmp4 transcription (FIGS. 12A-V) and continuous inhibition of BMP signaling is required for normal anterior neural development (Hartley et al., “Transgenic Xenopus Embryos Reveal that Anterior Neural Development Requires Continued Suppression of BMP Signaling After Gastrulation,” Dev Biol 238:168-184 (2001), which is hereby incorporated by reference in its entirety). To determine if normal anterior neural patterning is regulated by Tbx3 activity, the effects of DBD-EnR-GR and VP16-DBD-GR on eye field (rax andpax6), forebrain and midbrain (otx2), prospective telencephalon (foxg1), and cement gland (ag1) markers were determined (Mathers et al., “The Rx Homeobox Gene is Essential for Vertebrate Eye Development,” Nature 387:603-607 (1997); and Li et al., “A Single Morphogenetic Field Gives Rise to Two Retina Primordia under the Influence of the Prechordal Plate,” Development 124:603-615 (1997), which are hereby incorporated by reference in their entirety). In the absence of dexamethasone, marker expression patterns were unaltered (FIGS. 14A,G,M). Activation of DBD-EnR-GR by dexamethasone treatment starting at stage 12.5 however, resulted in an expansion of the rax, pax6, otx2 and to a lesser extend foxg1 expression domains (FIGS. 14H-K), while the expression domain of the cement gland marker ag1 was reduced in most embryos (FIG. 14L). Activation of VP16-DBD-GR had the opposite effect, since the rax, pax6, otx2 and foxg1 expression domains were either reduced or completely lost (FIGS. 14N-Q) and the ag1 expression domain appeared expanded and more diffuse in most embryos (FIG. 14R).

To determine if, and when, the repressor activity of Tbx3 was required for normal eye formation, the VP16-DBD-GR protein in embryos was activated at different time points, embryos were grown to tadpoles, and the effect on eye formation was determined. YFP alone had no detectable effect on eye formation and the eyes of embryos injected with YFP and VP16-DBD-GR were only slightly smaller on the injected side in some tadpoles (FIGS. 14S,W, and AA). By contrast, VP16-DBD-GR activation with dexamethasone starting at stage 12.5, resulted in eyeless embryos 77% and coloboma 23% of the time, respectively (FIGS. 14X, AA). The frequency and severity of eye defects was reduced when dexamethasone treatment was started at later developmental stages with relatively little effect on eye formation after eye field stages (FIGS. 14Y, Z and AA). From these results it was concluded that the repressor activity of Tbx3 is required at eye field stages (stg. 12.5-15) not only for reducing bmp4 expression, but also for normal anterior neural patterning and eye formation.

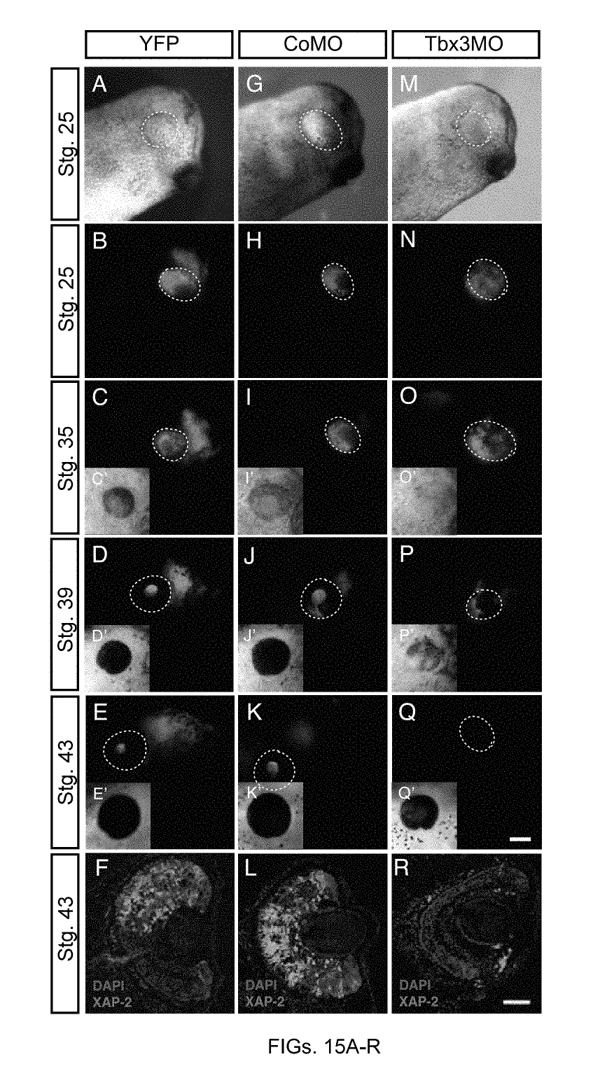

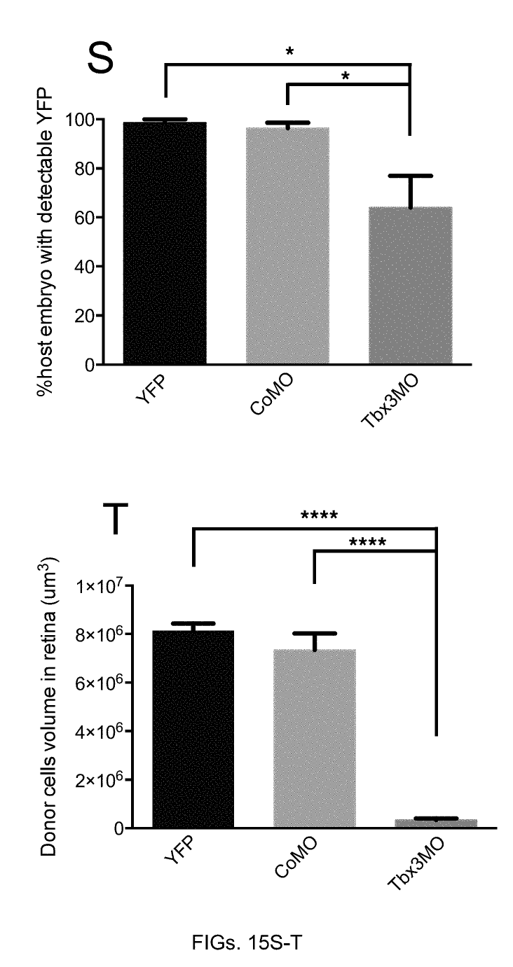

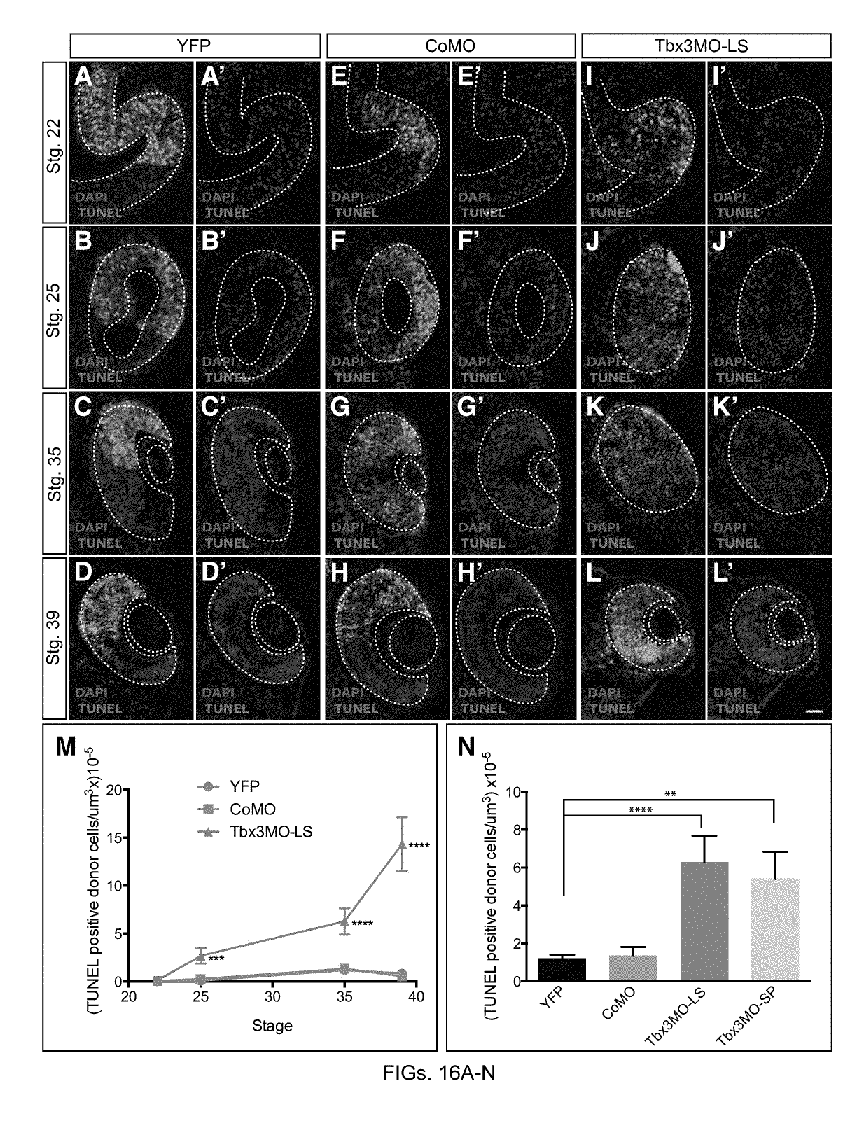

To address the question of why Tbx3 knockdown in eye field cells results in eye defects, YFP alone, and in combination with CoMO or Tbx3MO, were injected into one blastomere of donor embryos at the 8-cell stage, grown to stage 15, and a centrally located portion of the YFP-positive donor eye field was grafted into the eye field of uninjected, host embryos. The fate of YFP-positive donor cells was then monitored by fluorescence in living embryos as they grew into tadpoles (FIGS. 15A-T). YFP-positive donor cells were detected at all developmental stages in tadpoles that received donor eye fields from YFP-only and YFP plus CoMO injected embryos (FIGS. 15A-E, G-K). In contrast, a significant reduction in the number of embryos with detectable YFP was observed by stage 39 in embryos that had received eye fields from Tbx3MO injected embryos (FIGS. 15M-Q, S). At stage 43, tadpoles were sectioned and the volume of the YFP+ donor cells in host retinas indicated a dramatic reduction in the number of YFP positive cells from Tbx3MO injected transplants, versus YFP-only or YFP plus CoMO donor eye fields (FIGS. 15F,L,R and T). No increase in YFP fluorescence was observed outside the eye in either intact or sectioned embryos, suggesting the reduced YFP expression in tadpoles that received YFP/Tbx3MO transplants was not due to simple migration of the cells out of the eye. To determine if cell death might explain the loss of donor eye field cells, embryos receiving transplants were sectioned and TUNEL-staining performed (FIGS. 16A-N). At optic vesicle stage (stg. 22) YFP-positive donor cells were detected, vesicle morphology appeared normal, and no TUNEL-positive cells were detected in transplants derived from untreated, CoMO, or Tbx3MO-LS injected hosts (FIGS. 16A, A′, E, E′, I, I′ and M). From stage 25 to 39 however, there was a significant increase in the number of TUNEL-positive donor eye field cells transplanted from host embryos injected with Tbx3MO-LS (FIG. 16M). In addition, lens and eye formation appeared delayed, and eyes were smaller in embryos receiving YFP/Tbx3MO-LS eye field transplants (Compare FIGS. 16B-D′ and F-H′ to J-L′). A similar number of TUNEL positive cells were detected at stage 35 when the splice-blocking morpholino Tbx3MOSP was used to knockdown Tbx3 expression in eye field cells (FIG. 16N). From these results, it was concluded that knockdown of Tbx3 in eye field cells, ultimately resulted in their death during the late optic vesicle and optic cup stages of eye development.

### Example 9—Neither is Sufficient, but Together Tbx3 and Pax6 Drive Pluripotent Cells to Form Retina

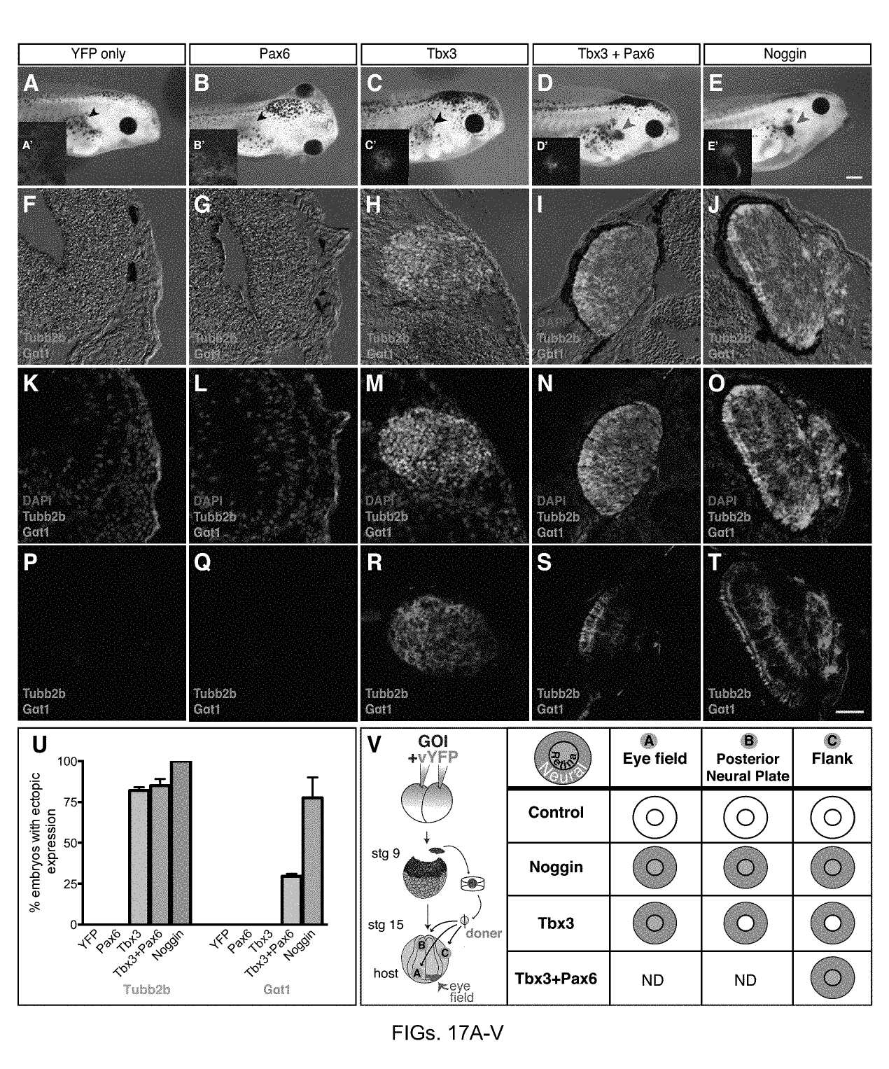

Noggin requires Tbx3 for neural and retinal induction (FIGS. 6A-J′ and 7A-T). However, unlike Noggin, Tbx3 is not sufficient to convert pluripotent cells to a retinal fate outside of the eye field (FIGS. 7A-T and 10A-N), indicating that in addition to repressing BMP4, Noggin must have an additional activity that Tbx3 lacks. It was previously demonstrated that Noggin induces pax6 transcription, while Tbx3 does not (Zuber et al., “Specification of the Vertebrate Eye by a Network of Eye Field Transcription Factors,” Development 130:5155-5167 (2003), which is hereby incorporated by reference in its entirety). Whether Tbx3 and Pax6 could generate retina from pluripotent cells was investigated (FIGS. 17A-V). Similar to YFP alone, Pax6 expressing cells generated skin epidermis in flank transplants (FIGS. 17A, F and B, G, respectively). Neither the neural marker Tubb2b nor the rod photoreceptor marker rod transducin were detected in YFP (n=29) or Pax6 (n=40) expressing cells (FIGS. 17K, P,L, Q and U). Despite the fact Tbx3 could induce the expression of Tubb2b, rod transducin was never detected (FIGS. 17C, H, M, R and U, n=29). In striking contrast, co-expression of Pax6 with Tbx3 not only induced the expression rod transducin, but the cells organized into an eye-like structure (FIGS. 17D, I,N, n=40). Rod transducin expressing cells were detected adjacent to the pigmented RPE (FIGS. 171, N and S), similar to that observed in the ectopic eyes generated from Noggin expressing cells (FIGS. 17E, J, O, T and U, n=40). These results suggest, that in addition to inhibiting BMP signaling, Noggin (but not Tbx3) also induces Pax6 expression, which is sufficient when combined with Tbx3 to drive retinal specification (FIG. 17V).

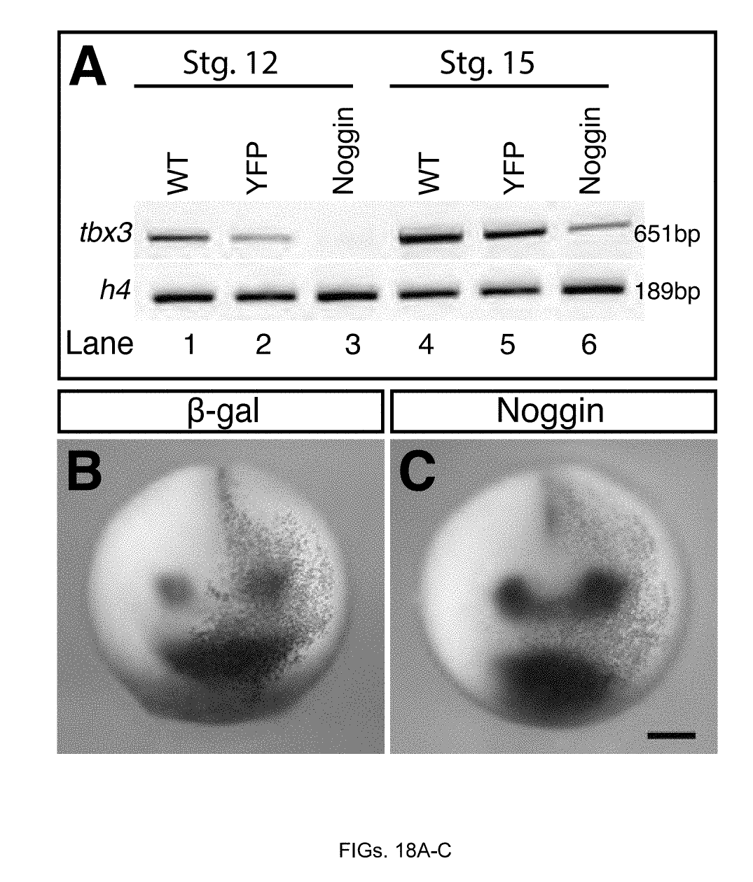

TABLE 1
Primers Sets Used For PCR Analysis.

| | | SEQ | | |
|---|---|---|---|---|
| Primer | | ID | | Target (To |
| Name | Sequence | NO: | Reference | Generate) |
| Tbx3LMO- | 5′-GATCGGATCCAGAAGTTGCTGCTTG-3′ | 8 | This Study | tbx3.L 5′UTR |
| F | | | | |
| Tbx3LMO- | 5′-GATCCCATGGTCACT1TATCTCACAGC- | 9 | This Study | (pCS2R.Tbx3.L- |
| R | 3′ | | | vYFP) |
| Tbx3SMO- | 5′-GATCGGATCCAGATGTTGCTGCTTG-3′ | 10 | This Study | tbx3.S 5′UTR |
| F | | | | |
| Tbx3SMO- | 5′-GATCCCATGGTCACTTGCACCCTTTG-3′ | 11 | This Study | (pCS2R.Tbx3.S- |
| R | | | | vYFP) |
| GR-F | 5′-GGCCATATGCCCTCTGAAAATCTG-3′ | 12 | This Study | hGR of pCS2 + Tbx5- |
| | | | | EnR-GR |
| GR-R | 5′-AGTTCTAGAGGCTCGAGGTTTTTTG-3′ | 13 | (Horb and | (pCS2R.XITbx3GR) |
| | | | Thomsen, | |
| | | | 1999) | |
| Tbx3LDBD- | 5′-GCTCGAATTCAAGGCCGAGCTG-3′ | 14 | This Study | tbx3.L DBD |
| F | | | | |
| Tbx3LDBD- | 5′-GATCCTCGAGCTACTCTCCTCGTCAC-3′ | 15 | This Study | (pCS2R.Tbx3.L.DB |
| R | | | | D-EnR-GR) |
| EnR-GR-F | 5′-GTTTAAAGAATTCATGGCCCTGG-3′ | 16 | This Study | EnR-GR of |
| | | | | pCS2 + Tbx5-EnR- |
| | | | | GR |
| EnR-GR-R | 5′-AGTTCTAGAGGCTCGAGGTTTTTTG-3′ | 17 | This Study | pCS2R.Tbx3.L.DBD- |
| | | | | EnR-GR |
| Tbx3L(3′- | 5′-GGCAGACACTATCAGCCTGCC-3′ | 18 | This Study | tbx3.L 3′UTR |
| UTR)-F | | | (FIG. 2) | |
| Tbx3L(3′- | 5′-GCACAGGCCTATGATAAAGTTATCCC- | 19 | This Study | tbx3.L 3′UTR |
| UTR)-R | 3′ | | | |
| Tbx3S(5′- | 5′-TACAGAACCCGGACTGTCCCAGTCA-3′ | 20 | This Study | tbx3.S 5′UTR |
| UTR)-F | | | (FIG. 2) | |
| Tbx3S(5′- | 5′-CTG1TCCCAGAGATCCTTGGCTTCC-3′ | 21 | This Study | tbx3.S 5′UTR |
| UTR)-R | | | | |
| H4-F | 5′-CGGGATAACATTCAGGGTATCACT-3′ | 22 | (Hollemann | histone H4 |
| | | | et al., 1998) | |
| H4-R | 5′-ATCCATGGCGGTAACTGTCTTCCT-3′ | 23 | (FIGS. 2, 10, | histone H4 |
| | | | 5 & 18) | |
| NCAM-F | 5′-CACAGTTCCACCAAATGC-3′ | 24 | Xenbase | ncam1 |
| | | | (FIG. 10) | |
| NCAM-R | 5′-GGAATCAAGCGGTACAGA-3′ | 25 | Xenbase | ncam1 |
| | | | (FIG. 10) | |
| Tubb2b-F | 5′-ACACGGCATTGATCCTACAG-3′ | 26 | Xenbase | tubb2b |
| | | | (FIG. 10) | |
| Tubb2b-R | 5′-AGCTCCTTCGGTGTAATGAC-3′ | 27 | Xenbase | tubb2b |
| | | | (FIG. 10) | |
| Xbra-F | 5′-GGATCGTTATCACCTCTG-3′ | 28 | Xenbase | t (Xbra) |
| | | | (FIG. 10) | |
| Xbra-R | 5′-GTGTAGTCTGTAGCAGCA-3′ | 29 | Xenbase | t (Xbra) |
| | | | (FIG. 10) | |
| actc1-F | 5′-GCTGACAGAATGCAGAAG-3′ | 30 | Xenbase | actc1 |
| | | | (FIG. 10) | |
| actc1-R | 5′-TTGCTTGGAGGAGTGTGT-3′ | 31 | Xenbase | actc1 |
| | | | (FIG. 10) | |
| Tbx3(Ex1)- | 5′-CCAGTAATTTCAGGGTCAGGC-3′ | 32 | This Study | exon 1 tbx3.L and |
| F | | | (F in FIG. 5; | tbx3.S |
| | | | FIG. 18) | |
| Tbx3(Ex2)- | 5′-AAGAACACTCACAAATCATG-3′ | 33 | This Study | exon 2 tbx3.L and |
| R | | | (FIG. 18) | tbx3.S |
| Tbx3S(intr1)- | 5′-GCTGTTCTGTATTAAAGTCCTGG-3′ | 34 | This Study | intron 1 tbx3.S |
| R2 | | | (R1 in FIG. | |
| | | | 5) | |
| Tbx3L(intr1)- | 5′-GGAAAGGAGATAACACGAGTTGG-3′ | 35 | This Study | intron 1 tbx3.L |
| R1 | | | (R2 in FIG. | |
| | | | 5) | |

Hollemann et al., “The Xenopus homologue of the Drosophila gene tailless has a function in early eye development,” Development 125, 2425-2432 (1998), which is hereby incorporate by reference in its entirety.
Horb, M. E. and Thomsen, G. H. “Tbx5 is essential for heart development.,” Development 126, 1739-1751 (1999), which is hereby incorporate by reference in its entirety.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of producing an enriched preparation of neural progenitor cells from a population of pluripotent stem cells, said method comprising:
administering Tbx3 to the population of pluripotent stem cells and
culturing the population of pluripotent stem cells, to which Tbx3 has been administered, under conditions suitable to produce the enriched preparation of neural progenitor cells from the population of pluripotent stem cells.

2. The method of claim 1, wherein the method is carried out in vitro.

3. The method of claim 1, wherein the pluripotent stem cells are embryonic stem (ES) cells, fetal tissue stem cells, or induced pluripotent stem cells (iPSc).

4. The method of claim 1, wherein said Tbx3 is coupled to an intracellular delivery vehicle.

5. The method of claim 1 further comprising:
contacting the enriched preparation of neural progenitor cells produced during or after said culturing with one or more reagents suitable to induce differentiation and production of retinal progenitor cells, neuronal progenitor cells, or glial progenitor cells from the preparation of neural progenitor cells.

6. The method of claim 5, wherein the one or more reagents comprise Pax6 and said contacting induces differentiation and production of retinal progenitor cells.

7. The method of claim 1 further comprising:
isolating neural progenitor cells from the population of pluripotent stem cells after said culturing.

8. An enriched preparation of neural progenitor cells produced in accordance with the method of claim 1.

9. A method of treating a retinal disorder, said method comprising:
selecting a subject having retinal disorder, and
administering, to said subject, the enriched preparation of neural progenitor cells of claim 8.

10. The method of claim 9, wherein the retinal disorder is a degenerative eye disease selected from the group consisting of age-related macular degeneration, retinitis pigmentosa and cone-rod dystrophies.

11. A method of producing an enriched preparation of retinal progenitor cells from a population of stem cells, said method comprising:
administering Tbx3 and Pax6 to the population of stem cells and
culturing the population of stem cells, to which Tbx3 and Pax6 have been administered, under conditions suitable to produce the enriched preparation of retinal progenitor cells from the population of stem cells.

12. The method of claim 11, wherein the method is carried out in vitro.

13. The method of claim 11, wherein the stem cells are pluripotent stem cells selected from the group consisting of embryonic stem (ES) cells, fetal tissue stem cells, or induced pluripotent stem cells (iPSc).

14. The method of claim 11, wherein said Tbx3 and/or Pax6 are coupled to an intracellular delivery vehicle.

15. The method of claim 14, wherein the intracellular delivery vehicle is selected from the group consisting of a cell penetrating peptide, a cationic amphiphilic-based delivery reagent, and a nanoparticle delivery vehicle.

16. The method of claim 11, wherein said culturing is carried out under conditions suitable for retinal organoid formation.

17. A preparation of retinal organoids formed in accordance with the method of claim 16.

18. The method of claim 11 further comprising:
administering said enriched preparation of retinal progenitor cells formed during said culturing to the eye of a subject in need thereof.

19. An enriched preparation of retinal progenitor cells produced in accordance with the method of claim 11.

20. A method of treating a retinal disorder, said method comprising:
selecting a subject having a retinal disorder, and
administering, to said subject, the enriched preparation of retinal progenitor cells of claim 19.
